(19) United States
(12) Patent Application Publication
(10) Pub. No.: US 2024/0254458 A1
(43) Pub. Date: Aug. 1, 2024

(54) SINGLE-COMPONENT NEAR-INFRARED OPTOGENETIC SYSTEMS FOR GENE TRANSCRIPTION REGULATION

(71) Applicant: Albert Einstein College of Medicine, Bronx, NY (US)

(72) Inventors: Vladislav V. Verkhusha, Bronx, NY (US); Andrii A. Kaberniuk, Bronx, NY (US)

(21) Appl. No.: 18/559,495

(22) PCT Filed: May 18, 2022
(86) PCT No.: PCT/US22/29781

§ 371 Date: Nov. 7, 2023

Related U.S. Application Data
(60) Provisional application No. 63/190,540, filed on May 19, 2021.

(51) Int. Cl. C12N 9/12; C12N 15/113

(52) U.S. Cl. CPC C12N 9/1241; C12N 15/113; C07K 2319/80; C12Y 207/07065

(57) ABSTRACT

This disclosure provides the single-component near-infrared light-controlled IsPadC-PCM-based optogenetic systems for prokaryotic and eukaryotic cells, consisting of an evolved photosensory core module of the Idiomarina sp. bacterial phytochrome, named iLight. The iLight systems are smaller and packable in an adeno-associated virus, as compared to the other near-infrared optogenetic systems based on phytochromes. This disclosure demonstrates the high light-activation efficiency of the developed iLight systems in gene transcription regulation in bacteria, cultured mammalian cells, primary isolated neurons, and living mouse tissue in vivo. The iLight systems also enable crosstalk-free spectral multiplexing with optogenetic systems and fluorescent probes activated or excited by light of the visible spectral range.

### CROSS-REFERENCE TO RELATED APPLICATIONS

This application claims benefit of priority to U.S. Provisional Application No. 63/190,540, filed May 19, 2021. The contents of which are incorporated herein by reference in its entirety.

### STATEMENT REGARDING FEDERALLY SPONSORED RESEARCH

This invention was made with government support under GM122567 awarded by National Institutes of Health. The government has certain rights in the invention.

### FIELD OF THE INVENTION

This disclosure relates generally to systems and methods for light-induced gene transcription control.

### BACKGROUND OF THE INVENTION

Light-induced protein-protein interactions exploited in the non-opsin optogenetic tools is include homodimerization, heterodimerization, and oligomerization. Homodimerization of a small light-oxygen-voltage (LOV)-domain-containing protein, called VVD, is used for light-controlled transcription. A LOV2 domain of phototropin 1 from Avena sativa and a modified PDZ domain have been combined into an optogenetic system based on heterodimerization. LOV2-based optogenetic tools enable light control of nuclear-cytoplasmic protein shuttling. Cryptochrome 2 (CRY2) from Arabidopsis thaliana is another photoreceptor, which initially was applied with CIB1 partner in two-component heterodimerization approaches. Later, its natural oligomerization ability was used in optogenetic clustering approaches. Further tuning of the engineered light-activatable systems led to a design of the new generation of photodimerizers for advanced control of protein localization, cell signaling, and recombinase activity. All these optogenetic systems sense 440-480 nm light. Therefore, systems sensing light in a different spectral range are required for simultaneous use with blue-light-controlled optogenetic tools.

A class of photoreceptors called phytochromes stands apart from other photosensing proteins because of their ability to absorb far-red or near-infrared (NIR) light. All phytochromes utilize heme-derived linear tetrapyrrole compounds as their light-sensing chromophores. Red-light-triggered heterodimerization of a plant phytochrome B (PhyB) and a phytochrome-interacting factor 6 (PIF6) from Arabidopsis has been successfully applied to transcriptional control, cell signaling, and protein localization. Unlike plant phytochromes, which use phytochromobilin or phycocyanobilin tetrapyrroles as a chromophore, a subclass of bacterial phytochrome photoreceptors (BphPs) incorporate biliverdin IXa (BV) tetrapyrrole. As BV has the largest electron-conjugated system, it absorbs the most NIR-shifted light among all chromophores found in phytochromes. Moreover, in contrast to phytochromobilin or phycocyanobilin tetrapyrroles, BV is naturally present in all mammalian cells, which makes BphPs the favorable templates to develop fluorescent proteins for applications in mammals. BphPs exist in two interconvertible states, Pr (absorbs at 660-700 nm) and Pfr (absorbs at 740-780 nm). Upon NIR illumination, BphP-bound BV isomerizes via the fourth D-ring rotation around its 15-16 double bond. This Z-E isomerization results in the subsequent structural changes in an N-terminal photosensory core module (PCM) and an output (effector) domain of BphP. In turn, the PCM is formed by three domains, PAS (Per-ARNT-Sim), GAF (cGMP phosphodiesterase/adenylate cyclase/FhlA transcriptional activator), and PHY (phytochrome-specific), connected with α-helix linkers.

Recently, the first optogenetic system that uses BphP from Rhodopseudomonas palustris, called RpBphP1, was developed. The NIR light-triggered heterodimerization of the full-length RpBphP1 with its natural RpPpsR2 or engineered QPAS1 binding partners allows precise control of gene transcription. BphP, serving as a light-sensing element of the RpBphP1-RpPpsR2 optogenetic system, belongs to non-canonical (bathy) BphPs, which in darkness adopt the Pfr state. Under NIR light of 740-780 nm, it undergoes the Pfr→Pr photoconversion, resulting in the reversible binding of RpPpsR2.

The significant drawback of the currently available NIR optogenetic systems is the requirement to co-express two large protein components (i.e., PhyB phytochrome and PIF6 partner or RpBphP1 phytochrome and RpPpsR2 partner), which require co-transfection with two plasmids or co-transduction with two adeno-associated viruses (AAVs) (Redchuk, T. A., et al. Nat Protoc 13, 1121-1136 (2018)). Another substantial drawback is a rather high background in darkness, for example, in the RpBphP1-RpPpsR2 system.

Therefore, there is a strong need for a novel strategy for light-induced gene transcription control.

### SUMMARY OF THE INVENTION

This disclosure addresses the need mentioned above in a number of aspects. In one aspect, this disclosure provides a polynucleotide, comprising a nucleotide sequence encoding a chimeric polypeptide comprising a light-responsive polypeptide linked to a DNA binding domain, wherein the light-responsive polypeptide comprises an amino acid sequence having at least 90% sequence identity to SEQ ID NO: 1 or comprises the amino acid sequence of SEQ ID NO: 1.

In some embodiments, the light-responsive polypeptide is a variant of Idiomarina sp. A28L Phytochrome activated diguanylyl Cyclase (IsPadC). In some embodiments, the light-responsive polypeptide comprises an N-terminal photosensory core module (PCM) of IsPadC.

In some embodiments, the light-responsive polypeptide comprises at least one mutation at position I68, H80, A86, R90, S242, R274, R295, 1360, or L464. In some embodiments, the at least one mutation comprises one or more substitutions selected from the group consisting of I68F, H80Q, A86T, R90S, S242C, R274K, R295H, I360V, L464V, and combinations thereof. In some embodiments, the at least one mutation comprises at least one of I68F, R295H, and L464V substitutions. In some embodiments, the at least one mutation comprises the F68I, H295R, and V464L substitutions.

In some embodiments, the light-responsive polypeptide comprises an amino acid sequence having at least 90% sequence identity to SEQ ID NO: 2 or comprises the amino acid sequence of SEQ ID NO: 2.

In some embodiments, the light-responsive polypeptide is linked to the DNA binding domain via a peptide linker. In some embodiments, the nucleotide sequence is operably linked to a promoter.

In some embodiments, the DNA binding domain comprises a DNA binding motif. In some embodiments, the DNA binding motif comprises a helix-turn-helix, a homeodomain, a leucine zipper, a helix-loop-helix, or a zinc finger. In some embodiments, the DNA binding domain comprises a Gal4 DNA binding domain, a Lex-A DNA binding domain, an NF-κB DNA binding domain, a cro repressor DNA binding domain, a lac repressor DNA binding domain, a GCN4 DNA binding domain, an Opaque-2 DNA binding domain, or a TGAIa DNA binding domain.

In some embodiments, the light-responsive polypeptide when associated with a chromophore is capable of switching from a first state to a second state when exposed to illumination by a first wavelength and switching from the second state to the first state when exposed to illumination by a second wavelength or returning from the second state to the first state in darkness. In some embodiments, the chromophore is a biliverdin chromophore.

In some embodiments, the first state is a Pr state and the second state is a Pfr state. In some embodiments, at least a portion of the light-responsive polypeptide exists in a dimeric form in the first state. In some embodiments, at least a portion of the light-responsive polypeptide exists in a tetrameric form in the second state.

In some embodiments, at least a pair of the DNA binding domains of the tetrameric form of the light-responsive polypeptide are capable of binding to a DNA recognition site.

In some embodiments, a PHY-tongue of only one protomer of the dimeric form of the light-responsive polypeptide that is constituted by two anti-parallel β-sheets in the first state is restructured to an α-helix in the second state when exposed to illumination by the first wavelength.

In some embodiments, the first wavelength and the second wavelength are in far-red and near-infrared spectrum. In some embodiments, the first wavelength is between about 600 nm and about 680 nm (e.g., 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm). In some embodiments, the first wavelength is about 660 nm. In some embodiments, the second wavelength is between about 740 nm and about 800 nm (e.g., 740 nm, 745 nm, 750 nm, 755 nm, 760 nm, 765 nm, 770 nm, 775 nm, 780 nm, 785 nm, 790 nm, 795 nm, 800 nm). In some embodiments, the second wavelength is about 780 nm.

In some embodiments, the polynucleotide further comprises a second nucleotide sequence encoding a second light-responsive polypeptide. In some embodiments, the second light-responsive polypeptide comprises rhodopsin.

In another aspect, this disclosure also provides a light-responsive polypeptide comprising an amino acid sequence having at least 90% sequence identity to SEQ ID NO: 2, 3 or 4 or comprising the amino acid sequence of SEQ ID NO: 2, 3 or 4.

In some embodiments, the light-responsive polypeptide is a variant of a PCM of an IsPadC. In some embodiments, the light-responsive polypeptide comprises at least one mutation at position I68, H80, A86, R90, S242, R274, R295, 1360, or L464. In some embodiments, the at least one mutation comprises one or more substitutions selected from the group consisting of I68F, H80Q, A86T, R90S, S242C, R274K, R295H, I360V, L464V, and combinations thereof. In some embodiments, the at least one mutation comprises at least one of F68I, H295R, V464L substitutions. In some embodiments, the at least one mutation comprises the F68I, H295R, and V464L substitutions.

In some embodiments, the light-responsive polypeptide further comprises a DNA binding domain linked to the amino acid sequence. In some embodiments, the light-responsive polypeptide further comprises a DNA binding domain linked to the amino acid sequence via a linker. In some embodiments, the DNA binding domain comprises a DNA binding motif. In some embodiments, the DNA binding motif comprises a helix-turn-helix, a homeodomain, a leucine zipper, a helix-loop-helix, or a zinc finger. In some embodiments, the DNA binding domain comprises a Gal4 DNA binding domain, a Lex-A DNA binding domain, an NF-κB DNA binding domain, a cro repressor DNA binding domain, a lac repressor DNA binding domain, a GCN4 DNA binding domain, an Opaque-2 DNA binding domain, or a TGAIa DNA binding domain.

In some embodiments, the light-responsive polypeptide is associated with a chromophore and capable of switching from a first state to a second state when exposed to illumination by a first wavelength and switching from the second state to the first state when exposed to illumination by a second wavelength, or returning from the second state to the first state in darkness. In some embodiments, the chromophore is a biliverdin chromophore.

In some embodiments, the first state is a Pr state and the second state is a Pfr state. In some embodiments, at least a portion of the light-responsive polypeptide exists in a dimeric form in the first state. In some embodiments, at least a portion of the light-responsive polypeptide exists in a tetrameric form in the second state. In some embodiments, at least a pair of the DNA binding domains of the tetrameric form of the light-responsive polypeptide are capable of binding to a DNA recognition site. In some embodiments, the PHY-tongue of only one protomer of the dimeric form of the light-responsive polypeptide that is constituted by two anti-parallel β-sheets in the first state is restructured to an α-helix in the second state when exposed to illumination by the first wavelength.

In some embodiments, the first wavelength and the second wavelength are in far-red or near-infrared spectrum. In some embodiments, the first wavelength is between about 600 and about 680 nm. In some embodiments, the first wavelength is about 660 nm. In some embodiments, the second wavelength is between about 740 and about 800 nm. In some embodiments, the second wavelength is about 780 nm.

Also provided in this disclosure are (i) a vector comprising a polynucleotide described above; (ii) a host cell comprising a polynucleotide or a vector, as described above; (iii) a polypeptide encoded by a polynucleotide described above; and (iv) a composition comprising a polynucleotide, a vector, a host cell, or a polypeptide, as described above.

In another aspect, this disclosure further provides a system for modulating an expression level of a gene. The system comprises a polynucleotide, a vector, a host cell, or a polypeptide, as described above, wherein the DNA binding domain is capable of binding to a regulatory element of the gene. In some embodiments, the regulatory element is a promoter or an operator.

In another aspect, this disclosure additionally provides a method for modulating a gene expression level. The method comprises: (a) introducing a polynucleotide or a vector, as described above, to a cell; and (b) exposing the cell to illumination by a first wavelength and optionally exposing the cell to illumination by a second wavelength, wherein the DNA binding domain is capable of binding to a regulatory element of the gene. In some embodiments, the regulatory element is a promoter or an operator.

Also provided in this disclosure is a method for modulating a gene expression level, comprising: (a) providing a polypeptide or a host cell, as described above; and (b) exposing the polypeptide or the host cell to illumination by a first wavelength and optionally exposing the cell to illumination by a second wavelength, wherein the DNA binding domain is capable of binding to a regulatory element of the gene.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, and 1D are a set of diagrams showing molecular evolution of the IsPadC-PCM variants in bacteria. FIG. 1A shows the schematics outlining the high-throughput screening of IsPadC-PCM mutants with the light-controlled behavior. FIG. 1B shows the plasmids used in the molecular evolution of IsPadC-PCM. Left: pLEVI(408)-ColE-IsPadC-PCM encodes light-sensing protein LexA408-DBD-IsPadC-PCM-msfGFP under constitutive J23116 promoter and mCherry reporter under constitutive ColE promoter controlled by LexA408 operator. Right: pWA23h-bla encodes heme oxygenase for biliverdin production under constitutive β-lactamase promoter. FIG. 1C shows repression of the mCherry reporter expression with 660 nm light. Streaks of bacteria expressing the indicated IsPadC-PCM mutants were grown on Petri dishes either in darkness or under 660 nm light and then imaged using 570/30 nm excitation and 615/40 nm emission filters. FIG. 1D shows efficiency of the mCherry repression by selected IsPadC-PCM variants. mCherry signal was measured in bacterial suspensions grown in darkness or under 660 nm light. Box plots show the median (center line), first and third quartiles (box edges), 1× the standard deviation (whiskers), and individual data points. n=4 independent experiments. a.u., arbitrary units.

FIGS. 2A, 2B, 2C, 2D, and 2E are a set of diagrams showing an iLight-based system for the light-induced repression of protein expression in bacteria. FIGS. 2A and 2B show dependence of the inhibition efficiency on the duration of Off (FIG. 2A) and On (FIG. 2B) time of the 660 nm illumination. FIG. 2C shows the effect of 780 nm light, which photoconverts iLight from the Pfr state to the Pr state, on the inhibition of the mCherry reporter production. Box plots show the median (center line), first and third quartiles (box edges), 1× the standard deviation (whiskers), and individual data points. The illumination steps described in (a-c) were repeated in the loop, and the total duration of each of these experiments was 16 h. FIG. 2D shows inhibition of the mCherry reporter expression in the ongoing mCherry expression conditions. Bacterial samples expressing the mCherry reporter were cultured in darkness and then transferred to 660 nm light for the indicated illumination time periods, after which the fluorescence intensity of the bacteria was analyzed. Each bacterial sample was cultured for totally 24 h before the analysis. Error bars, s.d. (n=4 independent experiments). FIG. 2E shows efficiency of the inhibition of the mCherry reporter production by various single-point mutants of iLight. Error bars, s.d. (n=4 independent experiments. a.u., arbitrary units.

FIGS. 3A, 3B, 3C, 3D, 3E, and 3F are a set of diagrams showing spectral and photochemical properties of the purified iLight protein. FIG. 3A shows absorbance spectra of iLight in the ground Pr state (solid line) and after the photoconversion to the Pfr state (dashed line). FIG. 3B shows action spectrum of the Pr→Pfr photoconversion upon irradiation with light of specific wavelength measured as the relative decrease of the Pr state absorption at 704 nm. FIG. 3C shows dependence of the half-time of the Pr→Pfr photoconversion on the intensity of 660/15 nm light. FIG. 3D shows dependence of the half-time of the Pfr→Pr photoconversion on the intensity of 780/30-nm light. FIG. 3E shows absorbance of iLight in the Pr state during repeated illumination cycles with 660/15 nm light and then with 780/30 nm light. Absorbance was measured at 704 nm. FIG. 3F shows a native PAGE of wild-type IsPadC-PCM and iLight. Top: Proteins were illuminated with either 780 nm (photoconverting to the Pr state) or 660 nm light (photoconverting to the Pfr state) for 30 min and then ran at 20 μg of the protein per lane in darkness. Bottom: ZnCl₂ staining of the same gel visualizes the amount of the biliverdin chromophore covalently bound to each oligomeric fraction of the proteins. Arrows indicate the bands of the respective oligomeric states of the proteins. Each experiment was independently repeated three times with similar results. a.u., arbitrary units (FIGS. 3A, 3B, and 3E).

FIGS. 4A and 4B are a set of diagrams showing a proposed mechanism of action of the iLight optogenetic systems. FIG. 4A shows the schematics of the mCherry gene transcription repression in the iLight bacterial system. Expression of mCherry from the constitutively active ColE promoter with LexA408 operator is controlled by the oligomeric state of iLight fused with DNA-binding domain of LexA408. FIG. 4B shows the schematics of the reporter gene transcription activation in the iLight mammalian system. To induce reporter expression from the plasmid with 12× (upstream activating sequence) UAS upstream of a minimal promoter, a nucleus localization signal (NLS)-tagged iLight was fused with a Gal4 DNA-binding domain and a VP16 transcriptional activation domain. The 660 nm light-induced iLight tetramerization brings the Gal4 DNA-binding domains into proximity, enabling them to bind 12×UAS and allowing VP16 to recruit transcription initiation complexes.

FIGS. 5A, 5B, and 5C are a set of diagrams showing iLight-induced gene transcription activation in mammalian cells. FIG. 5A shows dependence of the SEAP reporter expression on concentration of the exogenous biliverdin in HeLa cells expressing the iLight optogenetic system. SEAP signal was detected after 48 h of 660 nm illumination. FIG. 5B shows the 72 h long kinetics of the SEAP reporter expression in HeLa cells with the iLight optogenetic system. Cells were illuminated in one of the following regimes: cells were kept in darkness; illumination with 660 nm is light for 72 h; illumination with 660 nm light for 24 h followed by 48 h of darkness; or illumination with 660 nm light for 24 h followed by 4 h of 780 nm light and 44 h of darkness. The culture medium samples were collected every 12 h to measure the SEAP signal. FIG. 5C shows dependence of the SEAP reporter expression on the power of 660 nm activation light. HeLa cells were kept in darkness or under 660 nm light of the respective light intensities. Numbers indicate fold increase in the SEAP signal over darkness. In FIGS. 5B and 5C, cells were supplemented with 10 μM biliverdin. In FIGS. 5A, 5B, and 5C, HeLa cells bearing NLS-Gal4-DBD-iLight-VP16 were transfected with pG12-SEAP (12×UAS) reporter plasmid. Box plots show the median (center line), first and third quartiles (box edges), 1× the standard deviation (whiskers), and individual data points. n=4 independent experiments for all conditions. a.u., arbitrary units.

FIGS. 6A, 6B, 6C, 6D, 6E, and 6F are a set of diagrams showing iLight-induced gene transcription activation in primary cultured neurons. Murine hippocampal neurons were co-transduced with the iLight optogenetic system and the mCherry and CheRiff reporter AAVs. FIGS. 6A and 6B show representative images of the neurons transduced with iLight system and mCherry AAVs (top images) or with mCherry reporter AAV only (bottom images) incubated for 5 days either under 660 nm light (FIG. 6A) or in darkness (FIG. 6B). Scale bar, 20 m. Experiments (FIGS. 6A and 6B) were independently repeated three times with similar results. FIG. 6C shows averaged mCherry reporter fluorescence in neurons cultured under 660 nm light (n=53 cells) or in darkness (n=62 cells) after subtraction of average fluorescence in cells transduced with mCherry AAV alone. The difference between groups was significant (paired two-sided Student's t-test, exact P values: T=6, df=113, P=2.3×10⁻⁸). The data from a typical experiment are presented. Error bars, SEM; a.u., arbitrary units. FIGS. 6D and 6E show representative photocurrent traces in neurons co-transduced with iLight system and CheRiff reporter AAVs incubated either under 660 nm light (FIG. 6D) or in darkness (FIG. 6E) and exposed to 0.5 s of 505 nm light of 200 mW cm⁻² during recording. The cell responds with a small photocurrent because the iLight system is inactive in darkness (FIG. 6E). Neurons in FIGS. 6D and 6E were voltage-clamped at −70 mV, and the photocurrent traces were smoothed by moving the average filter with 2 ms window. FIG. 6F shows the effect of iLight system on photocurrent densities (current normalized by membrane capacitance) in the neurons expressing CheRiff and incubated either under 660 nm light or in darkness (n=10 cells in each group). The difference between the two groups of neurons was significant (paired two-sided Student's t-test, exact P values: T=2.17, df+18, P=0.044). Average photocurrents in the cells expressing CheRiff alone, without the iLight system, were subtracted before statistical analysis. Error bars, SEM. Culture medium contained 2 μM biliverdin.

FIGS. 7A and 7B are a set of diagrams showing iLight-induced gene transcription activation in mouse tissue in vivo. FIG. 7A shows RLuc8 luciferase reporter signals detected in mice after the hydrodynamic co-transfection of the livers with the NLS-Gal4-DBD-iLight-VP16 and pG12-RLuc8 plasmids. Mice kept in darkness (top) or illuminated with 660 nm light of 3.2 mW cm⁻² (bottom) for 48 h are shown. FIG. 7B shows kinetics of the RLuc8 reporter expression in mice shown in FIG. 7A kept in darkness or illuminated for up to 96 h. Box plots show the median (center line), first and third quartiles (box edges), 1× the standard deviation (whiskers), and individual data points. n=3 individual animals.

FIG. 8 shows the results of the screening of FACS selected clones using replica approach. Replicated dishes were grown in the darkness or under 660 nm light. After overnight incubation, dishes were imaged in mCherry and msfGFP and analyzed using ImageJ to find clones with the maximum difference of signal in mCherry channel and the minimum difference in msfGFP channel. White arrows indicate clone 1.3 selected after the first round of mutagenesis with ˜2-fold contrast of dark-to-light mCherry signal.

FIG. 9 shows a pipeline for selection of clones with light-activated repression of mCherry reporter expression. After random mutagenesis of IsPadC-PCM, library of mutated variants were grown in E. coli bacteria in darkness with following enrichment of the double positive clones (gate P3 mCherry and msfGFP positive cells). The enriched library was overnight cultivated under 660 nm light, and mCherry negative, msfGFP positive cells were selected (gate P4) for the following screening.

FIG. 10 shows the initial characterization of the selected clones. Selected after screening clones (clones were selected from both dishes cultivated in the darkness and under 660 nm light) were streaked on dishes and cultivated overnight in the darkness or under 660 nm light. Next, intensity of mCherry signal was analyzed with ImageJ in the selected ROI's with wild-type IsPadC-PCM used as a reference.

FIGS. 11A and 11B are a set of diagrams showing a programmable multichannel 660 nm LED array. Principal scheme (FIG. 11A) and assembled prototype (FIG. 11B) of programmable 6-channel 660 nm LED array used for illumination of bacterial cells.

FIG. 12 shows a comparison of mCherry reporter levels in E. coli bacteria with or without iLight in darkness. The bacteria expressed either the full-length pLEVI(408)-ColE-iLight-msfGFP plasmid (left bar) or the same plasmid with the deleted iLight-msfGFP fragment (middle bar). The mCherry fluorescence intensity was the same in both types of bacteria, indicating that iLight did not decrease the expression level of the mCherry reporter. Control empty bacteria exhibited almost (no fluorescence (right bar). Error bars, s.d. (n=3 independent experiments, a.u., arbitrary units).

FIG. 13 shows dark relaxation of wild-type IsPadC-PCM and iLight. Wild-type IsPadC-PCM and iLight were photoconverted to the Pfr state with 660 nm light, and dynamics of the dark relaxation were further monitored by detecting absorbance at 704 nm. The complete relaxation of iLight was observed after overnight incubation in darkness. Absorbance is shown in arbitrary units, a.u.

FIGS. 14A and 14B are a set of diagrams showing a full native PAGE of IsPadC-PCM and iLight. FIG. 14A shows that proteins were illuminated with either 780 nm (photoconverting to the Pr state) or 660 nm light (photoconverting to the Pfr state) for 30 min and then run at 20 μg of the protein per lane in darkness. FIG. 14B shows ZnCl₂ staining of the same gel visualizes the amount of the biliverdin chromophore covalently bound to each oligomeric fraction of the proteins. See also FIG. 3F. Experiments were independently repeated three times with similar results.

FIGS. 15A, 15B, 15C, 15D, 15E, and 15F (collectively “FIG. 15”) are a set of diagrams showing the results of size-exclusion chromatography of the purified IsPadC-PCM and iLight proteins. IsPadC-PCM (FIGS. 15A and 15B) and iLight (FIGS. 15D and 15E) purified from bacteria were illuminated with either 780 nm light (photoconverting it to the Pr state) (FIGS. 15A and 15D) or 660 nm light (photoconverting it to the Pfr state) (FIGS. 15B and 15E) for 30 min at r.t. and then applied to size-exclusion chromatography at 1.9 mg/ml in darkness. The major elution peaks in FIGS. 15A, 15B, 15D, and 15E correspond to the protein dimers. After activation with 660 nm light ˜25% of iLight protein elutes as a tetramer (arrow in FIG. 15E). FIGS. 15C and 15F show molecular weight (MW) markers aligned with the elution profiles of IsPadC-PCM (FIG. 15C) and iLight (FIG. 15F).

FIG. 16 shows example data from a single experiment using murine hippocampal neurons co-transduced with iLight optogenetic system and mCherry reporter AAVs. mCherry reporter fluorescence intensity (in arbitrary units, a.u) in individual neurons cultured under 660 nm light or in darkness, with or without iLight is indicated as dots. Biliverdin concentration was 2 μM.

FIG. 17 shows murine hippocampal neurons were transduced with AAV encoding near-infrared fluorescent protein miRFP680 at DIV7 and imaged on DIV14 (617 nm LED, 620/15 nm excitation filter, 660LP dichroic mirror, 700/50 nm emission filter). The mean fluorescence value was 5303 arbitrary units (a.u.), standard deviation 2205 a.u., n=112 cells, coefficient of variation 42%. No biliverdin was added. Error bars indicate SEM.

FIGS. 18A, 18B, 18C, and 18D (collectively “FIG. 18”) are a set of diagrams showing effects of near-infrared and green light on photocurrents and voltage in neurons co-expressing CheRiff and iLight. FIG. 18A shows voltage trace during exposure to 656 nm LED light. FIG. 18B shows voltage trace during exposure to 505 nm light. FIG. 18C shows current trace during exposure to 656 nm light (the neuron was held at −70 mV in voltage clamp mode). FIG. 18D shows current trace during exposure to 505 nm light (the neuron was held at −70 mV in voltage clamp mode).

FIG. 19 shows examples of action potentials fired by neurons in response to current injection. Murine hippocampal neurons transduced with iLight AAV were patch clamped at DIV14 and held in current clamp mode (zero current). Current (125 pA for cell #1 and 75 pA for cell #2) was injected through the electrode to induce action potentials.

FIGS. 20A and 20B are a set of diagrams showing the three most critical for the iLight functioning mutation mapped on the structure of the activated IsPadC-PCM dimer (PDB ID: 6ET7). Side (FIG. 20A) and top (FIG. 20B) views are shown. The three critical amino acid residues Ile68, Arg295, and Leu464, which are mutated in iLight, are shown in purple with the side chains. The biliverdin chromophore is in cyan. The activated protomer in the Pfr state is in green, and the non-activated protomer, which has remained in the Pr state after the 660 nm illumination, is in red. The N-termini of both protomers are highlighted in the lighter hues.

### DETAILED DESCRIPTION OF THE INVENTION

Near-infrared (NIR) optogenetic systems for transcription regulation are in high demand because NIR light exhibits low phototoxicity and low scattering and allows combining with probes of visible range. However, existing NIR optogenetic systems consist of several protein components of large size and multidomain structure, resulting in low efficiency and high background. This disclosure provides single-component NIR light-controlled IsPadC-PCM-based optogenetic systems consisting of an evolved photosensory core module of Idiomarina sp. bacterial phytochrome, named iLight, which are smaller and packable in an adeno-associated virus (AAV). The IsPadC-PCM-based optogenetic system, as disclosed, shows high efficiency in gene transcription regulation in cultured mammalian cells, primary isolated neurons, and intact mouse tissue in vivo. The disclosed optogenetic system is also suitable for crosstalk-free spectral multiplexing with other optogenetic systems, such as channelrhodopsin.

### A. POLYNUCLEOTIDES ENCODING LIGHT-RESPONSIVE POLYPEPTIDES

In one aspect, this disclosure provides a polynucleotide, comprising a nucleotide sequence encoding a chimeric polypeptide comprising a light-responsive polypeptide linked to a DNA binding domain, wherein the light-responsive polypeptide comprises an amino acid sequence having at least 80% (e.g., 80%, 85%, 90%, 91%, 92%, 93%, 94%, 95%, 96%, 97%, 98%, 99%) sequence identity to SEQ ID NO: 1 or 2 or comprises an amino acid sequence of SEQ ID NO: 1 or 2 (see Table 1).

In some embodiments, the light-responsive polypeptide is a variant or fragment of Idiomarina sp. A28L Phytochrome activated diguanylyl Cyclase (IsPadC). In some embodiments, the light-responsive polypeptide comprises an N-terminal photosensory core module (PCM) of sPadC.

The terms “light-responsive” and “light-activated” are used herein interchangeably. The terms “light-responsive polypeptide,” “light-responsive protein,” “light-activated protein,” and “light-activated protein” mean a polypeptide or protein that undergoes a conformational change when exposed to light of an activating wavelength.

TABLE 1
Representative sequences.

| SEQ | | |
|---|---|---|
| ID | | OTHER |
| NO | PROTEIN SEQUENCES | INFORMATION |
| 1 | MAADLGSDDISKLIAACDQEPIHIPNAIQPFGAMLIVE | IsPadC-PCM (wild- |
| | KDTQQIVYASANSAEYFSVADNTIHELSDIKQANINS | type iLight) |
| | LLPEHLISGLASAIRENEPIWVETDRLSFLGWRHENY | |
| | YIIEVERYHVQTSNWFEIQFQRAFQKLRNCKTHNDLI | |
| | NTLTRLIQEISGYDRVMIYQFDPEWNGRVIAESVRQL | |
| | FTSMLNHHFPASDIPAQARAMYSINPIRIIPDVNAEPQ | |
| | PLHMIHKPQNTEAVNLSSGVLRAVSPLHMQYLRNFG | |
| | VSASTSIGIFNEDRLWGIVACHHTKPRAIGRRIRRLLV | |
| | RTVEFAAERLWLIHSRNVERYMVTVQAAREQLSTTA | |
| | DDKHQAHEIVIEHAADWCKLFRCDGIGYLRGEELTT | |
| | YGETPDQTTINKLVEWLEENGKKSLFWHSHMLKED | |
| | APGLLPDGSRFAGLLAIPLKSDADLFSYLLLFRVAQN | |
| | EVRTWAGKPEKLSVETSTGTMLGPRKSFEAWQDEV | |
| | SGKSQPWRTAQLYAARDIARDLLIVADSMQLNLLND | |
| | QLADANENLEKLASFDDLT | |
| 2 | MAADLGSDDISKLIAACDQEPIHIPNAIQPFGAMLIVE | iLight |
| | KDTQQIVYASANSAEYFSVADNTIHELSDFKQANINS | (IsPadC-PCM with |
| | LLPEQLISGLTSAISENEPIWVETDRLSFLGWRHENY | substitutions I68F, |
| | YIIEVERYHVQTSNWFEIQFQRAFQKLRNCKTHNDLI | H80Q, A86T, R90S, |
| | NTLTRLIQEISGYDRVMIYQFDPEWNGRVIAESVRQL | S242C, R274K, |
| | FTSMLNHHFPASDIPAQARAMYSINPIRIIPDVNAEPQ | R295H, I360V, and |
| | PLHMIHKPQNTEAVNLSCGVLRAVSPLHMQYLRNFG | L464V (in bold) |
| | VSASTSIGIFNEDKLWGIVACHHTKPRAIGRRIRHLLV | relative to wild-type |
| | RTVEFAAERLWLIHSRNVERYMVTVQAAREQLSTTA | IsPadC-PCM) |
| | DDKHQAHEIVIEHAADWCKLFRCDGVGYLRGEELT | |
| | TYGETPDQTTINKLVEWLEENGKKSLFWHSHMLKE | |
| | DAPGLLPDGSRFAGLLAIPLKSDADLFSYLLLFRVAQ | |
| | NEVRTWAGKPEKLSVETSTGTMVGPRKSFEAWQDE | |
| | VSGKSQPWRTAQLYAARDIARDLLIVADSMQLNLLN | |
| | DQLADANENLEKLASFDDLT | |
| 3 | 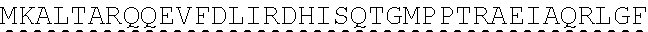 | LexA408:1-87--iLight--- |
| | 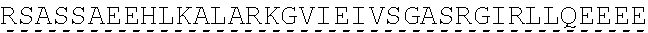 | msfGFP |
| | 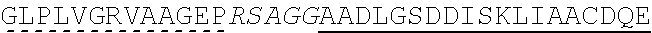 | 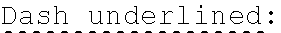 |
| | PIHIPNAIQPFGAMLIVEKDTQQIVYASANSAEYESVA | LexA408:1-87 |
| | DNTIHELSDFKQANINSLLPEQLISGLTSAISENEPIW | Underlined: iLight |
| | VETDRLSFLGWRHENYYIIEVERYHVQTSNWFEIQF | (with substitutions |
| | QRAFQKLRNCKTHNDLINTLTRLIQEISGYDRVMIYQ | I68F, H80Q, A86T, |
| | FDPEWNGRVIAESVRQLFTSMLNHHFPASDIPAQAR | R90S, S242C, R274K, |
| | AMYSINPIRIIPDVNAEPQPLHMIHKPONTEAVNLSC | R295H, I360V, and |
| | GVLRAVSPLHMQYLRNFGVSASTSIGIFNEDKLWGI | L464V relative to wild- |
| | VACHHTKPRAIGRRIRHLLVRTVEFAAERLWLIHSRN | type IsPadC in bold) |
| | VERYMVTVQAAREQLSTTADDKHOAHEIVIEHAAD | Double underlined: |
| | WCKLFRCDGVGYLRGEELTTYGETPDQTTINKLVE | msfGFP |
| | WLEENGKKSLFWHSHMLKEDAPGLLPDGSRFAGLL | Italic: spacing |
| | AIPLKSDADLFSYLLLFRVAQNEVRTWAGKPEKLSVE | residues/linker |
| | TSTGTMVGPRKSFEAWQDEVSGKSQPWRTAQLYAA | |
| | RDIARDLLIVADSMOLNLLNDQLADANENLEKLASF | |
| | DDLTVDSGGGSGGGMVSKGEELFTGVVPILVELDGD | |
| | VNGHKFSVRGEGEGDATNGKLTLKFICTTGKLPVPW | |
| | PTLVTTLTYGVOCESRYPDHMKRHDFFKSAMPEGYV | |
| | QERTISFKDDGTYKTRAEVKFEGDTLVNRIELKGIDF | |
| | KEDGNILGHKLEYNFNSHNVYITADKQKNGIKANFK | |
| | IRHNVEDGSVQLADHYQQNTPIGDGPVLLPDNHYLS | |
| | TQSKLSKDPNEKRDHMVLLEFVTAAGITLGMDELYK | |
| 4 | 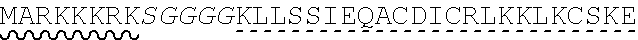 | NLS-Gal4:1-65-- |
| | 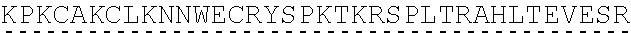 | iLight--VP16--T2A-- |
| | 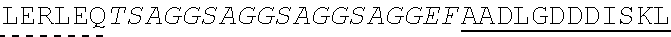 | mTagBFP2 |
| | IAACDQEPIHIPNAIQPFGAMLIVEKDTQQIVYASANS | 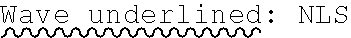 |
| | AEYFSVADNTIHELSDFKQANINSLLPEQLISGLTSAI | 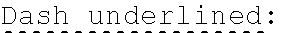 |
| | SENEPIWVETDRLSFLGWRHENYYIIEVERYHVQTSN | Gal4:1-65 |
| | WFEIQFORAFQKLRNCKTHNDLINTLTRLIQEISGYD | Underlined: iLight |
| | RVMIYQFDPEWNGRVIAESVRQLFTSMLNHHFPASDI | (with substitutions |
| | PAQARAMYSINPIRIIPDVNAEPQPLHMIHKPONTEA | I68F, H80Q, A86T, |
| | VNLSCGVLRAVSPLHMQYLRNFGVSASTSIGIFNED | R90S, S242C, R274K, |
| | KLWGIVACHHTKPRAIGRRIRHLLVRTVEFAAERLW | R295H, I360V, and |
| | LIHSRNVERYMVTVQAAREQLSTTADDKHOAHEIVI | L464V relative to wild- |
| | EHAADWCKLFRCDGVGYLRGEELTTYGETPDQTTI | type IsPadC (in bold) |
| | NKLVEWLEENGKKSLFWHSHMLKEDAPGLLPDGSR | 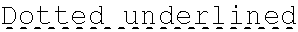 |
| | FAGLLAIPLKSDADLFSYLLLFRVAQNEVRTWAGKPE | VP16 |
| | KLSVETSTGTMVGPRKSFEAWQDEVSGKSQPWRTA | Underlined and italic: |
| | QLYAARDIARDLLIVADSMQLNLLNDQLADANENLE | T2A |
| | KLASFDDLTSGGGTSGGGGSGGGGSGGGGSGGGGSG | Double underlined: |
| | 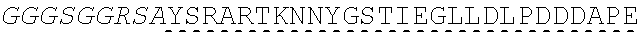 | mTagBFP2 |
| | 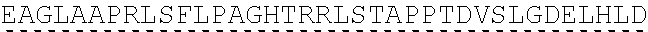 | Italic: spacing |
| | 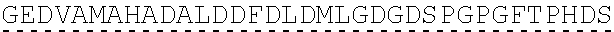 | residues/linker |
| | 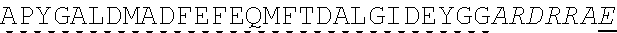 | |
| | GRGSLLTCGDVEENPGPTSSELIKENMHMKLYMEGT | |
| | VDNHHEKCTSEGEGKPYEGTQTMRIKVVEGGPLPFA | |
| | FDILATSFLYGSKTFINHTQGIPDFFKQSFPEGETWER | |
| | VTTYEDGGVLTATQDTSLODGCLIYNVKIRGVNFTS | |
| | NGPVMQKKTLGWEAFTETLYPADGGLEGRNDMAL | |
| | KLVGGSHLIANAKTTYRSKKPAKNLKMPGVYYVDY | |
| | RLERIKEANNETYVEQHEVAVARYCDLPSKLGHKLN | |
| | cDNA SEQUENCES | |
| 5 | atggcagcagatctgggtagtgatgatatcagcaaactgattgcagcatgtgatca | IsPadC-PCM (wild- |
| | agaaccgattcatattccgaatgcaattcagccgtttggtgcaatgctgattgttgaa | type iLight) |
| | aaagatacccagcagattgtttatgcaagcgcaaatagcgcagaatatttcagcgtt | 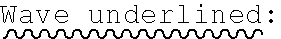 |
| | gcagataacaccattcatgaactgagcgatattaaacaggccaacattaatagcct | STOP codon |
| | gctgccggaacatctgattagcggtctggcaagcgcaattcgtgaaaatgaaccg | |
| | atttgggttgaaaccgatcgtctgagctttctgggttggcgtcatgaaaactattacat | |
| | cattgaagtggaacgctatcatgtgcagaccagcaattggtttgaaattcagtttcag | |
| | cgtgcctttcagaaactgcgtaattgcaaaacccataacgatctgattaataccctga | |
| | cccgtctgattcaagaaatcagcggttatgatcgcgtgatgatctatcagtttgatcc | |
| | ggaatggaatggtcgtgttattgcagaaagcgttcgtcagctgtttaccagcatgct | |
| | gaatcatcattttccggcaagcgatattccggcacaggcacgtgcaatgtatagcat | |
| | taatccgattcgtattatcccggatgttaatgcagaaccgcagccgctgcacatgatt | |
| | cataaaccgcagaataccgaagcagttaatctgagcagcggtgttctgcgtgcagt | |
| | tagccctctgcacatgcagtatctgcgtaattttggtgttagcgcaagcaccagcatt | |
| | ggcatttttaacgaagatgaactgtggggtattgttgcatgtcatcataccaaaccgc | |
| | gtgcaattggtcgtcgtattcgtcgtctgctggttcgtaccgttgaatttgcagcagaa | |
| | cgtctgtggctgattcatagccgtaatgttgaacgttatatggttaccgttcaggcag | |
| | cacgtgaacagctgagcaccaccgcagatgataaacattcaagccatgaaatcgt | |
| | gattgaacatgcagcagattggtgtaaactgtttcgttgtgatggtattggttatctgc | |
| | gtggtgaagaactgaccacctatggtgaaacaccggatcagaccaccattaacaa | |
| | actggttgaatggctggaagagaacggtaaaaaaagcctgttttggcatagccaca | |
| | tgctgaaagaagatgcaccgggtctgctgccggatggtagccgttttgcaggtctg | |
| | ctggcaattccgctgaaaagtgatgcagacctgtttagctatctgctgctgtttcgtgt | |
| | tgcacagaatgaagttcgtacctgggcaggtaaaccggaaaaactgagcgttgaa | |
| | accagcaccggcaccatgctgggtccgcgtaaaagttttgaagcatggcaggatg | |
| | aagttagcggtaaaagccagccgtggcgtaccgcacagctgtatgcagcacgtg | |
| | atattgcacgtgatctgctgattgtggcagatagcatgcagctgaatctgctgaatga | |
| | tcagctggcagatgcaaatgaaaatctggaaaaactggccagctttgatgatctga | |
| | 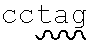 | |
| 6 | iLight (for expression in bacteria): | iLight (not codon- |
| | (SEQ ID NO: 6) | optimized) |
| | atggcagcagatctgggtagtgatgatatcagcaaactgattgcagcatgtgatca | All codons with |
| | agaaccgattcatattccgaatgcaattcagccgtttggtgcaatgctgattgttgaa | different from wild- |
| | aaagatacccagcagattgtttatgcaagcgcaaatagcgcagaatatttcagcgtt | type IsPadC-PCM |
| | gcagataacaccattcatgaactgagcgattttaaacaggccaacattaatagcctg | bases are underlined. |
| | ctgccggaacaactgattagcggtctgacaagcgcaattagtgaaaatgaaccga | Individual substitutions |
| | tttgggttgaaaccgatcgtctgagctttctgggttggcgtcatgaaaactattacatc | that lead to missense |
| | attgaagtggaacgctatcatgtgcagaccagcaattggtttgaaattcagtttcagc | mutations I68F, H80Q, |
| | gtgcctttcagaaactgcgtaattgcaaaacccataacgatctgattaataccctgac | A86T, R90S, S242C, |
| | ccgtctgattcaagaaatcagcggttatgatcgcgtgatgatctatcaatttgatccg | R274K, R295H, |
| | gaatggaatggtcgtgttattgcagaaagcgttcgtcagctgtttaccagcatgctg | I360V, and L464V are |
| | aatcatcattttccggcaagcgatattccggcacaggcacgtgcaatgtatagcatt | in bold. |
| | aatccgattcgtattatcccggatgttaatgcagaaccgcagccgctgcacatgatt | Bases that lead to |
| | cataaaccgcaaaataccgaagcagttaatctgagctgcggtgttctgcgtgcagt | silentmutations are |
| | tagccctctgcacatgcagtatctgcgtaattttggtgttagcgcaagcaccagcatt | italicized. |
| | ggcatttttaacgaagataaactgtggggtatcgttgcatgccatcataccaaaccg | 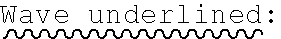 |
| | cgtgcaattggtcgtcgtattcgtcatctgctggttcgtaccgttgaatttgcagcaga | STOP codon |
| | acgtctgtggctgattcatagccgtaatgttgaacgttatatggttaccgttcaggca | |
| | gcacgtgaacagctgagcaccaccgcagatgataaacattcaagccatgaaatcg | |
| | tgattgaacatgcagcagattggtgtaaactgtttcgttgtgatggtgttggttatctgc | |
| | gtggagaagaactgaccacctatggtgaaacaccggatcagaccaccattaacaa | |
| | actggttgaatggctggaagagaacggtaaaaaaagcctgttttggcatagccaca | |
| | tgctgaaagaagatgcaccgggtctgctgccggatggtagccgttttgcaggtctg | |
| | ctggcaattccgctgaaaagtgatgcagacctgtttagctatctgctgctgtttcgtgt | |
| | ggcacagaatgaagttcgtacctgggcgggtaaaccggaaaaactgagcgttga | |
| | aaccagcactggcaccatggtgggtccgcgtaaaagttttgaagcatggcaggat | |
| | gaagttagcggtaaaagccagccgtggcgtaccgcacagctgtatgcagcacgt | |
| | gatattgcacgtgatctgctgattgtggcagatagcatgcagctgaatctgctgaat | |
| | gatcagctggcagatgcaaatgaaaatctggaaaaactggccagctttgatgatct | |
| | ga 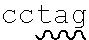 | |
| | iLight (for expression in eukaryotic cells): | iLight (codon |
| | (SEQ ID NO: 49) | optimized) |
| | atggccgccgacctgggctctgacgatatcagcaagctgatcgccgcctgcgatc | All codons that lead to |
| | aggagccaatccacatccccaatgccatccagccatttggcgccatgctgatcgtg | missense mutations |
| | gagaaggacacacagcagategtgtacgcctctgccaacagegccgagtacttc | I68F, H80Q, A86T, |
| | agcgtggccgacaataccatccacgagctgtccgatttcaagcaggccaacatca | R90S, S242C, R274K, |
| | attctctgctgcccgagcagctgatcagcggcctgacatccgccatctctgagaa | R295H, I360V, and |
| | cgagcctatctgggtggagaccgacaggctgagctttctgggctggcgccacga | L464V relative to wild- |
| | gaactactatatcatcgaggtggagagataccacgtgcagacatccaattggttcg | type IsPadC-PCM are |
| | agatccagtttcagcgggccttccagaagctgagaaactgtaagacccacaacga | in bold. |
| | tctgatcaataccctgacacggctgatccaggagatcagcggctacgacagagtg | 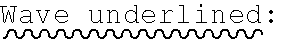 |
| | atgatctatcagttcgatcccgagtggaatggcagagtgatcgccgagagcgtga | STOP codon |
| | gacagctgtttacctccatgctgaaccaccacttcccagcctctgacatccctgcac | |
| | aggccagggccatgtacagcatcaacccaatccgcatcatccccgatgtgaatgc | |
| | cgagccccagcctctgcacatgatccacaagccacagaacacagaggccgtgaa | |
| | tctgtcctgcggcgtgctgagggccgtgtctccactgcacatgcagtatctgcgca | |
| | actttggcgtgtctgccagcacctccatcggcatcttcaatgaggacaagctgtgg | |
| | ggcatcgtggcctgtcaccacacaaagcctagggccatcggccggagaatcagg | |
| | cacctgctggtgcgcaccgtggagtttgcagcagagcgcctgtggctgatccact | |
| | ccaggaatgtggagcggtacatggtgacagtgcaggcagcccgggagcagctg | |
| | tctaccacagccgacgataagcacagctcccacgagatcgtgatcgagcacgcc | |
| | gccgactggtgcaagctgttccggtgtgatggcgtgggctacctgagaggcgag | |
| | gagctgaccacatatggcgagacccctgatcagaccacaatcaacaagctggtg | |
| | gagtggctggaggagaatggcaagaagagcctgttttggcactcccacatgctga | |
| | aggaggacgcacctggactgctgccagatggcagccggttcgcaggactgctgg | |
| | ccatcccactgaagtctgacgccgatctgtttagctacctgctgctgttcagggtgg | |
| | cacagaacgaggtgcgcacatgggcaggcaagcctgagaagctgtccgtggag | |
| | acctctacaggcaccatggtgggcccacggaagtcttttgaggcctggcaggacg | |
| | aggtgagcggcaagtcccagccttggagaaccgcacagctgtatgcagcccgg | |
| | gacatcgcccgggacctgctgatcgtggccgatagcatgcagctgaacctgctga | |
| | atgaccagctggccgatgccaacgagaatctggagaagctggcctccttcgacga | |
| | tctgacc 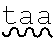 | |
| 7 | 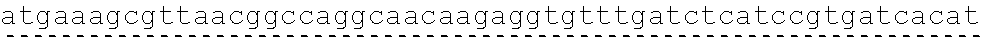 | LexA408:1-87--iLight- |
| | 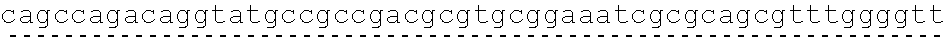 | --msfGFP |
| | 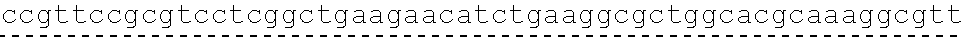 | 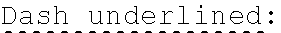 |
| | 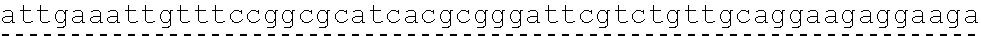 | LexA408:1-87 |
| | 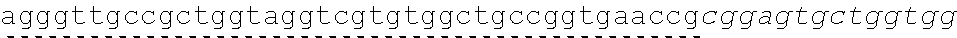 | Underlined: iLight |
| | ggcagcagatctgggtagtgatgatatcagcaaactgattgcagcatgtgatcaag | (bases that lead to |
| | aaccgattcatattccgaatgcaattcagccgtttggtgcaatgctgattgttgaaaa | following mutations, |
| | agatacccagcagattgtttatgcaagcgcaaatagcgcagaatatttcagcgttgc | I68F, H80Q, A86T, |
| | agataacaccattcatgaactgagcgattttaaacaggccaacattaatagcctgct | R90S, S242C, R274K, |
| | gccggaacaactgattagcggtctgacaagcgcaattagtgaaaatgaaccgattt | R295H, I360V, and |
| | gggttgaaaccgatcgtctgagctttctgggttggcgtcatgaaaactattacatcat | L464V relative to wild- |
| | tgaagtggaacgctatcatgtgcagaccagcaattggtttgaaattcagtttcagcgt | type IsPadC, are in |
| | gcctttcagaaactgcgtaattgcaaaacccataacgatctgattaataccctgacc | bold. |
| | cgtctgattcaagaaatcagcggttatgatcgcgtgatgatctatcaatttgatccgg | Silent mutations are in |
| | aatggaatggtcgtgttattgcagaaagcgttcgtcagctgtttaccagcatgctgaa | italic) |
| | tcatcattttccggcaagcgatattccggcacaggcacgtgcaatgtatagcattaat | Double underlined: |
| | ccgattcgtattatcccggatgttaatgcagaaccgcagccgctgcacatgattcat | msfGFP |
| | aaaccgcaaaataccgaagcagttaatctgagctgcggtgttctgcgtgcagttag | Italic: spacing |
| | ccctctgcacatgcagtatctgcgtaattttggtgttagcgcaagcaccagcattgg | residues/linker |
| | catttttaacgaagataaactgtggggtatcgttgcatgccatcataccaaaccgcgt | 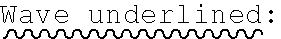 |
| | gcaattggtcgtcgtattcgtcatctgctggttcgtaccgttgaatttgcagcagaac | STOP codon |
| | gtctgtggctgattcatagccgtaatgttgaacgttatatggttaccgttcaggcagc | |
| | acgtgaacagctgagcaccaccgcagatgataaacattcaagccatgaaatcgtg | |
| | attgaacatgcagcagattggtgtaaactgtttcgttgtgatggtgttggttatctgcgt | |
| | ggagaagaactgaccacctatggtgaaacaccggatcagaccaccattaacaaa | |
| | ctggttgaatggctggaagagaacggtaaaaaaagcctgttttggcatagccacat | |
| | gctgaaagaagatgcaccgggtctgctgccggatggtagccgttttgcaggtctgc | |
| | tggcaattccgctgaaaagtgatgcagacctgtttagctatctgctgctgtttcgtgtg | |
| | gcacagaatgaagttcgtacctggggggtaaaccggaaaaactgagcgttgaa | |
| | accagcactggcaccatggtgggtccgcgtaaaagttttgaagcatggcaggatg | |
| | aagttagcggtaaaagccagccgtggcgtaccgcacagctgtatgcagcacgtg | |
| | atattgcacgtgatctgctgattgtggcagatagcatgcagctgaatctgctgaatga | |
| | tcagctggcagatgcaaatgaaaatctggaaaaactggccagctttgatgatctga | |
| | ccgtcgactccggtggtggttctggtggtggaatggtgagcaagggcgaggagc | |
| | tgttcaccggggtggtgcccatcctggtcgagctggacggcgacgtaaacggcc | |
| | acaagttcagcgtgcgcggcgagggcgagggcgatgccaccaacggcaagct | |
| | gaccctgaagttcatctgcaccaccggcaagctgcccgtgccctggcccaccctc | |
| | gtgaccaccctgacctacggcgtgcagtgcttcagccgctaccccgaccacatga | |
| | agcgccacgacttcttcaagtccgccatgcccgaaggctacgtccaggagcgca | |
| | ccatcagcttcaaggacgacggcacctacaagacccgcgccgaggtgaagttcg | |
| | agggcgacaccctggtgaaccgcatcgagctgaagggcatcgacttcaaggag | |
| | gacggcaacatcctggggcacaagctggagtacaacttcaacagccacaacgtct | |
| | atatcaccgccgacaagcagaagaacggcatcaaggccaacttcaagatccgcc | |
| | acaacgtggaggacggcagcgtgcagctcgccgaccactaccagcagaacacc | |
| | cccatcggcgacggccccgtgctgctgcccgacaaccactacctgagcacccag | |
| | tccaagctgagcaaagatcccaacgagaaacgcgatcacatggtcctgctggagt | |
| | tcgtgaccgccgccgggatcactctcggcatggacgagctgtacaagtga | |
| 8 | 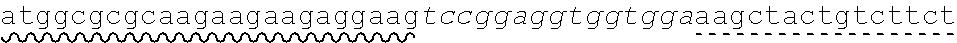 | NLS-Gal4:1-65-- |
| | 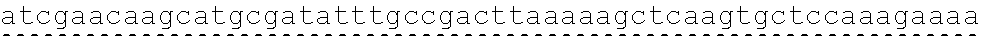 | iLight--VP16--T2A-- |
| | 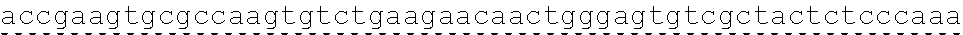 | mTagBFP2 |
| | 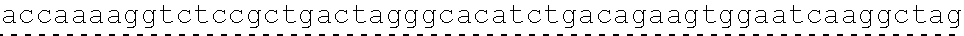 | 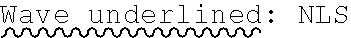 |
| | 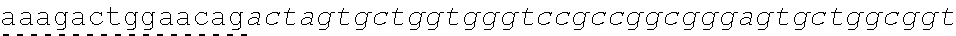 | 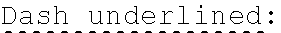 |
| | agtgccggcggtgaattcgccgccgacctgggctctgacgatatcagcaagctg | Gal4:1-65 |
| | atcgccgcctgcgatcaggagccaatccacatccccaatgccatccagccatttgg | Underlined: iLight |
| | cgccatgctgatcgtggagaaggacacacagcagatcgtgtacgcctctgccaac | (codons with |
| | agcgccgagtacttcagcgtggccgacaataccatccacgagctgtccgatttca | substitutions I68F, |
| | agcaggccaacatcaattctctgctgcccgagcagctgatcageggcctgacatc | H80Q, A86T, R90S, |
| | cgccatctctgagaacgagcctatctgggtggagaccgacaggctgagctttctg | S242C, R274K, |
| | ggctggcgccacgagaactactatatcatcgaggtggagagataccacgtgcag | R295H, I360V, and |
| | acatccaattggttcgagatccagtttcagcgggccttccagaagctgagaaactgt | L464V relative to wild- |
| | aagacccacaacgatctgatcaataccctgacacggctgatccaggagatcagcg | type IsPadC are in |
| | gctacgacagagtgatgatctatcagttcgatcccgagtggaatggcagagtgatc | bold) |
| | gccgagagcgtgagacagctgtttacctccatgctgaaccaccacttcccagcctc | 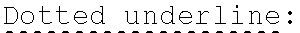 |
| | tgacatccctgcacaggccagggccatgtacagcatcaacccaatccgcatcatc | VP16 |
| | cccgatgtgaatgccgagccccagcctctgcacatgatccacaagccacagaac | Underlined and italic: |
| | acagaggccgtgaatctgtcctgcggcgtgctgagggccgtgtctccactgcaca | T2A |
| | tgcagtatctgcgcaactttggcgtgtctgccagcacctccatcggcatcttcaatga | Double underlined: |
| | ggacaagctgtggggcatcgtggcctgtcaccacacaaagcctagggccatcgg | mTagBFP2 |
| | ccggagaatcaggcacctgctggtgcgcaccgtggagtttgcagcagagcgcct | Italic: spacing |
| | gtggctgatccactccaggaatgtggagcggtacatggtgacagtgcaggcagc | residues/linker |
| | ccgggagcagctgtctaccacagccgacgataagcacagctcccacgagatcgt | 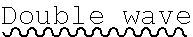 |
| | gatcgagcacgccgccgactggtgcaagctgttccggtgtgatggcgtgggctac | underlined: STOP |
| | ctgagaggcgaggagctgaccacatatggcgagacccctgatcagaccacaatc | codon |
| | aacaagctggtggagtggctggaggagaatggcaagaagagcctgttttggcact | |
| | cccacatgctgaaggaggacgcacctggactgctgccagatggcagccggttcg | |
| | caggactgctggccatcccactgaagtctgacgccgatctgtttagctacctgctgc | |
| | tgttcagggtggcacagaacgaggtgcgcacatgggcaggcaagcctgagaag | |
| | ctgtccgtggagacctctacaggcaccatggtgggcccacggaagtcttttgagg | |
| | cctggcaggacgaggtgagcggcaagtcccagccttggagaaccgcacagctg | |
| | tatgcagcccgggacatcgcccgggacctgctgatcgtggccgatagcatgcag | |
| | ctgaacctgctgaatgaccagctggccgatgccaacgagaatctggagaagctg | |
| | gcctccttcgacgatctgacctctggcggcggtaccagcgggggtggtggatcag | |
| | gtggaggaggttctggaggtggtggatcaggaggaggtggttctggaggtggtg | |
| | 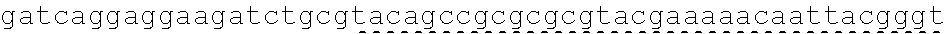 | |
| | 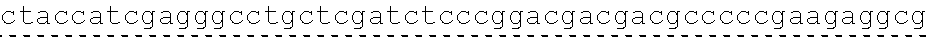 | |
| | 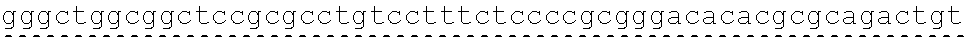 | |
| | 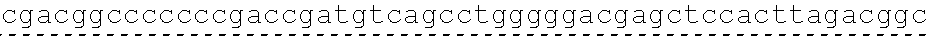 | |
| | 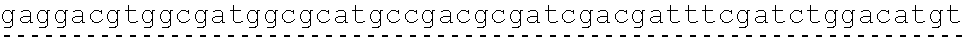 | |
| | 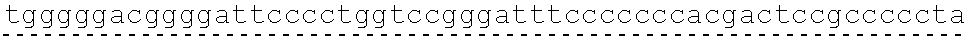 | |
| | 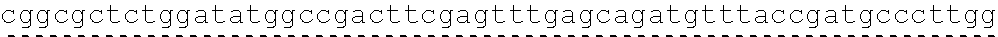 | |
| | 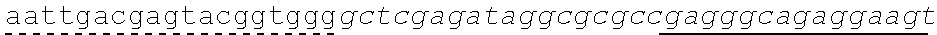 | |
| | ctgctaacatgcggtgacgtcgaggagaatcctggcccaactagtagcgagct | |
| | gattaaggagaacatgcacatgaagctgtacatggagggcaccgtggacaaccat | |
| | cacttcaagtgcacatccgagggcgaaggcaagccctacgagggcacccagac | |
| | catgagaatcaaggtggtcgagggcggccctctccccttcgccttcgacatcctgg | |
| | ctactagcttcctctacggcagcaagaccttcatcaaccacacccagggcatcccc | |
| | gacttcttcaagcagtccttccctgagggcttcacatgggagagagtcaccacata | |
| | cgaagacgggggcgtgctgaccgctacccaggacaccagcctccaggacggct | |
| | gcctcatctacaacgtcaagatcagaggggtgaacttcacatccaacggccctgtg | |
| | atgcagaagaaaacactcggctgggaggccttcaccgagacgctgtaccccgct | |
| | gacggcggcctggaaggcagaaacgacatggccctgaagctcgtgggcggga | |
| | gccatctgatcgcaaacgccaagaccacatatagatccaagaaacccgctaagaa | |
| | cctcaagatgcctggcgtctactatgtggactacagactggaaagaatcaaggag | |
| | gccaacaacgagacctacgtcgagcagcacgaggtggcagtggccagatactg | |
| | cgacctccctagcaaactggggcacaagcttaat 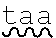 | |

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

In some embodiments, the polynucleotide may include a codon-optimized sequence. For example, the nucleotide sequence encoding the light-responsive polypeptide variant/fragment may be codon-optimized for expression in a eukaryote or eukaryotic cell. In some embodiments, the codon-optimized light-responsive polypeptide variant/fragment is codon-optimized for operability in a eukaryotic cell or organism, e.g., a yeast cell, or a mammalian cell or organism, including a mouse cell, a rat cell, and a human cell or non-human eukaryote organism.

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. Nucl. Acids Res. 28:292 (2000). Computer algorithms for codon optimizing a particular sequence for expression in a particular host cell are also available, such as Gene Forge (Aptagen; Jacobus, Pa.). As to codon usage in yeast, reference is made to the online Yeast Genome database available at http://www.yeastgenome.org/community/codonusage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al., Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

As used herein, the term “variant” refers to a first molecule that is related to a second molecule (also termed a “parent” molecule). The variant molecule can be derived from, isolated from, based on or homologous to the parent molecule. The term variant can be used to describe either polynucleotides or polypeptides.

A variant polypeptide can have an entire amino acid sequence identity with the original parent polypeptide or can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence. Polypeptide variants include polypeptides comprising the entire parent polypeptide and further comprising additional fused amino acid sequences. Polypeptide variants also include polypeptides that are portions or subsequences of the parent polypeptide. For example, unique subsequences (e.g., as determined by standard sequence comparison and alignment techniques) of the polypeptides disclosed herein are also encompassed by this disclosure. Polypeptide variants may include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide and yield functionally identical polypeptides, including additions of non-functional peptide sequence. In other aspects, the variant polypeptides change the biological activity of the parent molecule. One skilled in the art will appreciate that many variants of the disclosed polypeptides are encompassed by this disclosure. Polynucleotide or polypeptide variants can include variant molecules that alter, add or delete a small percentage of the nucleotide or amino acid positions, for example, typically less than about 10%, less than about 5%, less than 4%, less than 2% or less than 1%.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

In some embodiments, the light-responsive polypeptide comprises at least one mutation at position I68, H80, A86, R90, S242, R274, R295, 1360, or L464. In some embodiments, the at least one mutation comprises one or more substitutions selected from the group consisting of I68F, H80Q, A86T, R90S, S242C, R274K, R295H, I360V, L464V, and combinations thereof. In some embodiments, the at least one mutation comprises at least one of an I68F substitution or a conservative substitution of Phe at position 68, a R295H substitution or a conservative substitution of His at position 295, and a L464V substitution or a conservative substitution of Val at position 464. In some embodiments, the at least one mutation comprises the I68F, R295H, and L464V substitutions.

In some embodiments, the light-responsive polypeptide comprises an amino acid sequence having at least 90% sequence identity to SEQ ID NO: 2 or comprises the amino acid sequence of SEQ ID NO: 2.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The Cas protein with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine).

As used herein, the percent homology between two amino acid or nucleic acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid or nucleic acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, amino acid or nucleic acid sequences can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the disclosed polypeptides. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

In some embodiments, the light-responsive polypeptide is linked to the DNA binding domain, e.g., via a peptide linker.

In some embodiments, the DNA binding domain comprises a DNA binding motif. In some embodiments, the DNA binding motif comprises a helix-turn-helix, a homeodomain, a leucine zipper, a helix-loop-helix, or a zinc finger. In some embodiments, the DNA binding domain comprises a Gal4 DNA binding domain, a Lex-A DNA binding domain, an NF-κB DNA binding domain, a cro repressor DNA binding domain, a lac repressor DNA binding domain, a GCN4 DNA binding domain, an Opaque-2 DNA binding domain, or a TGAIa DNA binding domain.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction etc., to provide a site for coupling. Methods for conjugation are well known by persons skilled in the art and are encompassed for use in the present disclosure. Linker moieties include, but are not limited to, chemical linker moieties, or for example, a peptide linker moiety (a linker sequence).

A peptide linker can range from 2 amino acids to 60 or more amino acids, and in some embodiments, a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length. In some embodiments, a peptide linker is at least 5 amino acids, at least 6 amino acids or at least 7 amino acids in length and optionally is up to 30 amino acids, up to 40 amino acids, up to 50 amino acids or up to 60 amino acids in length. In some embodiments, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker comprises polar (e.g., serine (S)) or charged (e.g., lysine (K)) residues. In some embodiments, the linker is a flexible linker, e.g., comprising one or more glycine (G) or serine (S) residues.

Examples of flexible linkers that can be used in the fusion protein of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GnS or SGn, where n is an integer from 1 to 10, e.g., 1 2, 3, 4, 5, 6, 7, 8, 9 or 10. In one embodiment, the linker is or comprises a monomer or multimer of repeat of G4S (GGGGS; SEQ ID NO: 9), G3S (GGGS; SEQ ID NO: 10), G2S (GGS), or GS.

Polyglycine linkers can suitably be used in the fusion protein of the disclosure. In some embodiments, a peptide linker comprises two consecutive glycines (2Gly), three consecutive glycines (3Gly), four consecutive glycines (4Gly) (SEQ ID NO: 11), five consecutive glycines (5Gly) (SEQ ID NO: 12), six consecutive glycines (6Gly) (SEQ ID NO: 13), seven consecutive glycines (7Gly) (SEQ ID NO: 14), eight consecutive glycines (8Gly) (SEQ ID NO: 15) or nine consecutive glycines (9Gly) (SEQ ID NO: 16).

In some embodiments, the nucleotide sequence is operably linked to a promoter. The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions in the same reading frame.

As used herein, the term “promoter” or “regulatory sequence” refers to a nucleic acid sequence that is required for expression of a gene product operably linked to the promoter/regulatory sequence. In some instances, this sequence may be the core promoter sequence, and in other instances, this sequence may also include an enhancer sequence and other regulatory elements that are required for expression of the gene product. The promoter or regulatory sequence may, for example, be one that expresses the gene product in a tissue-specific manner. An “inducible” promoter is a nucleotide sequence that, when operably linked with a polynucleotide that encodes or specifies a gene product, causes the gene product to be produced in a cell substantially only when an inducer that corresponds to the promoter is present in the cell. The term “enhancer” as used herein refers a cis-acting regulatory sequence (e.g., 50-1,500 base pairs) is that bind one or more proteins (e.g., activator proteins or transcription factors) to increase transcriptional activation of a nucleic acid sequence. Enhancers can be positioned up to 1,000,000 base pairs upstream of the gene start site or downstream of the gene start site that they regulate. An enhancer can be positioned within an intronic region or in the exonic region of an unrelated gene.

In some embodiments, the light-responsive polypeptide, when associated with a chromophore, is capable of switching from a first state to a second state when exposed to illumination by a first wavelength and switching from the second state to the first state when exposed to illumination by a second wavelength, or returning from the second state to the first state in darkness, for example, because of thermal relaxation. In some embodiments, the chromophore is a biliverdin chromophore.

The terms “chromophore,” “photoactivating agent,” and “photoactivator” are used herein interchangeably. A chromophore means a chemical compound which, when contacted by light irradiation, is capable of absorbing the light. The chromophore readily undergoes photoexcitation and can then transfer its energy to other molecules or emit it as light.

In some embodiments, the first state is a Pr state and the second state is a Pfr state. In some embodiments, at least a portion of the light-responsive polypeptide exists in a dimeric form (e.g., homodimer) in the first state. In some embodiments, at least a portion of the light-responsive polypeptide exists in a tetrameric form (e.g., homotetramer) in the second state.

In some embodiments, at least a pair of the DNA binding domains of the tetrameric form (e.g., homotetramer) of the light-responsive polypeptide are capable of binding to a DNA recognition site.

In some embodiments, a PHY-tongue of only one protomer of the dimeric form of the light-responsive polypeptide that is constituted by two anti-parallel β-sheets in the first state is restructured to an α-helix in the second state when exposed to illumination by the first wavelength.

In some embodiments, the first wavelength and the second wavelength are in far-red and near-infrared spectrum. In some embodiments, the first wavelength is between about 600 nm and about 680 nm (e.g., 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm). In some embodiments, the is first wavelength is about 660 nm. In some embodiments, the second wavelength is between about 740 nm and about 800 nm (e.g., 740 nm, 745 nm, 750 nm, 755 nm, 760 nm, 765 nm, 770 nm, 775 nm, 780 nm, 785 nm, 790 nm, 795 nm, 800 nm). In some embodiments, the second wavelength is about 780 nm.

As used herein, “infrared” or “near-infrared” or “infrared light” or “near-infrared light” refers to electromagnetic radiation in the spectrum immediately above that of visible light, measured from the nominal edge of visible red light at 0.74 mh, and extending to 300 mh. These wavelengths correspond to a frequency range of approximately 1 to 400 THz. In particular, “near-infrared” or “near-infrared light” also refers to electromagnetic radiation measuring 0.75-1.4 m in wavelength, defined by the water absorption. “Visible light” is defined as electromagnetic radiation with wavelengths between 380 nm and 750 nm. In general, “electromagnetic radiation,” including light, is generated by the acceleration and deceleration or changes in movement (vibration) of electrically charged particles, such as parts of molecules (or adjacent atoms) with high thermal energy, or electrons in atoms (or molecules).

In some embodiments, the polynucleotide further comprises a second nucleotide sequence encoding a second light-responsive polypeptide. In some embodiments, the second light-responsive polypeptide comprises rhodopsin. Examples of rhodopsin may include a protein encoded by the NCBI reference sequence NP_0005300.1.

### B. LIGHT-RESPONSIVE POLYPEPTIDES

In another aspect, this disclosure also provides a light-responsive polypeptide comprising an amino acid sequence having at least 80% (80%, 85%, 90%, 91%, 92%, 93%, 94%, 95%, 96%, 97%, 98%, 99%) sequence identity to SEQ ID NO: 2, 3 or 4 or comprising an amino acid sequence of SEQ ID NO: 2, 3 or 4.

In some embodiments, the light-responsive polypeptide is a variant of a PCM or an IsPadC. In some embodiments, the light-responsive polypeptide comprises at least one mutation at position I68, H80, A86, R90, S242, R274, R295, 1360, or L464. In some embodiments, the at least one mutation comprises one or more substitutions selected from the group consisting of I68F, H80Q, A86T, R90S, S242C, R274K, R295H, I360V, L464V, and combinations thereof. In some embodiments, the at least one mutation comprises at least one of an I68F substitution or a conservative substitution of Phe at position 68, a R295H substitution or a conservative substitution of His at position 295, and a L464V substitution or a conservative substitution of Val at position 464. In some embodiments, the at least one mutation comprises the I68F, R295H, and L464V substitutions.

In some embodiments, the light-responsive polypeptide further comprises a DNA binding domain linked to the amino acid sequence. In some embodiments, the DNA binding domain comprises a DNA binding motif. In some embodiments, the DNA binding motif comprises a helix-turn-helix, a homeodomain, a leucine zipper, a helix-loop-helix, or a zinc finger. In some embodiments, the DNA binding domain comprises a Gal4 DNA binding domain, a Lex-A DNA binding domain, an NF-κB DNA binding domain, a cro repressor DNA binding domain, a lac repressor DNA binding domain, a GCN4 DNA binding domain, an Opaque-2 DNA binding domain, or a TGAIa DNA binding domain.

In some embodiments, the light-responsive polypeptide further comprises a DNA binding domain linked to the amino acid sequence via a linker. In some embodiments, the linker can be a peptide linker or a non-peptide linker.

A peptide linker can range from 2 amino acids to 60 or more amino acids, and in some embodiments, a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length. In some embodiments, a peptide linker is at least 5 amino acids, at least 6 amino acids or at least 7 amino acids in length and optionally is up to 30 amino acids, up to 40 amino acids, up to 50 amino acids or up to 60 amino acids in length. In some embodiments, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker comprises polar (e.g., serine (S)) or charged (e.g., lysine (K)) residues. In some embodiments, the linker is a flexible linker, e.g., comprising one or more glycine (G) or serine (S) residues.

Examples of flexible linkers that can be used in the fusion protein of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GnS or SGn, where n is an integer from 1 to 10, e.g., 1 2, 3, 4, 5, 6, 7, 8, 9 or 10. In one embodiment, the linker is or comprises a monomer or multimer of repeat of G4S (GGGGS; SEQ ID NO: 9), G3S (GGGS; SEQ ID NO: 10), G2S (GGS), or GS.

Polyglycine linkers can suitably be used in the fusion protein of the disclosure. In some embodiments, a peptide linker comprises two consecutive glycines (2Gly), three consecutive glycines (3Gly), four consecutive glycines (4Gly) (SEQ ID NO: 11), five consecutive glycines (5Gly) (SEQ ID NO: 12), six consecutive glycines (6Gly) (SEQ ID NO: 13), seven consecutive glycines (7Gly) (SEQ ID NO: 14), eight consecutive glycines (8Gly) (SEQ ID NO: 15) or nine consecutive glycines (9Gly) (SEQ ID NO: 16).

In some embodiments, the linker is a non-peptide linker. As used herein, the term “non-peptide linker” refers to a biocompatible polymer composed of two or more repeating units linked to each other, in which the repeating units are linked to each other by any non-peptide covalent bond. This non-peptidyl linker may have two ends or three ends. Examples of the non-peptidyl linker may include, without limitation, polyethylene glycol, polypropylene glycol, a copolymer of ethylene glycol with propylene glycol, polyoxyethylated polyol, polyvinyl alcohol, polysaccharide, dextran, polyvinyl ethyl ether, biodegradable polymers such as polylactic acid (PLA), and polylactic-glycolic acid (PLGA), lipid polymers, chitins, hyaluronic acid, and combinations thereof.

In some embodiments, the light-responsive polypeptide is associated with a chromophore and capable of switching from a first state to a second state when exposed to illumination by a first wavelength and switching from the second state to the first state when exposed to illumination by a second wavelength, or returning from the second state to the first state in darkness, for example, because of thermal relaxation. In some embodiments, the chromophore is a biliverdin chromophore.

In some embodiments, the first state is a Pr state, and the second state is a Pfr state. In some embodiments, at least a portion of the light-responsive polypeptide exists in a dimeric form (e.g., homodimer) in the first state. In some embodiments, at least a portion of the light-responsive polypeptide exists in a tetrameric form (e.g., homotetramer) in the second state. In some embodiments, at least a pair of the DNA binding domains of the tetrameric form (e.g., homotetramer) of the light-responsive polypeptide are capable of binding to a DNA recognition site. In some embodiments, the PHY-tongue of only one protomer of the dimeric form of the light-responsive polypeptide that is constituted by two anti-parallel β-sheets in the first state is restructured to an α-helix in the second state when exposed to illumination by the first wavelength.

In some embodiments, the first wavelength and the second wavelength are in far-red and near-infrared spectrum. In some embodiments, the first wavelength is between about 600 nm and about 680 nm (e.g., 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm). In some embodiments, the first wavelength is about 660 nm. In some embodiments, the second wavelength is between about 740 nm and about 800 nm (e.g., 740 nm, 745 nm, 750 nm, 755 nm, 760 nm, 765 nm, 770 nm, 775 nm, 780 nm, 785 nm, 790 nm, 795 nm, 800 nm). In some embodiments, the second wavelength is about 780 nm.

In some embodiments, the light-responsive polypeptide may be conjugated or linked to a detectable tag or a detectable marker (e.g., a radionuclide, a fluorescent dye). In some embodiments, the detectable tag can be an affinity tag. The term “affinity tag” as used herein relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, MBP-tag (MBP: maltose-binding protein), Alfa-tag, Sun-tag, and Moon-tag. Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of the light-responsive polypeptide. The detectable tag and the affinity tag may also be separated by one or more amino acids. In some embodiments, the detectable tag can be conjugated or linked to the light-responsive polypeptide via a cleavable element. In the context of the present disclosure, the term “cleavable element” relates to peptide sequences that are susceptible to cleavage by chemical agents or enzymatic means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

### C. VECTORS, CELLS, AND COMPOSITIONS

In another aspect, this disclosure provides a polynucleotide encoding a polypeptide described above. In some embodiments, the polypeptide can be encoded by a single nucleic acid or by a plurality (e.g., two, three, four or more) nucleic acids. The nucleic acids of the disclosure can be DNA or RNA (e.g., mRNA).

Also provided herein are vectors comprising the polynucleotides disclosed herein encoding a light-responsive polypeptide or any variant thereof. The term “vector” or “expression vector” is synonymous with “expression construct” and refers to a nucleic acid molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector may comprise an expression cassette. Expression vectors allow transcription of large amounts of stable mRNA. Once the expression vector is inside the target cell, the ribonucleic acid molecule or protein that is encoded by the gene is produced by the cellular transcription and/or translation machinery.

The vectors may comprise a polynucleotide which encodes an RNA (e.g., RNAi, ribozymes, miRNA, siRNA) that when transcribed from the polynucleotides of the vector will is result in the accumulation of light-responsive chimeric proteins on the plasma membranes of target cells. Vectors which may be used, include, without limitation, lentiviral, HSV, and adenoviral vectors. Lentiviruses include, but are not limited to HIV-1, HIV-2, SIV, FIV, and EIAV. Lentiviruses may be pseudotyped with the envelope proteins of other viruses, including, but not limited to VSV, rabies, Mo-MLV, baculovirus and Ebola. Such vectors may be prepared using standard methods in the art.

In some embodiments, the vector is a recombinant AAV vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced, and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, which contains the rep gene involved in viral replication and expression of the viral genes; and the right-hand part of the genome, which contains the cap gene encoding the capsid proteins of the virus.

The application of AAV, for example, as a vector for gene therapy, has been rapidly developed in recent years. Wild-type AAV can infect, with a comparatively high titer, dividing or non-dividing cells, or tissues of a mammal, including human, and also can integrate into human cells at a specific site (on the long arm of chromosome 19) (Kotin, R. M., et al., Proc. Natl. Acad. Sci. USA 87: 2211-2215, 1990) (Samulski, R. J, et al., EMBO J. 10: 3941-3950, 1991 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV 11, AAV12, AAV13, AAV14, AAV15, and AAV16, wherein AAV5 is originally isolated from humans (Bantel-Schaal, and H. zur Hausen. 1984. Virology 134: 52-63), while AAV1-4 and AAV6 are all found in the study of adenovirus (Ursula Bantel-Schaal, Hajo Delius and Harald zur Hausen. J. Virol. 1999, 73: 939-947).

AAV vectors may be prepared using standard methods in the art. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors,” the disclosures of which are herein incorporated by reference in their entireties. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See, e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entireties). These publications describe various AAV-derived constructs in which the rep and/or cap genes are deleted and replaced by a gene of interest and the use of these constructs for transferring the gene of interest in vitro (into cultured cells) or in vivo (directly into an organism). The replication-defective recombinant AAVs can be prepared by co-transfecting a plasmid containing the nucleic acid sequence of interest flanked by two AAV inverted terminal repeat (ITR) regions and a plasmid carrying the AAV encapsidation genes (rep and cap genes) into a cell line that is infected with a human helper virus (for example an adenovirus). The AAV recombinants that are produced are then purified by standard techniques.

In some embodiments, the vector(s) can be encapsidated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, also provided is a recombinant virus particle (recombinant because it contains a recombinant polynucleotide) comprising any of the vectors described herein. Methods of producing such particles are known in the art and are described in U.S. Pat. No. 6,596,535.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

The disclosure also provides host cells comprising a nucleic acid of the disclosure. In one embodiment, the host cells are genetically engineered to comprise one or more nucleic acids described herein. In one embodiment, the host cells are genetically engineered by using an expression cassette. The phrase “expression cassette” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In another aspect, the above-described polynucleotide, vector, polypeptide, or cell can be incorporated into compositions. The composition may further include a pharmaceutically acceptable carrier. The pharmaceutical compositions are generally formulated in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials that are capable of administration to or upon a subject without the production of undesirable physiological effects to the degree that would prohibit administration of the composition. For is example, “pharmaceutically-acceptable excipient” includes an excipient that is useful in preparing a pharmaceutical composition that is generally safe, non-toxic, and desirable and includes excipients that are acceptable for veterinary use as well as for human pharmaceutical use.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the disclosed composition, use thereof in the compositions is contemplated. In some embodiments, a second therapeutic agent, such as an anti-cancer or anti-tumor, can also be incorporated into pharmaceutical compositions.

Also provided in this disclosure is a kit comprising a polynucleotide, a vector, a polypeptide, a cell, or a composition, as described above. The components of the kit may be provided in any form, e.g., liquid, dried or lyophilized form, preferably substantially pure and/or sterile. When the components of the kit are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit. In some embodiments, the kit may further include informational materials. The informational material of the kits is not limited in its form. For example, the informational material can include information about the production of the composition, concentration, date of expiration, batch or production site information, and so forth. In addition to the composition, the kit can include other ingredients, such as a solvent or buffer, an adjuvant, a stabilizer, or a preservative.

### D. SYSTEMS AND METHODS FOR MODULATING GENE EXPRESSION

In another aspect, this disclosure further provides a system for modulating an expression level of a gene. The system comprises a polynucleotide, a vector, a host cell, or a polypeptide, as described above, wherein the DNA binding domain is capable of binding to a regulatory element of the gene. In some embodiments, the regulatory element is a promoter or an operator.

In another aspect, this disclosure additionally provides a method for modulating a gene expression level. The method comprises: (a) introducing a polynucleotide or a vector, as described above, to a cell; and (b) exposing the cell to illumination by a first wavelength and optionally exposing the cell to illumination by a second wavelength, wherein the DNA binding domain is is capable of binding to a regulatory element of the gene. In some embodiments, the regulatory element is a promoter or an operator.

Also provided in this disclosure is a method for modulating a gene expression level, comprising: (a) providing a polypeptide or a host cell, as described above; and (b) exposing the polypeptide or the host cell to illumination by a first wavelength and optionally exposing the cell to illumination by a second wavelength, wherein the DNA binding domain is capable of binding to a regulatory element of the gene.

In some embodiments, the first wavelength and the second wavelength are in far-red and near-infrared spectrum. In some embodiments, the first wavelength is between about 600 nm and about 680 nm (e.g., 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675 nm, 680 nm). In some embodiments, the first wavelength is about 660 nm. In some embodiments, the second wavelength is between about 740 nm and about 800 nm (e.g., 740 nm, 745 nm, 750 nm, 755 nm, 760 nm, 765 nm, 770 nm, 775 nm, 780 nm, 785 nm, 790 nm, 795 nm, 800 nm). In some embodiments, the second wavelength is about 780 nm.

In some embodiments, illumination or light pulses can have a duration for any of about 1 second (sec), about 2.5 sec, about 5 sec, about 7.5 sec, about 10 sec, about 25 sec, about 50 sec, about 75 sec, about 100 sec, about 250 sec, about 500 sec, about 750 sec, about 1000 sec, about 2500 sec, about 5000 sec, about 7500 sec, about 10000 sec, about 25000 sec, about 50000 sec, about 75000 sec, or about 100000 sec inclusive, including any times in between these numbers. In some embodiments, illumination or light pulses can have a light power density of any of about 0.01 mW cm⁻², 0.025 mW cm⁻², 0.05 mW cm⁻², about 0.1 mW cm², about 0.25 mW cm⁻², about 0.5 mW cm⁻², about 0.75 mW cm⁻², about 1 mW cm⁻², about 2.5 mW cm⁻², about 5 mW cm⁻², about 7.5 mW cm⁻², about 10 mW cm⁻², about 12.5 mW cm⁻², about 15 mW cm⁻², about 17.5 mW cm⁻², about 20 mW cm-2, about 25 mW cm⁻², 50 mW cm⁻², 75 mW cm⁻², or about 100 mW cm⁻² inclusive, including any values between these numbers.

In some embodiments, the method may additionally include expanding the cells in a cell culture medium following the step of introducing to the cells a polynucleotide or a vector, as described above.

The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny with the same function or biological activity as screened or selected for in the originally transformed cell are included herein.

Physical methods for introducing a polynucleotide into a host cell include calcium phosphate precipitation, lipofection, particle bombardment, microinjection, electroporation, and the like. Methods for producing cells comprising exogenous vectors and/or nucleic acids are well known in the art. See, for example, Sambrook et al. (2001, Molecular Cloning: A Laboratory Manual, Cold Spring Harbor Laboratory, New York). Chemical means for introducing a polynucleotide into a host cell include colloidal dispersion systems, such as macromolecule complexes, nanocapsules, microspheres, beads, and lipid-based systems, including oil-in-water emulsions, micelles, mixed micelles, and liposomes. An exemplary colloidal system for use as an in vitro and in vivo release vehicle is a liposome (e.g., an artificial membrane vesicle).

In the case in which a non-viral delivery system is used, an exemplary delivery vehicle is a liposome. Lipid formulations can be used for the introduction of nucleic acids into a host cell (in vitro, ex vivo, or in vivo). In one example, the nucleic acid may be associated with a lipid. The nucleic acid associated with a lipid may be encapsulated in the aqueous interior of a liposome, interspersed within the lipid bilayer of a liposome, bound to a liposome via a binding molecule that is associated with both the liposome and the oligonucleotide, entrapped in a liposome, in a complex with a liposome, dispersed in a solution containing a lipid, mixed with a lipid, combined with a lipid, contained as a suspension in a lipid, content or in a complex with a micelle, or is associated otherwise with a lipid. The compositions associated with lipids, lipids/DNA or lipids/expression vector are not limited to any particular structure in solution. For example, they can be present in a bilayer structure, as micelles, or with a “collapsed” structure. They can also be simply interspersed in a solution, possibly forming aggregates that are not uniform in size or shape. Lipids are fatty substances that can be natural or synthetic lipids. For example, lipids include fatty droplets that occur naturally in the cytoplasm as well as the class of compounds containing long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; Dicetylphosphate (“DCP”) can be obtained from K & K Laboratories (Plainview, NY); Cholesterol (“Choi”) can be obtained from Calbiochem-Behring; dimyristyl phosphatidylglycerol (“DMPG”) and other lipids can be obtained from Avanti Polar Lipids, Inc. (Birmingham, AL). Lipid stock solutions in chloroform or chloroform/methanol can be stored at about −20° C. Chloroform is used as the sole solvent since it evaporates more easily than methanol. “Liposome” is a generic term that encompasses a variety of unique and multilamellar lipid vehicles formed by the generation of bilayers or closed lipid aggregates. Liposomes can be characterized as having vesicular structures with a bilayer membrane of phospholipids and an internal aqueous medium. Multilamellar liposomes have multiple layers of lipids separated by an aqueous medium. They form spontaneously when phospholipids are suspended in an excess of aqueous solution. The lipid components undergo self-rearrangement before the formation of closed structures and trap dissolved water and solutes between the lipid bilayers (Ghosh et al., 1991 Glycobiology 5: 505-10). However, compositions that have different structures in solution than the normal vesicular structure are also included. For example, lipids can assume a micellar structure or simply exist as nonuniform aggregates of lipid molecules. Lipofectamine-nucleic acid complexes are also contemplated.

Regardless of the method used to introduce exogenous nucleic acids into a host cell, the presence of the recombinant DNA sequence in the host cell can be confirmed by a series of tests. Such assays include, for example, “molecular biology” assays well known to those skilled in the art, such as Southern and Northern blot, RT-PCR and PCR; biochemical assays, such as the detection of the presence or absence of a particular peptide, for example, by immunological means is (ELISA and Western blot) or by assays described herein to identify agents that are within the scope of the disclosure.

### E. ADDITIONAL DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “recombinant” refers to a cell, microorganism, nucleic acid molecule or vector that has been modified by the introduction of an exogenous nucleic acid molecule or has controlled expression of an endogenous nucleic acid molecule or gene. Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule. Or other functional disruption of, or functional addition to, the genetic is material of the cell. Exemplary modifications include modifications in the coding region of a heterologous or homologous polypeptide derived from the reference or parent molecule or a functional fragment thereof.

The term “chimeric” or “heterologous” refers to two components that are defined by structures derived from different sources or progenitor sequences. For example, where “heterologous” is used in the context of a chimeric polypeptide, the chimeric polypeptide can include operably linked amino acid sequences that can be derived from different polypeptides of different phylogenic groupings.

As used herein, the term “pharmaceutical composition” refers to a mixture of at least one compound useful within the disclosure with other chemical components, such as carriers, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound and are physiologically acceptable to the subject. Supplementary active compounds may also be incorporated into the compositions.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word is “substantially” may be omitted from the definition of the disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 4%1, 3%1, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### F. EXAMPLES

### Example 1

This example describes the materials and methods used in the subsequent EXAMPLES.

### Design of Bacterial and Mammalian Plasmids

An IsPadC gene was kindly provided by A. Winkler (Graz University of Technology, Austria) (Gourinchas, G. et al. Science Advances 3, e1602498 (2017)). A transcription activating domain of transactivating tegument protein VP16 from Herpes simplex and Gal4(1-65) DNA binding domain were PCR amplified from a pGV-2ER plasmid (Systasy). ARLuc8 gene encoding modified luciferase from Renilla reniformis was PCR amplified from a Nano-lantern/pcDNA3 plasmid (Addgene #51970). A SEAP gene was PCR amplified from a pKM006 plasmid kindly provided by W. Weber (University of Freiburg, Germany) (Müller, K. et al. Nucleic Acids Research 41: e77-e77 (2013)).

Reporter plasmid for screening and selection of IsPadC-PCM mutants was based on pLEVI(408)-ColE plasmid (Chen, X. et al. Cell Res 26, 854-857 (2016)). The pLEVI(408)-ColE plasmid was kindly provided by Y. Yang (East China University of Science and Technology, China) (Chen, X. et al. Cell Research 26, 854-857 (2016)). Using quick-change mutagenesis nucleotide sequence encoding, VVD was substituted with sites for SacII and SalI endonucleases. Next, SAGG-IsPadC-PCM (1-532 amino acids) was cloned using SacII and SalI restriction sites, and 2×(SGGG)-msfGFP was cloned using SalI and EcoRI restriction sites resulting in plasmid encoding LexA408-DBD(1-87)-SAGG-IsPadC-PCM(1-532)-2×(SGGG)-msfGFP. pWA23h plasmid encoding heme oxygenase for biliverdin (BV) synthesis in E. coli was modified to provide an expression of heme oxygenase under control of the constitutively active promoter. The rhamnose-inducible promoter of pWA23h was substituted with a constitutively active β-lactamase promoter from the pUC19 plasmid, resulting in a pWA23h-bla plasmid.

The reporter plasmids pG12-SEAP and pG12-Rluc8 were obtained by cloning of the SEAP and Rluc8 genes, respectively, by AgeI and NotI sites, into a pG12 plasmid synthesized by GeneScript. Plasmids encoding a PiggyBac transposase pRP[Exp]-mCherry-CAG>hyPBase (VectorBuilder #VB160216-10057) and transposon bearing plasmid pQP-Select were kindly provided by T. Redchuk (University of Helsinki, Finland) (Redchuk, T. A. et al. Nat Chem Biol 13, 633-639 (2017)). These plasmids were used to develop a stable preclonal cell mixture of HeLa cells.

A modified pBAD/His-B (Life Technologies-Invitrogen) vector with a shorter linker between the N-terminal polyhistidine tag and the gene of interest was used for bacterial expression of the iLight protein.

Plasmid for expression of the optogenetic system in mammalian cells was based on the pEGFP-N1 vector with truncated CMVd1 promoter. EGFP was substituted with a nucleotide sequence encoding T2A-mTagBFP2 using XhoI and XmaI restriction sites. Nucleotide sequence encoding NLS-SGGGG-Gal4(1-65)-4×(SAGG)-iLight(human codon-optimized)-5×(SGGGG)-VP16 was synthesized by GenScript and cloned using NheI and XhoI restriction sites into pT2A-mTagBFP2-N1 vector.

To transduce neurons, plasmids were created for packaging of the nucleotide sequences encoding optogenetic system and reporter into AAV. The pAAV-CW3SL-EGFP plasmid (Addgene #61463) was used as a backbone. EGFP was replaced with nucleotide sequence encoding NLS-SGGGG-Gal4(1-65)-4×(SAGG)-iLight (human codon-optimized)-5×(SGGGG)-VP16 for optogenetic system. To construct the reporter plasmid, the CaMKII promoter and EGFP were replaced with the nucleotide sequence of the minimal promoter with 12 upstream activation sequences followed by the nucleotide sequence encoding CheRiff-T2A-mCherry.

The plasmids designed in this study are summarized in Table 3. The oligonucleotide primers used in this study are summarized in Table 4.

### Mutagenesis and Directed Molecular Evolution

Random mutagenesis of IsPadC-PCM (1-532 amino acids) was performed with a GeneMorph II random mutagenesis kit (Stratagene) using conditions that resulted in the mutation frequency of up to 16 mutations per 10³ base pairs. After mutagenesis, a mixture of mutated genes was cloned into pLEVI(408)-ColE-msfGFP plasmid using SacII and SalI restriction sites and electroporated into TOP10 host cells (Invitrogen) containing the pWA23h-bla plasmid facilitating BV synthesis. Typical mutant libraries consisted of more than 10⁶ independent clones. For flow cytometry enrichment of the libraries, the TOP10 cells were grown overnight at 37° C. in LB medium supplemented with spectinomycin and kanamycin in darkness. Bacterial cells were washed with phosphate-buffered saline (PBS) and diluted with PBS to an optical density of 0.03 at 600 nm. The libraries were enriched with FACSAria (BD Biosciences, software v.8.0.1) fluorescence-activated cell sorter using 488 nm and 561 nm lasers for excitation and 530/30 nm and 610/20 nm emission filters for the selection of msfGFP and mCherry double-positive cells. The 5×10⁵ bacterial cells were rescued in SOC medium at 37° C. for 1 h and then grown in LB medium supplemented with spectinomycin and kanamycin in darkness. The cells after enrichment were grown in darkness to an optical density of 0.4 at 600 nm. After 200-fold dilution in LB medium supplemented with spectinomycin and kanamycin cells were grown at 37° C. for 16 h under 660/15 nm light at 0.25 mW cm⁻². Bacterial cells were washed with phosphate-buffered saline (PBS) and diluted with PBS to an optical density of 0.03 at 600 nm. The msfGFP positive and mCherry negative cells were collected using FACSAria fluorescence-activated cell sorter using 488 nm and 561 nm lasers for excitation and 530/30 nm 610/20 nm emission filters. The 1×10⁵ collected bacterial cells were rescued in SOC medium at 37° C. for 1 h and then grown on LB/spectinomycin/kanamycin Petri dishes at 37° C. in darkness. After 10 h of cultivation, each dish was replicated on two dishes using a replica-plating tool (Cole-Parmer). Then dishes were cultivated overnight at 37° C. in the darkness and under 660/20 nm light at 0.25 mW cm-2.

Screening for mutants on Petri dishes with a decreased level of mCherry expression under 660/15 nm illumination was performed with a Leica MZ16F fluorescence stereomicroscope equipped with 480/30 nm and 570/30 nm excitation filters and 530/40 nm and 615/40 nm emission filters (Chroma). Images of two replica dishes grown in different conditions (darkness and under 660/20 nm illumination) were aligned using Template Matching and Slice Alignment ImageJ plugin, and colonies with the highest ratio of darkness/illumination mCherry signal were selected for the next round of mutagenesis.

Characterization of mCherry Expression Repression by IsPadC-PCM Mutants in Bacteria

Unless stated otherwise, all experiments were carried out in the E. coli strain TOP10 containing the pWA23h-bla plasmid facilitating biliverdin synthesis. The cells from frozen stock or bacterial streak bearing pLEVI(408)-ColE-IsPadC-PCM variant-msfGFP were grown at 37° C. in LB medium supplemented with spectinomycin and kanamycin under 660/20 nm light at 0.25 mW cm⁻² until an optical density of 0.2-0.3 at 600 nm. 2 ml of each bacterial culture diluted to an optical density of 0.002 at 600 nm were transferred to new 15 ml tubes and were cultivated at 37° C. in darkness or under illumination. After overnight cultivation, the mCherry fluorescence signal of bacteria grown in darkness or under illumination was measured using FACS or spectrofluorimeter. For FACS analysis, bacterial cells were washed with PBS and diluted with PBS to an optical density of 0.03 at 600 nm and analyzed using FACSAria (BD Biosciences) fluorescence-activated cell sorter equipped with 561 nm laser for excitation and 610/20 nm emission filter. Bacterial cells were washed with PBS and diluted with PBS to an optical density of 0.1 at 600 nm to measure mCherry signal in bacterial suspension using excitation 530 nm, emission 560-750 nm with a FluoroMax-3 spectrofluorometer (Horiba/Jobin Yvon).

To perform time-course illumination and single-point mutation analysis, bacteria were incubated in LB liquid medium supplemented with spectinomycin and kanamycin in darkness overnight. The next day, the cell suspension was transferred on LB plates with the same antibiotics. Plates were dried and immediately transferred to a 37° C. incubator either fully protected from light or illuminated with 660/15 nm LED (1 mW cm⁻²) using 30 sec on and 3 min darkness cycles. After 24 h, plates were imaged with Leica MZ16F fluorescence stereomicroscope as described above. Immediately after imaging, the bacterial cells were resuspended in ice-cold PBS for flow cytometry analysis. Flow cytometry was performed using an LSRII flow analyzer (BD Biosciences) equipped with 488 nm and 561 nm lasers for excitation and 530/40 nm and 610/20 nm emission filters, respectively. Typically 100,000 GFP-positive single cells were analyzed. To quantify cell fluorescence, a mean fluorescent intensity in the red channel was divided by the mean fluorescence intensity of the same population in the green channel.

Photochemical and Biochemical Characterizations of iLight

For bacterial expression of the iLight, its nucleotide sequence was sub-cloned into a pBAD/His-D vector using KpnI and EcoRI restriction sites. Protein with 6× polyhistidine tags on the N-terminus was expressed in BL21-AI bacteria (ThermoFisher Scientific, #C607003) containing the pWA23h plasmid for rhamnose inducible BV synthesis. The bacteria were grown in LB medium supplemented with ampicillin, kanamycin, 0.02% rhamnose for 6-8 h at 37° C. followed by induction of the protein expression by adding 0.1% arabinose and cultivation for 12 h at 37° C. and 24 h at 18° C. Protein was purified using Ni-NTA agarose (Qiagen) according to the manufacturer's protocol with minor modification. In elution buffer, 400 mM imidazole was substituted with 100 mM EDTA. After elution, the buffer was exchanged using a PD10 desalting column (GE Healthcare) or Amicon Ultra-15 centrifugal filter units (Millipore) if the additional concentration was required.

For absorbance measurements, a U-2000 spectrophotometer (Hitachi) was used. A photoconversion of the iLight variant containing proteins was performed with 660/15 nm and 780/30 nm custom assemble LED sources in quartz microcuvette (Starna Cells). A determination of action spectrum was performed by measurement of changing in absorbance of Pr state of iLight variant at 704 nm upon illumination with photoconversion light. As a source of light, the FluoroMax-3 spectrofluorometer was used, and the illumination time was normalized to the power of activation. A half-time of Pr→Pfr and Pfr→Pr transition was measured by registering absorbance at 704 nm while illuminating with 660/15 nm and 780/30 nm light, respectively. All spectroscopic measurements were performed in PBS at room temperature.

For native PAGE, proteins were diluted to the concentration of 2 mg/mL in 20 mM HEPES pH 7.7, 300 mM NaCl buffer and illuminated either with 660/15 nm or 780/30 nm light at 2 mW/cm² intensity for 0.5 h. 20 μg of protein samples were diluted in 2× loading buffer (125 mM Tris-HCl pH 6.8, 0.004% bromophenol blue, 2% glycerol) and immediately loaded on 4-20% gradient gel (BioRad). After 2 h of run in 1× Tris/Glycine running buffer without SDS, the gel was washed and incubated in 1 mM ZnCl₂ solution for 1 h, imaged for zinc-dependent fluorescence excited with UV light, after then stained with GelCode blue protein stain (BioRad).

Size exclusion liquid chromatography of the Ni-NTA purified proteins was performed in darkness using HiLoad 16/600 Superdex 200 column (GE Healthcare) at a flow rate of 1 ml/min. The column was equilibrated with 10 mM HEPES buffer pH 7.4 containing 150 mM NaCl, 10% glycerol, 50 μM EDTA, 1 mM DTT, 0.2 mM PMSF, 0.01% EP-40 and 0.2 mM benzodiazepine. The column was calibrated with Bio-Rad gel filtration standards. The proteins were diluted to the concentration of 1.9 mg ml⁻¹ in 20 mM HEPES pH 7.7, 300 mM NaCl buffer and illuminated either with 660/15 nm or 780/30 nm light at 2 mW cm⁻² intensity for 0.5 h before applying to the column.

### Mammalian Cell Culture

HeLa cells were grown in DMEM medium supplemented with 10% FBS, penicillin-streptomycin mixture (all from Life Technologies-Invitrogen) at 37° C. in 5% C02. Transient cell transfections were performed using an Effectene reagent (Qiagen).

Preclonal mixtures of HeLa cells were obtained using the plasmid-based PiggyBac transposon system. To this end, the desired for integration into genome sequences were cloned into the transposon bearing plasmids pQP-Select and co-transfected with a plasmid encoding a hyperactive PiggyBac transposase. Cells were further selected with 700 μg ml⁻¹ of G418 antibiotic for two weeks and enriched with FACSAria (BD Biosciences) fluorescence-activated cell sorter using 407 nm laser for excitation and 450/50 nm emission filter for selection of mTagBFP2 positive cells, resulting in the preclonal HeLa cell mixtures expressing NLS-Gal4(1-65)-iLight-VP16-T2A-mTagBFP2 under control of CMVd1 promoter.

To study transcription activation using iLight optogenetic system, HeLa cells stably expressing NLS-Gal4(1-65)-iLight-VP16-T2A-mTagBFP2 were transfected with a pG12-SEAP reporter plasmid and illuminated with 660/15 nm light.

### Secreted Alkaline Phosphatase Assay

For SEAP detection in culture media, a Great EscAPe SEAP Fluorescence Assay kit (Clontech) was used. 25 μl aliquots of cell culture media from wells of a 24-well plate were collected at each time point and stored at −20° C. The fluorescence intensity of the SEAP reaction product was measured using the SpectraMax M2 plate reader (Molecular Devices).

### AAV Production

High-titer AAV particles were obtained as described here (Challis, R. C. et al. Nat Protoc 14, 379-414 (2019)). Briefly, plasmid DNA for AAV production was purified with NucleoBond Xtra Maxi EF kit (Macherey-Nagel), and AAV-293 cells (Agilent Technologies) were co-transfected with AAV genome plasmid, pAAV-G12-mCherry-T2A-CheRiff (encoding reporters) or pAAV-CaMKII-Gal4-iLight-VP16 (encoding optogenetic system), AVV capsid plasmid pUCmini-iCAP-PHP.eB and pHelper using polyethyleneimine (PEI, Santa Cruz). Cell media was collected 72 h after transfection. 120 h after transfection, cells and media were collected and combined with media collected at 72 h. Cells were harvested by centrifugation and then lysed with salt-active nuclease (HL-SAN, Arcticzyme). 8% PEG was added to media, incubated 2 h on ice and then pelleted. PEG pellet was treated with SAN and combined with lysed cells. The cell suspension was clarified by centrifugation. The supernatant was applied on iodixanol gradient and subjected to ultracentrifugation 2 h 25 min at 350,000 g. Virus fraction was collected, washed, and enriched on Amicon-15 100,000 MWCO centrifuge device. Virus titer was defined by qPCR. An aliquot of the virus was consequently treated with DNAse I and proteinase K and then used as a template for qPCR. A NheI digested, pAAV-G12-mCherry-T2A-CheRiff or pAAV-CaMKII-Gal4-iLight-VP16 plasmid with known concentration was used as a reference.

### Isolation and Viral Transduction of Primary Mouse Neurons

Neurons were isolated from hippocampi of postnatal (P0-P1) Swiss-Webster mice using the protocol from Beaudoin et al. (Beaudoin, G. M., et al. Nat Protoc 7, 1741-1754 (2012)) and cultured in Neurobasal Plus Medium with B-27 Plus Supplement (Gibco), additional 1 mM GlutaMAX (Gibco), 100 U/ml penicillin, and 100 μg/ml streptomycin, on poly-D-lysine (EMD Millipore) coated glass coverslips (thickness 0.13 to 0.17 mm, diameter 12 mm, ThermoFisher Scientific). Cell density was ˜70,000 cells per coverslip. Half of the medium was exchanged twice a week. Neurons were transduced with AAVs on DIV7 (10′ viral genomes per well, medium volume 0.5 ml, in 24 wells plate). After transduction 2 μM of BV was added.

### Characterization of the Optogenetic System in Neurons

Neurons were transferred from darkness to 660 nm light (30 s On, 180 s Off cycle, 0.5 mW cm⁻²) on DIV12 (5 days after transduction) and recorded on DIV17 (10 days after transduction). Fluorescence of mCherry in neurons was measured using Olympus IX81 inverted microscope controlled by Micro-Manager 1.3 (Vale Lab, UCSF) and Matlab R2018b (MathWorks). The microscope was equipped with 585 nm LED (Mightex Systems), 650/45 nm excitation filter, 695LP dichroic mirror, 725/50 nm emission filter (Chroma), LUCPlanFLN 20×/0.45NA objective (Olympus), Orca Flash 4.0LT camera, and HCImage software (Hamamatsu).

For the characterization of CheRiff expression, the steady-state ionic photocurrents were measured. It was assumed that the channelrhodopsin expression level is proportional to the number of functional channelrhodopsin molecules per unit of cell membrane area and divided the photocurrent value (measured in pA) by the respective value of cell membrane capacitance (measured in pF and presumably proportional to cell membrane area). The neurons were patch-clamped in whole-cell configuration.

Patch pipettes were pulled from borosilicate glass with filament (O.D. 1.5 mm, I.D. 0.86 mm, Sutter Instruments) to resistance of 3-5 MΩ on β-1000 puller (Sutter Instruments). External bath solution contained 125 mM NaCl, 2.5 mM KCl, 1 mM MgC₂, 10 mM HEPES, 3 mM CaCl₂), 30 mM glucose, pH 7.3, 305-307 mOsm. Internal solution contained 125 mM potassium gluconate, 8 mM NaCl, 0.6 mM MgCl₂, 0.1 mM CalCl₂, 1 mM EGTA, 4 mM MgATP, 0.4 mM NaGTP, 10 mM HEPES, pH 7.3, 294-297 mOsm. Positive pressure (30-45 mbar) was maintained while the pipette was approaching a cell. Gigaseal was established using 30-100 mbar negative pressure. For breaking the patch of the membrane a pulse of −100 to −150 mbar negative pressure (duration ˜50 ms) was applied concurrently with a single 1 V, 0.2 ms voltage pulse (‘zap’). Voltage and current values were recorded and digitized with Intan CLAMP Patch Clamp Amplifier System at 50 kHz (Intan Technologies) (Harrison, R. R. et al. J Neurophysiol 113, 1275-1282 (2015)). Cell membrane capacitance was estimated by delivering square voltage pulses (10 mV, 50 ms duration, 50 Hz, holding voltage −70 mV), measuring resulting currents, and fitting an exponential curve to the current trace. The estimation was performed automatically by Clamp UI software v.1.4.0 (Intan Technologies). Photocurrents were recorded in voltage clamp mode (˜70 mV) while flashes of green light (duration 1 s, 505 nm LED, Mightex Systems, with 510/20 nm filter) were delivered. Values of resulting steady-state photocurrent were measured and divided by values of membrane capacitance to normalize photocurrents by cell membrane area. The timing of light pulses was controlled with Master-8 pulse stimulator (AMPI, Israel). Neuron images and traces of current and voltage were processed in Matlab R2018b (MathWorks).

### Hydrodynamic Transfection of Liver in Mice

The Swiss Webster 2-3-month-old female mice (National Cancer Institute, NIH) with body weight of 22-25 g were used for delivery of plasmids encoding optogenetic system and reporter protein into the liver by hydrodynamic transfection. 10 μg of the pCMVd1-NLS-Gal4(1-65)-iLight-VP16-T2A-mTagBFP2 plasmid and 50 μg of the pG12-Rluc8 reporter plasmid in 1.5 ml of PBS were intravenously injected through a tail vein. The mice were placed in the cage without bedding and illuminated from the bottom with the 660/20 nm LED array; control animals were kept in the darkness. Intensity of activation light was 3.2 mW cm². For better illumination and imaging, the belly fur was removed using a depilatory cream.

Animals were continuously illuminated or kept in darkness for 72 h, and every 12 h were released and fed for 30 min. Every 24 h after the hydrodynamic transfection, animals were imaged using an IVIS Spectrum instrument (Perkin Elmer/Caliper Life Sciences) in bioluminescence mode with an open emission filter. Throughout the imaging, animals were maintained under anesthesia with 1.5% vaporized isoflurane. Before imaging, 80 μg of Inject-A-Lume CTZ native (NanoLight Technology) were intravenously injected through a retro-orbital vein.

Data were analyzed using Living Image v.3.0 software (Perkin Elmer/Caliper Life Sciences). Specifically, the average signal from each animal was calculated from a region of interest located over the liver of the animal; and each region of interest was the same size.

All animal experiments were performed in an AAALAC-approved facility using protocols approved by the Albert Einstein College of Medicine Animal Usage Committee.

TABLE 2
Mutations in the IsPadC-PCM variants
acquired during molecular evolution.

| IsPadC-PCM | Amino acid mutations | | |
|---|---|---|---|
| variants | PAS domain | GAF domain | PHY domain |
| 1.3 | | E274K, R295H | I360V, L464M |
| 2.24 | | T265I, E274K, R295H | I360V, L464M |
| iLight | I68F, H80Q, | S242C, E274K, R295H | I360V, L464V |
| | A86T, R90S | | |

TABLE 3
List of the major plasmids designed in this study.

| Plasmid | Backbone | Promoter | Insert |
|---|---|---|---|
| pLEVI(408)-ColE- | pLEVI(408)-ColE | J23116 promoter for | iLight-msfGFP |
| iLight-msfGFP | | iLight construct, and | |
| | | ColE with a LexA408 | |
| | | operator for mCherry | |
| | | reporter | |
| pWA23h-bla | pWA23h | β-lactamase promoter | heme oxygenase |
| pBAD/His-D-iLight | modified | araBAD | iLight |
| | pBAD/His-B | | |
| pG12-SEAP | pG12 | 12xUAS with TATA- | SEAP |
| | | box minimal promoter | |
| pG12-Rluc8 | pG12 | 12xUAS with TATA- | RLuc8 |
| | | box minimal promoter | |
| pCMVd1-NLS-Gal4- | pQP-Select | CMVd1 | NLS-Gal4(1-65)- |
| iLight-VP16-T2A- | | | iLight(human codon |
| mTagBFP2 | | | optimized)-VP16- |
| | | | T2A-mTagBFP2 |
| pAAV-CaMKII-Gal4- | pAAV-CW3SL- | CaMKII | NLS-Gal4(1-65)- |
| iLight-VP16 | EGFP | | iLight(human codon |
| | | | optimized)-VP16 |

TABLE 4
List of the major oligonucleotide
primers used in this example.

| | | | SEQ |
|---|---|---|---|
| | | Sequence | ID |
| Purpose | Primer | (5′-3′) | NO |
| Cloning of IsPadC- | forward | ttggtaccgcagcag | 17 |
| PCM or iLight into | | atctgggtagtgatg | |
| pBAD/His-D | | at | |
| | reverse | ttgaattcctaggtc | 18 |
| | | agatcatcaaagctg | |
| | | gccag | |
| Cloning of IsPadC- | forward | aaccgcggagtgctg | 19 |
| PCM or iLight with | | gggggcagcagatct | |
| linker into | | gggtagtgatgatat | |
| pLEVI(408)-ColE | | c | |
| | reverse | aagaattcgctgtcg | 20 |
| | | acggtcagatcatca | |
| | | aagctggccag | |
| Cloning of msfGFP | forward | gaccgtcgactccgg | 21 |
| with linker into | | tggtggttctggtgg | |
| pLEVI(408)-ColE | | tggaatggtgagcaa | |
| | | gggcgagg | |
| | reverse | gaattctcacttgta | 22 |
| | | cagctcgtccatgcc | |
| | | gagagtgatccc | |
| Cloning of SEAP | forward | cgtcaccggtcgcca | 23 |
| into pG12 | | ccatgctgctgctgc | |
| | | tgctgctgc | |
| | reverse | catcgcggccgctta | 24 |
| | | acccgggtgcgcggc | |
| | | gtcg | |
| Cloning of Rluc8 | forward | cgtaccggtcgccac | 25 |
| into pG12 | | catggcttccaaggt | |
| | | gtacgaccccgagc | |
| | reverse | ctggcggccgcttac | 26 |
| | | tgctcgttcttcagc | |
| | | actc | |
| Cloning of iLight | forward | ttgaattcgccgccg | 27 |
| into mammalian | | acctgggctctgacg | |
| vector. | | at | |
| | reverse | gctggtaccgccgcc | 28 |
| | | agaggtcagatcgtc | |
| | | gaaggaggccag | |
| Cloning of CheRiff | forward | ttggtaccggtcgcc | 29 |
| with Kozak into | | accatgggcggagct | |
| pAAV-CW3SL-EGFP. | | cctgctc | |
| | reverse | atctcgagccacgtt | 30 |
| | | gatgtcgatctggtc | |
| | | cag | |
| Introduction of | forward | gataacaccattcat | 31 |
| F68I amino acid | | gaactgagcgatatt | |
| substitution into | | aaacaggccaacatt | |
| iLight. | | aatagcctgc | |
| | reverse | gcaggctattaatgt | 32 |
| | | tggcctgtttaatat | |
| | | cgctcagttcatgaa | |
| | | tggtgttatc | |
| Introduction of | forward | cattaatagcctgct | 33 |
| Q80H amino acid | | gccggaacatctgat | |
| substitution into | | tagcggtctgacaag | |
| iLight. | | cgc | |
| | reverse | gcgcttgtcagaccg | 34 |
| | | ctaatcagatgttcc | |
| | | ggcagcaggctatta | |
| | | atg | |
| Introduction of | forward | cggaacaactgatta | 35 |
| T86A amino acid | | gcggtctggcaagcg | |
| substitution into | | caattagtgaaaatg | |
| iLight. | | aacc | |
| | reverse | ggttcattttcacta | 36 |
| | | attgcgcttgccaga | |
| | | ccgctaatcagttgt | |
| | | tccg | |
| Introduction of | forward | gcggtctgacaagcg | 37 |
| S90R amino acid | | caattcgtgaaaatg | |
| substitution into | | aaccgatttgggttg | |
| iLight. | | | |
| | reverse | caacccaaatcggtt | 38 |
| | | cattttcacgaattg | |
| | | cgcttgtcagaccgc | |
| Introduction of | forward | caaaataccgaagca | 39 |
| C242S amino acid | | gttaatctgagcagc | |
| substitution into | | ggtgttctgcgtgca | |
| iLight. | | gttag | |
| | reverse | ctaactgcacgcaga | 40 |
| | | acaccgctgctcaga | |
| | | ttaactgcttcggta | |
| | | ttttg | |
| Introduction of | forward | cagcattggcatttt | 41 |
| K274E amino acid | | taacgaagatgaact | |
| substitution into | | gtggggtatcgttgc | |
| iLight. | | atg | |
| | reverse | catgcaacgataccc | 42 |
| | | cacagttcatcttcg | |
| | | ttaaaaatgccaatg | |
| | | ctg | |
| Introduction of | forward | gcaattggtcgtcgt | 43 |
| H295R amino acid | | attcgtcgtctgctg | |
| substitution into | | gttcgtaccgttgaa | |
| iLight. | | tttg | |
| | reverse | caaattcaacggtac | 44 |
| | | gaaccagcagacgac | |
| | | gaatacgacgaccaa | |
| | | ttgc | |
| Introduction of | forward | ggtgtaaactgtttc | 45 |
| V360I amino acid | | gttgtgatggtattg | |
| substitution into | | gttatctgcgtggag | |
| iLight. | | aagaac | |
| | reverse | gttcttctccacgca | 46 |
| | | gataaccaataccat | |
| | | cacaacgaaacagtt | |
| | | tacacc | |
| Introduction of | forward | gaaaccagcactggc | 47 |
| V464L amino acid | | accatgctgggtccg | |
| substitution into | | cgtaaaagttttg | |
| iLight. | | | |
| | reverse | caaaacttttacgcg | 48 |
| | | gacccagcatggtgc | |
| | | cagtgctggtttc | |

### Example 2

### Molecular Evolution of IsPadC-PCM to Light-Controlled Variant

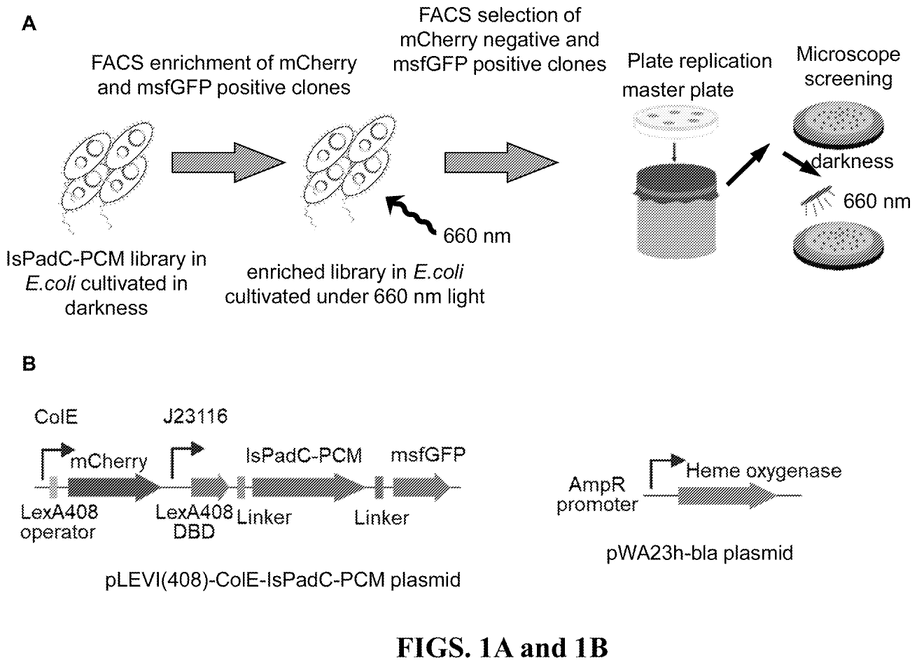

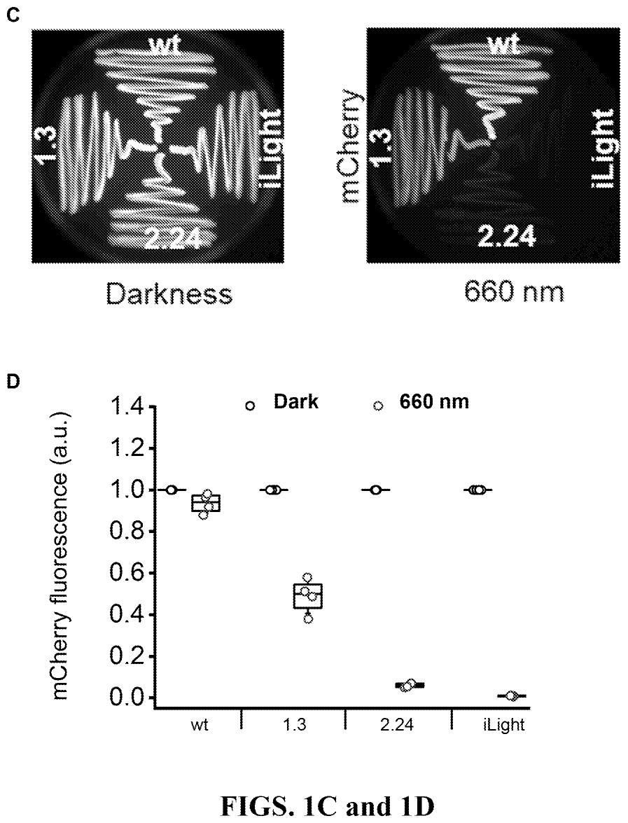

To avoid unwanted cyclase activity, the cyclase domain was removed from wild-type IsPadC, resulting in its minimal PCM module. Next, to find an IsPadC-PCM mutant able to affect the level of reporter expression in bacteria, molecular evolution was performed (FIG. 1A), in which repression of the mCherry expression (reporter) was used as a readout (FIG. 1B). To this end, a high-throughput screening approach was developed in which NIR light-induced changes of an IsPadC-PCM oligomeric state were linked through a synthetic circuit to the expression of the mCherry reporter protein. The IsPadC-PCM was fused to a C-terminus of the DNA-binding domain (DBD: amino acid residues 1-87) (Zhang, A. P., et al. Nature 466, 883-886 (2010)) of LexA408 mutated repressor of the E. coli SOS regulon that binds mutated operator and does not interfere with endogenous wild-type LexA protein and operator regions in bacterial SOS signaling pathway.

Two low-copy plasmids termed pWA23h-bla and pLEVI(408)-ColE-IsPadC-PCM, were co-transformed in TOP10 cells (FIG. 1B). The pWA23h-bla plasmid encoded heme oxygenase for BV synthesis in E. coli (Piatkevich, K. D., et al. Nat Commun 4, 2153 (2013)) under control of a constitutively active weak β-lactamase promoter. The second plasmid encoded a light-sensitive repressor LexA408DBD-IsPadC-PCM-msfGFP under control of a weak constitutive promoter J23116 and the mCherry reporter under the control of a constitutive promoter ColE with the LexA408 operator located after the promoter. Changes of the IsPadC-PCM oligomeric state after photoconversion to the Pfr state should result in the formation of a functional dimer of the LexA408 DNA binding domains. This dimer would then bind its cognate operator sequence placed after the constitutively active ColE promoter and repress the expression of mCherry.

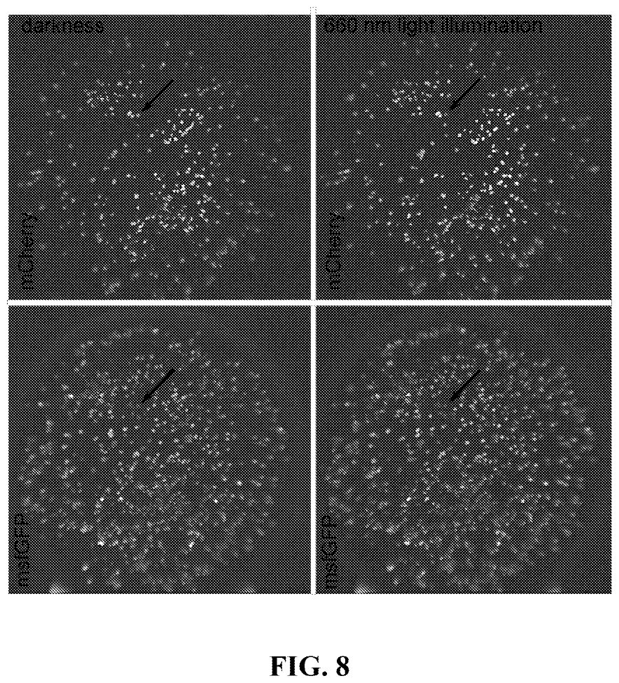

To facilitate cell sorter selection of bacterial cells with the repressed mCherry expression, the C-terminus of IsPadC-PCM was fused with a monomeric superfolder GFP (msfGFP) protein, allowing selection of the cells with full-length IsPadC-PCM. Moreover, the msfGFP signal can be used to normalize the mCherry signal during the screening of clones from the colony replicas (FIG. 8).

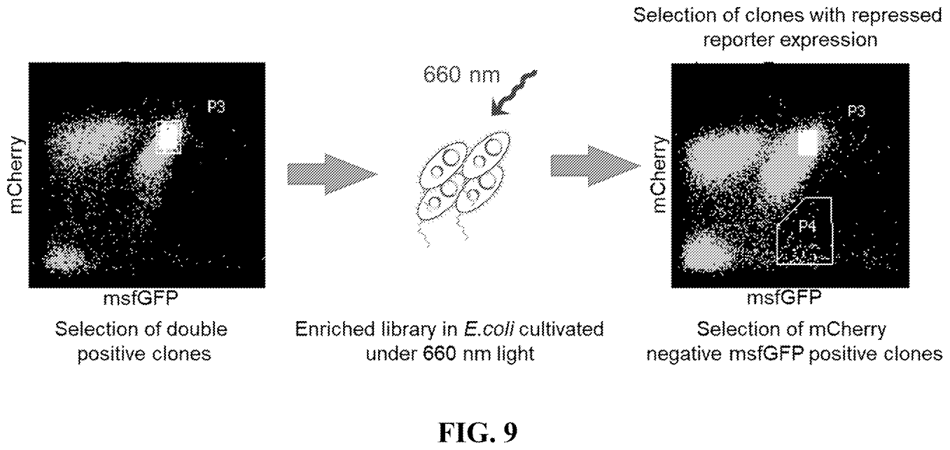

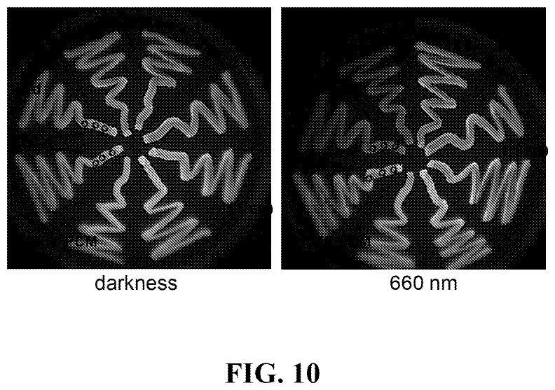

Libraries of the random IsPadC-PCM mutants in bacterial cells were grown overnight in darkness and enriched for mCherry and msfGFP positive cells using a cell sorter. The enriched library was then grown overnight under 660 nm light, and the msfGFP positive and mCherry negative cells were collected (FIG. 9). To further screen the collected cells on Petri dishes, the bacterial clones were replicated and cultivated in darkness and under 660 nm light. After the initial screening, the clones with the highest ratio of the mCherry repression were characterized in detail (FIG. 10).

As a result, after the first round of mutagenesis, a clone 1.3 with the 2-fold decrease of the mCherry signal was selected. After the next two rounds of random mutagenesis, an IsPadC-PCM variant having 9 amino acid substitutions (Table 2) was obtained, which resulted in ˜115-fold repression of the ColE-driven mCherry reporter expression (FIGS. 1C-D and FIG. 11). This variant was named iLight (Table 1).

iLight-Based Optogenetic System for Repression of Protein Production.

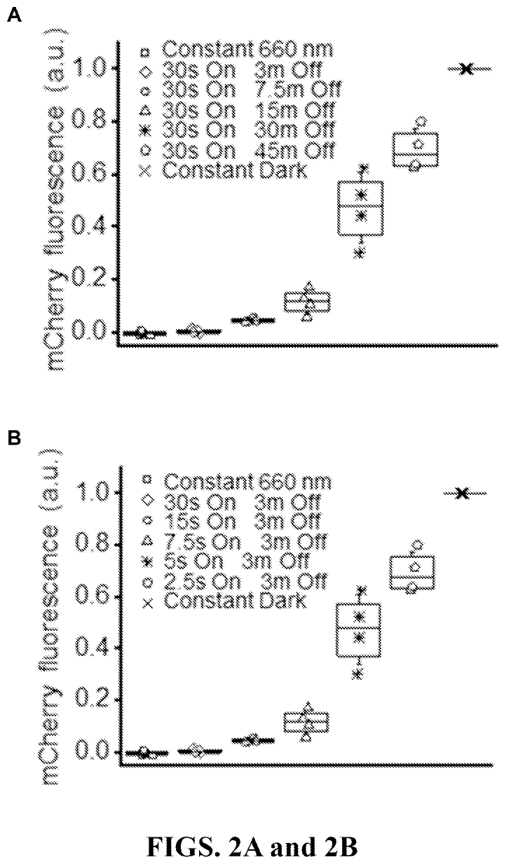

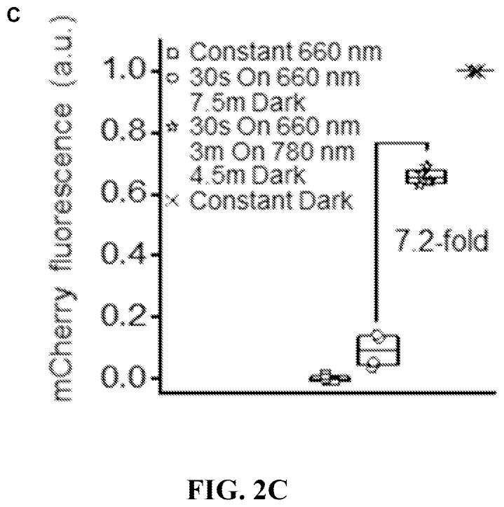

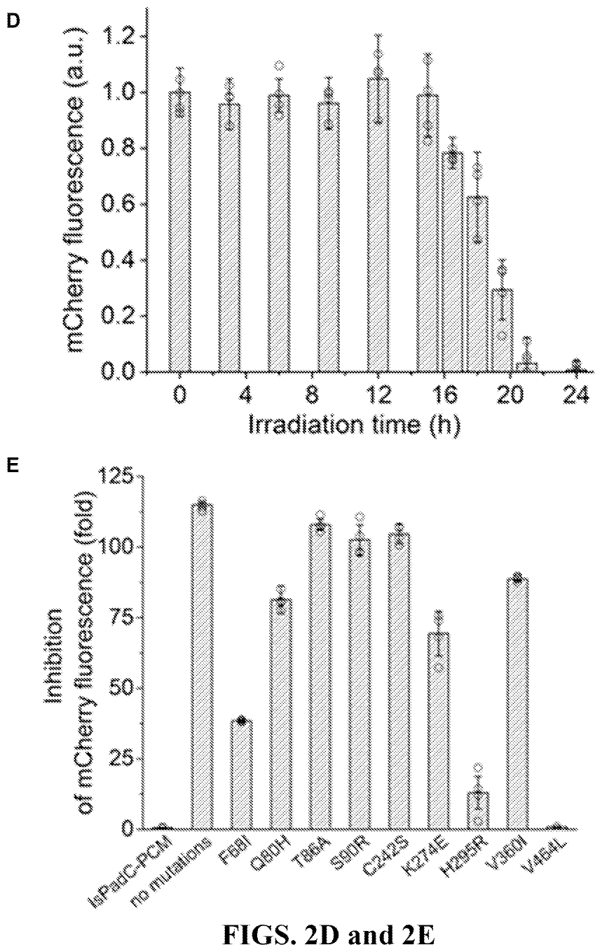

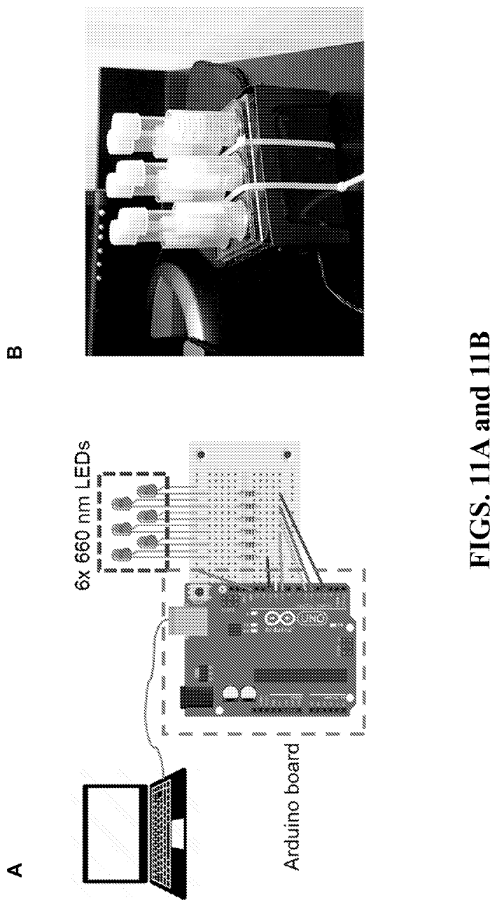

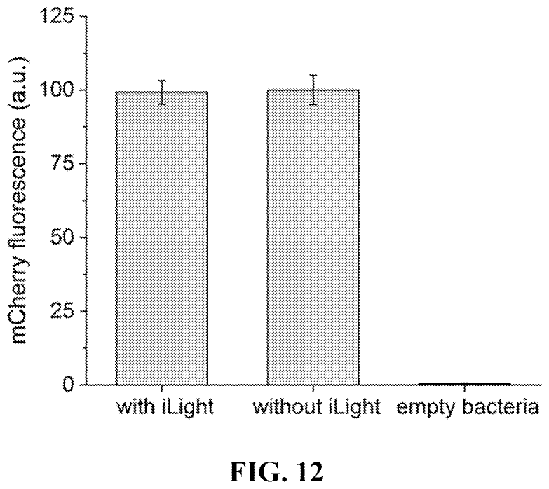

To determine the optimal illumination regime of the iLight-based repression system in bacterial cells, different illumination conditions was next studied (FIGS. 2A and 2C). A multichannel 660 nm LED array (FIG. 12) was assembled to illuminate bacteria grown in 15 ml tubes. The LED array was based on Arduino microprocessor programmed to study the dependence of the repression efficiency on the duration of Off (FIG. 2A) and On (FIG. 2B) time of 660 nm illumination. These scripts control both the On/Off illumination cycle and the light power for each tube.

The bacterial iLight optogenetic system enabled the fine-tuning of the mCherry protein repression by varying of 660 nm On/Off illumination cycle. 15 sec of 660 nm illumination sufficed to repress mCherry expression with a 115-fold contrast (FIGS. 2A and 2B). Furthermore, the repression could be switched off by iLight photoconversion back from the Pfr state to the Pr state with 780 nm light. When the illumination cycle consisted of 30 sec of 660 nm light, followed by 3 min of 780 nm light and 4.5 min in darkness, bacteria restored up to 75% of the mCherry expression (FIG. 2C). Notably, in the dark, the expression level of the mCherry reporter did not depend on whether the iLight system was co-expressed or not (FIG. 12).

Next, whether the iLight system is able to repress the gene expression when it is ongoing was tested. Bacteria were cultured for a total 24 h, with various darkness and subsequent 660 nm illumination periods. Repression of the mCherry expression was observed for the darkness periods up to ˜8 h, which were followed by the iLight activation (FIG. 2D). The mCherry repression reached 50% for 5.5 h of ongoing expression followed by 18.5 h of the 660 nm illumination.

To determine the contribution of each of nine amino acid substitutions found in iLight (Table 2) on its gene suppression activity, they were sequentially reverted to those in wild-type IsPadC-PCM to determine the efficiency of the resulting single-point iLight mutants on the repression of the gene expression (FIG. 2E). Each of the Q80H, T86A, S90R, C242S, K274R, and V360I single-point mutations had a minor effect on the inhibition performance of the bacterial iLight system. In contrast, the F68I, H295R, and V464L amino acid substitutions significantly affected the iLight performance, with the V464L substitution lowering the inhibition efficiency of the gene expression to only 1.6-fold.

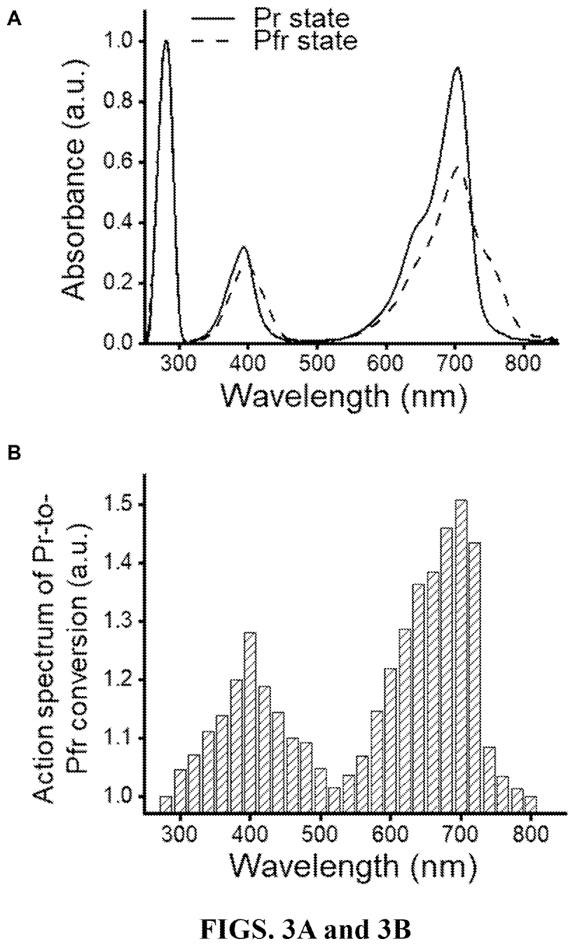

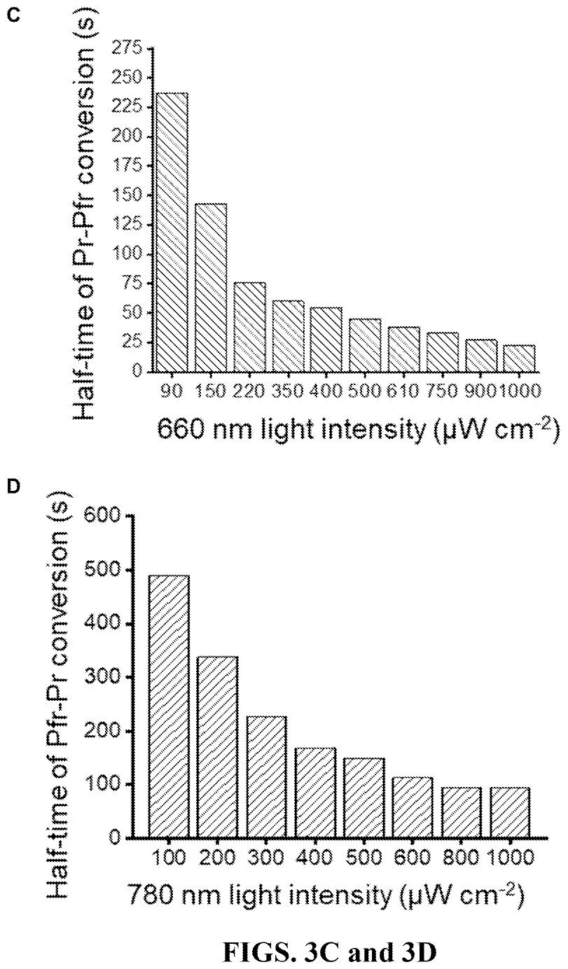

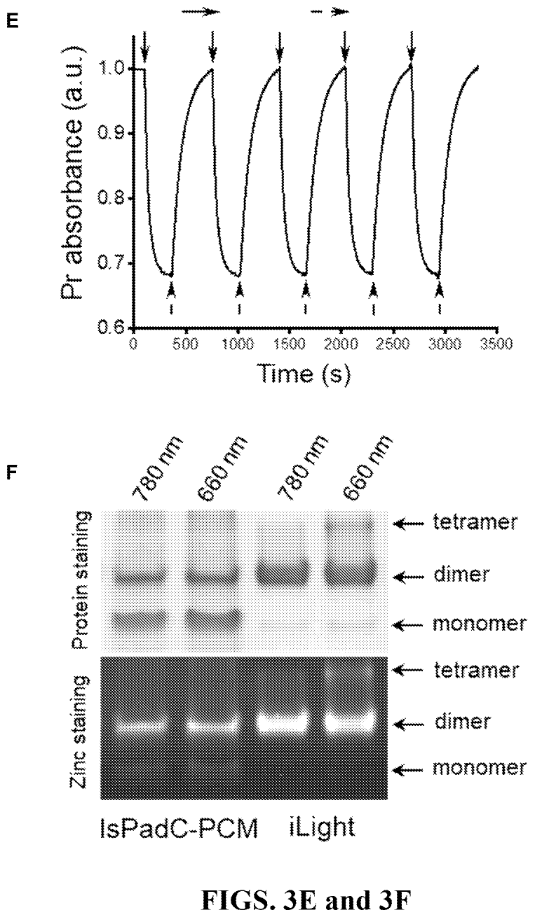

Characterization of the Purified iLight Variant. iLight originates from canonical IsPadC BphP that adopts Pr form as a ground state (Gourinchas, G. et al. Sci Adv 3, e1602498 (2017)). In its ground state, the iLight variant absorbed at 394 nm (Soret band) and 704 nm (Q band) (FIG. 3A). Upon 660 nm illumination, it photoconverted into the Pfr state with absorption maxima at 396 nm Soret band and notable shoulder at 752 nm corresponding to Q band. A 1.6-fold decrease in absorbance at 704 nm in the Pfr state was observed, similarly to wild-type IsPadC. The Pr→Pfr photoconversion can be achieved by ˜640-720 nm light, with the maximum photoconversion efficiency at 700 nm (FIG. 3B). The half-time of the Pr→Pfr photoconversion (half of the protein was converted) was 23 s at 1000 μW cm⁻² and increased to 237 s at 90 μW cm⁻² (FIG. 3C).

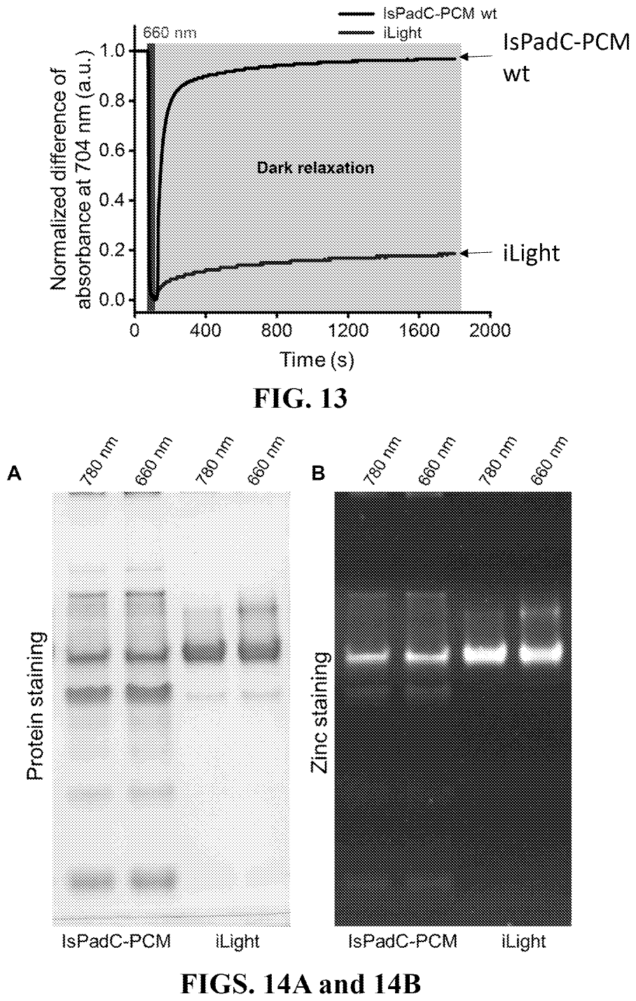

iLight returns from the Pfr state back to the ground Pr state after dark relaxation or after illumination with 780 nm light. The kinetics of Pfr→Pr dark relaxation was substantially slower than with 780 nm light (FIG. 14). Moreover, the iLight dark relaxation was substantially slower than that of wild-type IsPadC-PCM (FIG. 13). The half-time of the Pfr→Pr photoconversion of iLight depended on the 780 nm light intensity, ranging from 91 s at 1000 μW cm⁻² to 490 s at 100 μW cm⁻² (FIG. 3D). Multiple cycles of the reversible Pr-Pfr photoswitching did not lead to notable changes in the iLight absorbance at 704 nm (FIG. 3E).

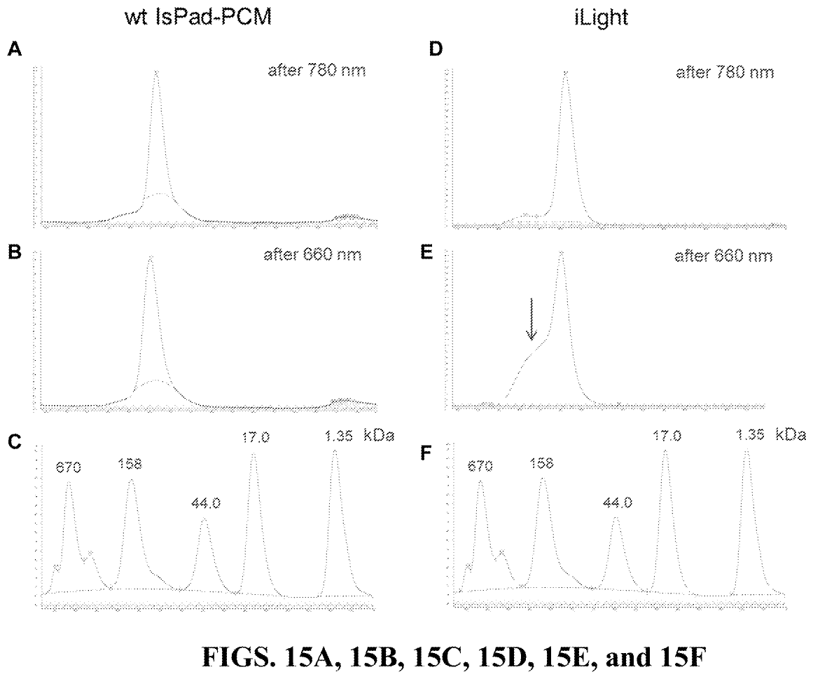

Native PAGE followed by Zn²⁺ staining for biliverdin chromophore (FIG. 3F and FIG. 14 with full gel) and size-exclusion chromatography (FIG. 15) indicated that in the Pr state iLight exists as a dimer. Its photoconversion to the Pfr state with 660 nm light causes the formation of a tetrameric fraction. In contrast, wild-type IsPadC-PCM did not form a notable tetrameric fraction after 660 nm illumination. These data indicate a dimer-to-tetramer formation as the mechanism of action of the iLight-based optogenetic constructs.

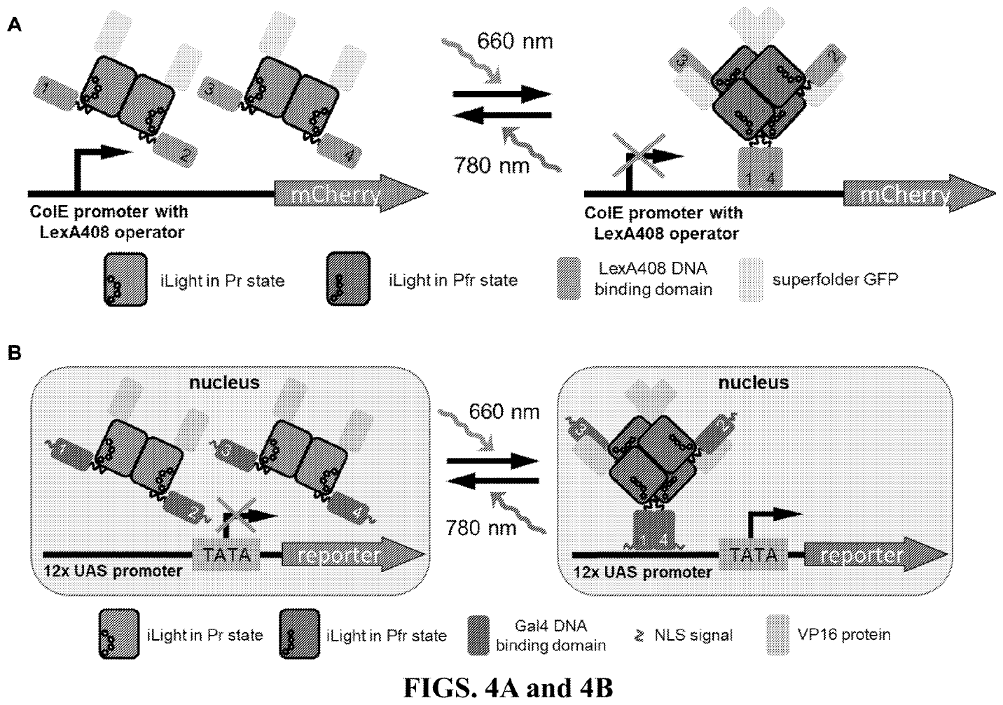

It has been shown that similarly to other canonical BphPs, wild-type IsPadC and IsPadC-PCM form the tight parallel dimers ((Gourinchas, G. et al. Sci Adv 3, e1602498 (2017)); Gourinchas, G., et al. Elife 7 (2018)). However, unlike other BphPs in which N-termini extended from the PAS domain are typically unstructured, an N-terminus of IsPadC in the Pr ground state forms an α-helical structure. Moreover, it is turned by its N-terminus towards the PHY domain because of the interaction with the tongue structure of the PHY domain. In the Pr state, the PHY-tongue forms two anti-parallel β-sheets (Takala, H. et al. Nature 509, 245-248 (2014)). Moreover, unlike other BphPs, 660 nm light causes complete Pr→Pfr transition and the PHY-tongue restructuring into an α-helix in only one protomer of the IsPadC-PCM dimer. Another IsPadC-PCM protomer in the photoactivated dimer still remains in the Pr state. However, the α-helix of the PHY-tongue in the photoactivated Pfr-state protomer is unable to interact and, consequently, stabilize the N-terminal helical structure, causing its partial unfolding and turning by almost 180 degrees away from the PHY-tongue. These structural features of IsPadC-PCM do not allow to bring close two DNA-binding domains fused to the N-terminus of each protomer in the photoactivated IsPadC-PCM dimer. To achieve that, two dimers should be assembled and then photoactivated, providing a possibility for two DNA-binding domains, one from each dimer, to form an active transcription factor dimer at the DNA sequence. This proposed mechanism of action is implemented in the iLight optogenetic system (FIG. 4A).

iLight-Induced Transcription Activation in Mammalian Cells.

It has been shown that a Gal4-VP16 fusion protein efficiently activates gene transcription in mammalian cells by binding to repeats of yeast-derived upstream activation sequence (UAS) (Sadowski, I., et al. Nature 335, 563-564 (1988)). The Gal4-UAS gene transcription system is widely used in model organisms, including insects, fishes, and mammals (Mallo, M. et al. Front Biosci 11, 313-327 (2006)).

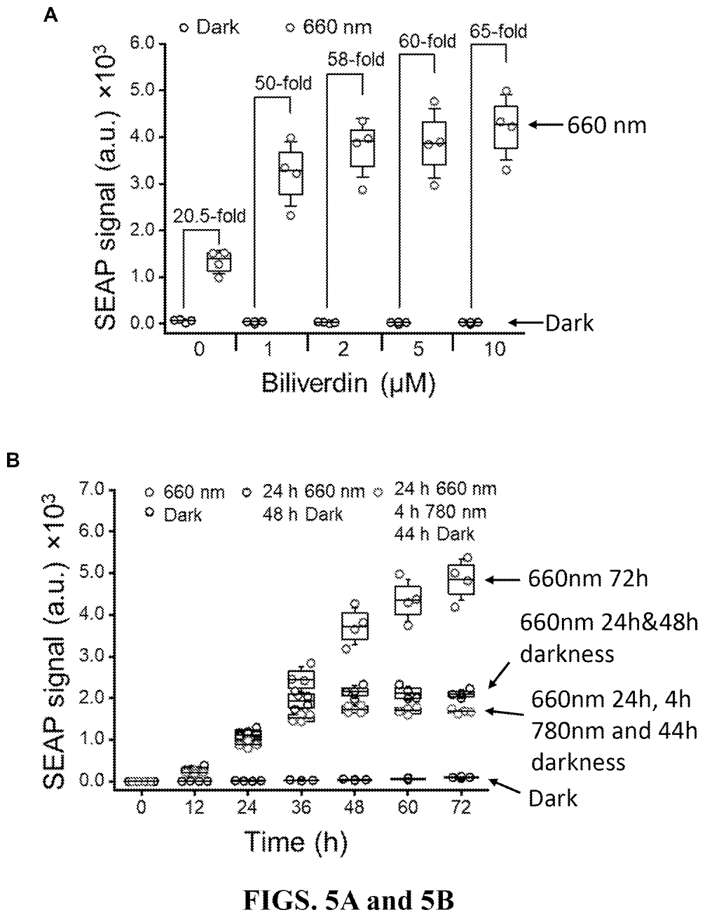

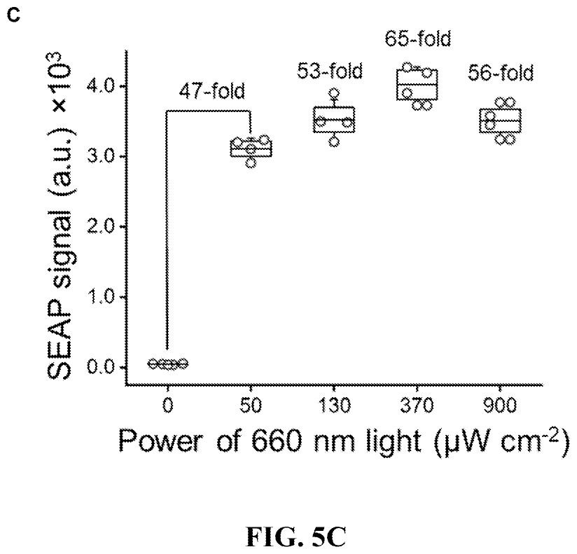

To develop a light-inducible gene transcription system with iLight in mammalian cells, a DNA-binding domain (DBD: N-terminal 1-65 amino acid residues) of the yeast activator Gal4 (Gal4-DBD) was fused to the N-terminus of codon-optimized iLight, and VP16 was fused to the C-terminus of iLight (FIG. 4B and Table 1). HeLa cells stably expressing NLS-Gal4-DBD-iLight-VP16 were transfected with pG12-SEAP reporter plasmid containing 12×UAS repeats upstream of secreted alkaline phosphatase (SEAP) gene. 660 nm illumination for 48 h increased the SEAP production by ˜20.5-fold as compared to the cells in darkness without the supply of exogenous BV and by ˜65-70-fold in cells supplemented with 10 μM of BV (FIG. 5A). The time-course of SEAP production revealed ˜19-fold SEAP increase after 24 h and up to ˜70-fold after 72 h of 660 nm illumination as compared to the dark-treated cells (FIG. 5B).

Next, how fast the light-induced transcriptional activation could be terminated was studied. Cells were illuminated with 660 nm light for 24 h and then kept in darkness. The SEAP reporter production increased ˜1.7-fold during the first 12 h in darkness, due to pre-accumulation of SEAP's mRNA, and then the SEAP level stabilized (FIG. 5B). Similar SEAP kinetics was observed for cells illuminated with 780 nm light for 4 h right after the 24 h illumination period with 660 nm light. About 1.7-fold increase of SEAP reporter production during the first 12 h after 660 nm illumination without any following increase was observed (FIG. 5B).

The dependence of the SEAP expression on the 660 nm light intensity was further tested (FIG. 5C). After an initial increase of the SEAP production in the 0-50 μW cm⁻² light intensity range, the SEAP level reached a plateau in the 50-900 μW cm⁻² range.

Characterization of the iLight Optogenetic System in Primary Neurons.

To characterize the system in neurons, an AAV vector expressing iLight system and the reporter AAV vectors expressing mCherry fluorescent protein and CheRiff channelrhodopsin were constructed. In all vectors, the gene expression was driven by the calcium/calmodulin-dependent kinase II (CaMKII) promoter commonly used to express proteins specifically in cortical and hippocampal excitatory neurons. The neurons were isolated from hippocampi of newborn mice, cultured on glass coverslips, and transduced on a day in vitro 7 (DIV7) with iLight system and reporter AAVs. After the co-transduction, the neurons were kept in darkness with 2 μM of BV. On DIV12, the cells were illuminated with 660 nm light (500 μW cm⁻², 30 s On, 180 s Off) to induce reporter expression. The illumination continued for 5 days, and the cells were imaged afterward.

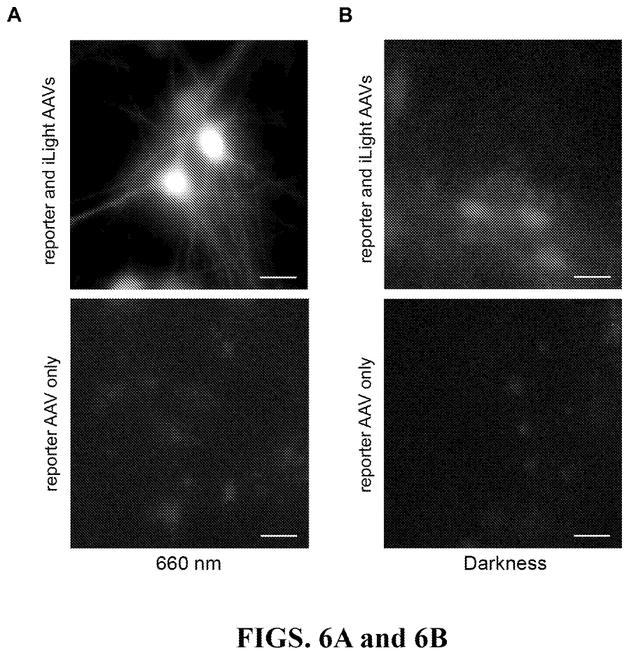

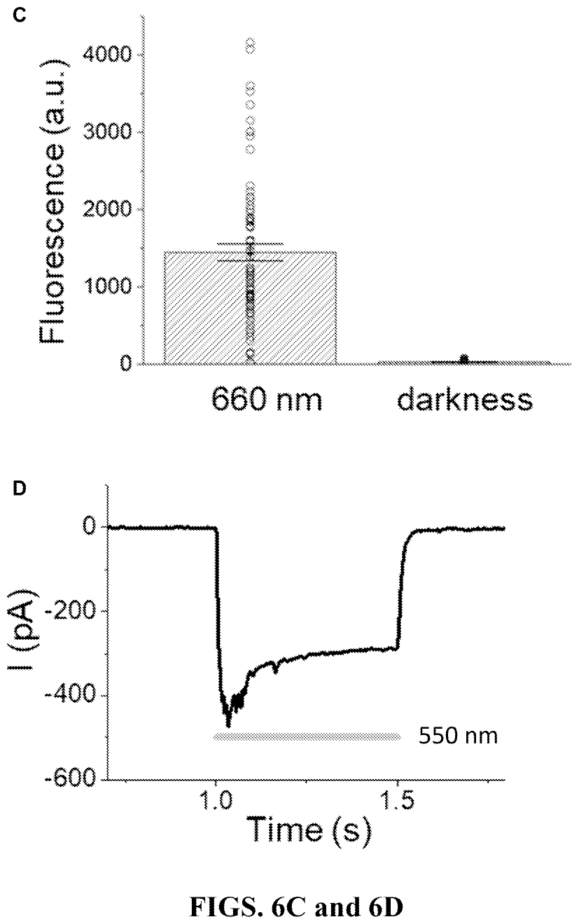

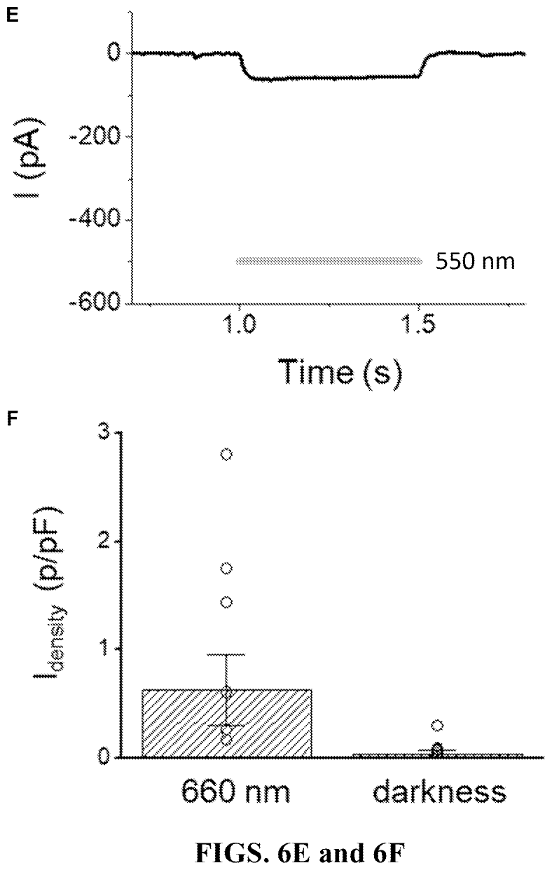

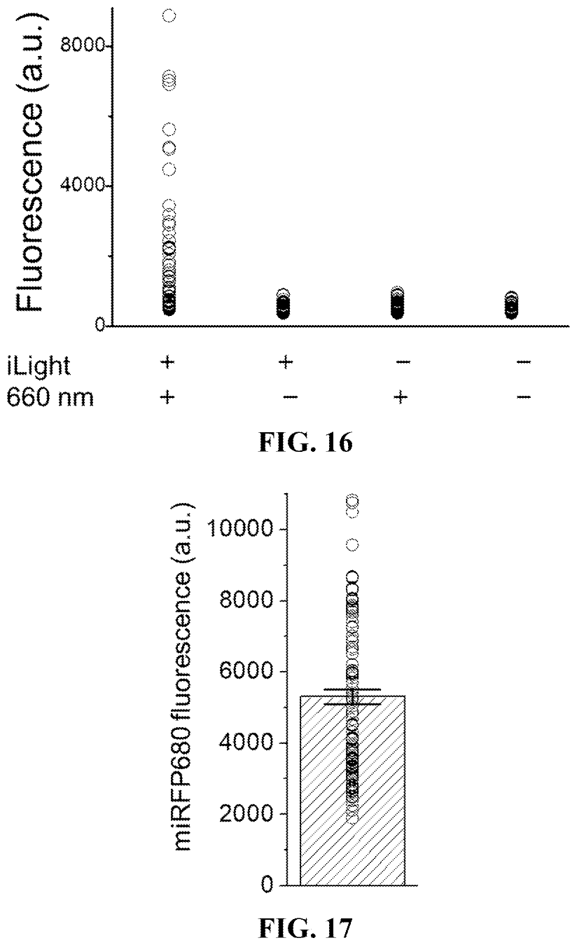

Bright fluorescence of the mCherry reporter was observed in neurons illuminated with 660 nm light (FIG. 6A), whereas in neurons kept in darkness the fluorescence intensity was substantially lower (FIG. 6B). The mCherry fluorescence levels varied substantially between individual cells exposed to 660 nm light (FIG. 16). Similar high variability was observed in experiments in which neurons were transduced with AAV encoding another fluorescent protein driven by constitutive CaMKII promoter (FIG. 17). After subtraction of the fluorescence levels in the control neuronal cultures transduced with mCherry reporter alone, the light-induced mCherry expression was significantly higher than in darkness (1446±956.7 arbitrary units under light, 28.9±23.9 arbitrary units in darkness, T=6, df=113, p=2.3×10⁻⁸) (FIG. 6C). The results showed that the iLight optogenetic system can induce protein expression in primary cultured cells, such as hippocampal neurons.

Multiplexing of the iLight Optogenetic System with Channelrhodopsin.

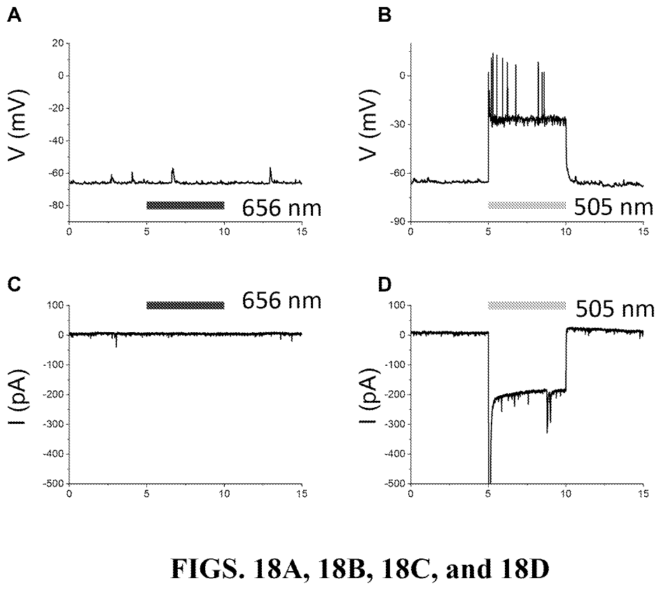

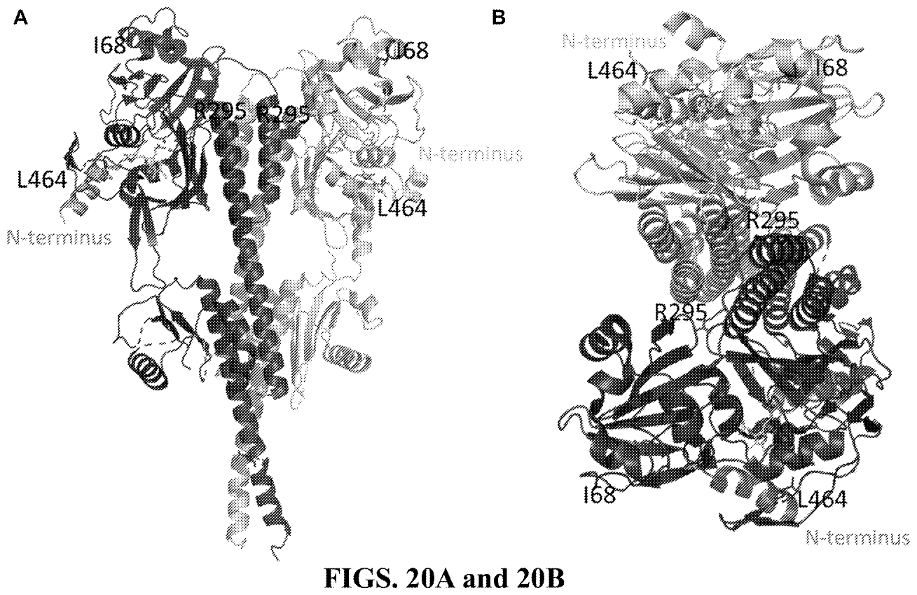

The absorption spectrum of iLight does not overlap with the activation spectrum of CheRiff channelrhodopsin, which is peaked at −460 nm (FIG. 20). Consequently, in neurons co-transduced with the iLight system and CheRiff reporter AAVs, the 660 nm illumination did not cause photocurrents and did not depolarize the cells (FIG. 18). Whether the expression of CheRiff in neurons can be controlled with iLight system in the same way as the expression of mCherry was tested. The neurons co-transduced with iLight and CheRiff were illuminated with 660 nm light (500 μW cm²) starting on DIV12 (control culture was kept in darkness), and photocurrents induced by 505 nm light were measured on DIV17.

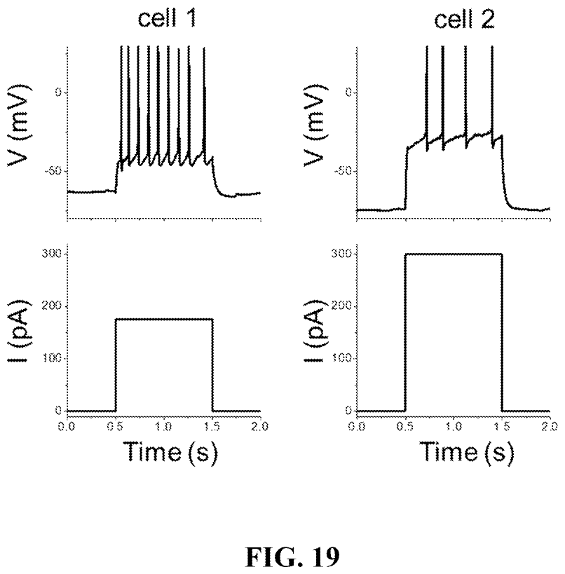

It was observed that all patch-clamped neurons fired action potentials when the current (150-300 pA) was injected through the patch electrode (FIG. 19). For photocurrent measurements, the neurons were held at −70 mV in voltage clamp mode. The 1 s flashes of 505 nm light of 200 mW cm^{a} activated CheRiff and induced photocurrents in all transduced neurons (example cell current traces are shown in FIGS. 6D and 6E). The resulting steady-state photocurrent values were divided by the values of cell membrane capacitance to obtain a current density, which is an estimate of the number of functional channelrhodopsin molecules per unit of the cell surface. The photocurrent density reached 2-3 pA/pF in some cells. Average photocurrent density in neurons kept under light was significantly higher than in cells kept in darkness (0.82±0.33 and 0.11±0.03 pA/μF respectively, 10 cells in each group, T=2.17, df=18, p=0.044). After subtraction of average photocurrent density values in cells transduced with CheRiff alone (0.19±0.06 under light, 0.08±0.03 pA/μF in darkness, 5 cells in each group), the average photocurrent density increase due to iLight-mediated activation was 20.1-fold higher in neurons incubated under 660 nm light than in the cells kept in darkness (FIG. 6F). These experiments validated that the iLight optogenetic system can drive expression of channelrhodopsin actuator in neurons, enabling crosstalk-free spectral multiplexing with optogenetic tools activated by blue-green light. iLight-driven light-activation of protein expression in vivo.

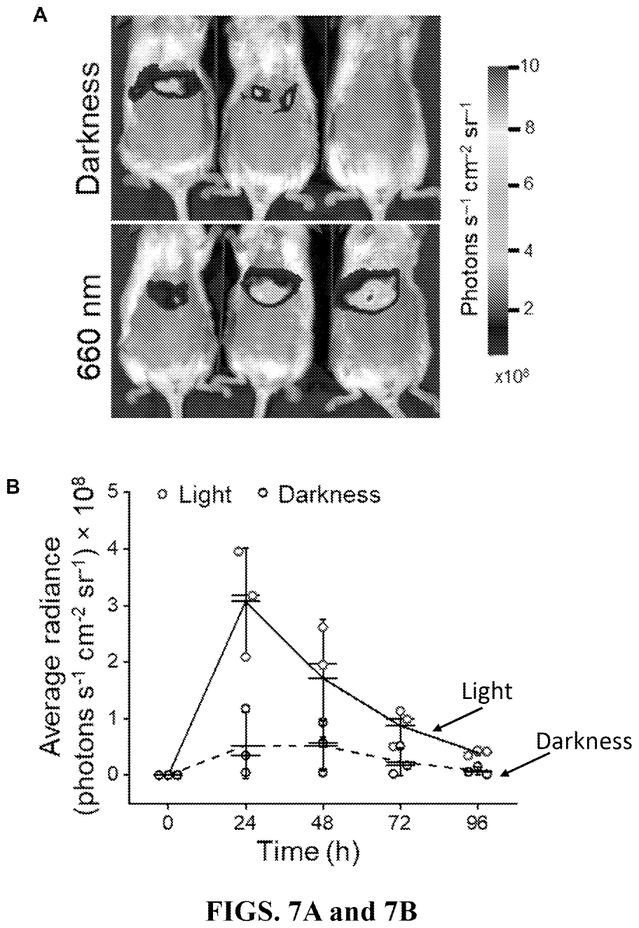

For activation of gene transcription activation in deep tissues, the kinetics of light-induced Renilla renformis luciferase (RLuc8) expression in livers of mice, which were hydrodynamically co-transfected with the plasmid encoding NLS-Gal4-DBD-iLight-VP16 construct and the pG12-RLuc8 reporter plasmid, was tested (FIG. 7A). The maximum of the RLuc8 signal in the livers was observed after 24 h of 660 nm illumination. The RLuc8 signal was ˜6-fold higher in the illuminated mice than in the mice kept in darkness (FIG. 7B). The difference of the RLuc8 expression between the illuminated and dark-treated animals was observed up to 96 h after the hydrodynamic transfection. These results showed that the iLight optogenetic system performed well in mouse tissues in vivo, and achieved the maximum contrast twice faster (24 h) than the heterodimerization-based RpBphP1-RpPpsR2 two-component system (48 h).

### DISCUSSION

Existing NIR optogenetic systems consist of several protein components of large size and multidomain structure, resulting in low efficiency and high background. This disclosure provides single-component NIR systems consisting of an evolved photosensory core module of Idiomarina sp. bacterial phytochrome, named iLight, which are smaller and packable in an adeno-associated virus (AAV). As shown above, iLight was characterized in vitro and in gene transcription repression in bacterial and gene transcription activation in mammalian cells. Bacterial iLight system shows 115-fold repression of protein production. Comparing to multi-component NIR systems, the mammalian iLight system exhibits higher activation of 65-fold in cells and faster 6-fold activation in deep tissues of mice. Neurons transduced with viral-encoded iLight system exhibit 50-fold induction of fluorescent reporter. NIR light-induced neuronal expression of green-light-activatable CheRiff channelrhodopsin causes 20-fold increase of photocurrent and demonstrates efficient spectral multiplexing.

To engineer the light-controlled iLight variant of IsPadC-PCM the directed molecular evolution approach was developed, in which light-induced change of oligomeric state of the IsPadC-PCM mutants resulted in the dimerization of LexA408-DBD domains and consequent repression of the reporter protein production. Notably, in this approach, the DNA binding domain of LexA408 mutated repressor and its operator are orthologous in E. coli cells and do not affect endogenous processes.

Structural and biochemical (FIG. 3F and FIG. 15) analyses favor the tetramerization mechanism of action of the iLight system, with the I68F, R259H, and L464V amino acid substitutions being the most important for the system performance (FIG. 2E). Based on the crystal structure of the IsPadC (PDB ID: 6ET7), the Ile68 residue is located in the long, bent, uninterrupted α-helix of the PAS domain (FIG. 20). According to the HDX-MS analysis, the PAS domain of IsPadC does not directly contribute to the Pr→Pfr transition, indicating that I68F substitution adds to the overall iLight rigidity and may facilitate the biliverdin incorporation. The Arg295 residue is located at the GAF-proximal terminal part of the GAF-PHY α-helix and positioned in proximity to the α-helix of the GAF domain of another protomer in the IsPadC dimer. R295H mutation may be involved in the light-induced interaction of the two iLight dimers, resulting in their tetramerization.

The Leu464 residue is located in the conserved in BphPs⁴⁶⁴LXPRXSF⁴⁷⁰ amino acid motif of the PHY-tongue, which is involved in the stabilization of the Pr and Pfr states. Leu464 accelerates the docking of Arg467 with Asp207 and Tyr263 surrounding the biliverdin chromophore during the Pfr→Pr transition. In addition, iLight exhibits a significantly reduced relaxation rate in darkness as compared to wild-type IsPadC-PCM (FIG. 13). iLight benefits from the slow Pfr→Pr dark-reversion because the dissociation of the iLight tetramers is delayed, which results in longer association of the DNA-binding domains and, consequently, their interaction with the corresponding DNA sequence. This is consistent with the fact that the mutation at position 464 has appeared early in the molecular evolution of iLight (Table 2).

It was hypothesized that the other six amino acid substitutions observed in iLight (Table 2) improved the protein folding and the BV binding, which is needed for the formation of the IsPadC-PCM dimer (FIG. 3F).

The mammalian iLight optogenetic system was successfully applied to light-activated expression of the reporter proteins under CMV promoter in conventional HeLa cells and under neuron-specific CaMKII promoter in primary neurons. Experiments in HeLa cells provided up to 65-70-fold increase in the production of the SEAP reporter but had limited reversibility (FIG. 5B). In contrast, the multiple cycles of photoswitching between the Pr and Pfr states were observed with the purified iLight protein (FIG. 3). The apparent irreversibility in the mammalian cells was caused by several factors, including high stability of the SEAP's mRNA and high-affinity interaction of the dimerized via tetrameric iLight Gal4 DBDs with the UAS repeats.

In neurons, because CheRiff channelrhodopsin is activated by blue-green light (peak of activation at ˜475 nm), 660 nm illumination used to induce iLight-mediated gene transcription did not affect CheRiff activity (FIG. 18). The large spectral separation allowed combining channelrhodopsin actuator and iLight system in the same cells, resulting in efficient spectral multiplexing. Since the CheRiff is not activated by a red light while being produced, this combination preserves natural neuronal activity until a sufficient amount of CheRiff molecules is expressed. This approach is more suitable than combining shorter wavelength light (e.g., 470 nm) for activation of gene expression and red light for activation of red-shifted channelrhodopsins, because channelrhodopsins sensitive to red light, such as VChR1, still retain responsivity to shorter wavelengths. More generally, the spectral multiplexing enabled by the iLight system could be used for co-expression of other molecular tools activated or excited by blue-green light, including LOV- and CRY2-based optogenetic tools (Shcherbakova, D. M., et al. Annu Rev Biochem 84, 519-550 (2015); Leopold, A. V., et al. Chem Soc Rev 47, 2454-2484 (2018)) and biosensors based on GFP-derived fluorescent proteins (Shcherbakova, D. M., et al. Angew Chem Int Ed Engl 51, 10724-10738 (2012)).

The photocurrents generated by CheRiff with short flashes of 505 nm light were sufficient to depolarize neurons and drive action potentials. The magnitude of the resulting current densities was comparable to that observed in CheRiff-expressing neurons in other studies (e.g., 2.8-4.4 pA/μF in the culture of dorsal root ganglion cells) (Lou, S. et al. The Journal of neuroscience: the official journal of the Society for Neuroscience 36, 11059-11073 (2016)). Channelrhodopsins, including CheRiff are widely used to simulate spiking in neurons in the brains of various animals.

The results indicate that the iLight optogenetic system can be further applied to control neuronal activity in vivo. It was hypothesized that the light-induced increase of the CheRiff-medicated photocurrent in neurons in the mouse brain could substantially enhance their firing.

The observed substantial increase of the RLuc8 reporter expression in the liver of mice (FIG. 7) indicates that the iLight optogenetic system can be used in deep tissue applications in vivo. NIR light that triggers iLight exhibits deeper tissue penetration, lower scattering, and lower phototoxicity than light in the visible range.

To apply both types of the optogenetic systems in vivo, three AAVs are required. In contrast, the iLight one-component system requires only two AAVs, as it was shown in neurons (FIG. 6), thus, substantially simplifying its applications in vivo.

In addition, because of the smaller size of iLight (60 kDa) than two-component NIR systems (full-length dimeric phytochrome of 80 kDa and dimeric interacting partner of 50-60 kDa), the iLight-based construct is synthesized faster and, correspondingly, provides the maximal activation contrast (reporter expression level) twice faster (FIG. 7) than the two-component systems (Kaberniuk, A. A., et al. Nat Methods 13, 591-597 (2016); Redchuk, T. A., et al. Nat Chem Biol 13, 633-639 (2017)).

Almost twice larger activation contrast achieved by the iLight system in mammalian cells (65-70-fold) than that by the RpBphP1-RpPpsR2 and RpBphP1-QPAS1 systems under the similar conditions (35-40-fold) may result from the lower background activation in the darkness of iLight than RpBphP1.

1. A polynucleotide comprising a nucleotide sequence encoding a chimeric polypeptide comprising a light-responsive polypeptide linked to a DNA binding domain, wherein the light-responsive polypeptide comprises an amino acid sequence having at least 90% sequence identity to SEQ ID NO: 1 or comprises the amino acid sequence of SEQ ID NO: 1.

2. The polynucleotide of claim 1, wherein the light-responsive polypeptide when associated with a chromophore is capable of switching from a first state to a second state when exposed to illumination by a first wavelength and switching from the second state to the first state when exposed to illumination by a second wavelength, or returning from the second state to the first state in darkness.

3. The polynucleotide of claim 1, wherein the light-responsive polypeptide is a variant of Idiomarina sp. A28L Phytochrome activated diguanylyl Cyclase (IsPadC).

4. The polynucleotide of claim 1, wherein the light-responsive polypeptide comprises an N-terminal photosensory core module (PCM) of IsPadC.

5. The polynucleotide of claim 1, wherein the light-responsive polypeptide comprises at least one mutation at position I68, H80, A86, R90, S242, R274, R295, I360, or L464.

6. The polynucleotide of claim 5, wherein the at least one mutation comprises one or more substitutions selected from the group consisting of I68F, H80Q, A86T, R90S, S242C, R274K, R295H, I360V, L464V, and combinations thereof.

7. The polynucleotide of claim 6, wherein the at least one mutation comprises at least one of F68I, H295R, and V464L substitutions.

8. The polynucleotide of claim 6, wherein the at least one mutation comprises the F68I, H295R, and V464L substitutions.

9. The polynucleotide am of claim 1, wherein the light-responsive polypeptide comprises an amino acid sequence having at least 90% sequence identity to SEQ ID NO: 2 or comprises the amino acid sequence of SEQ ID NO: 2.

10. The polynucleotide of claim 1, wherein the light-responsive polypeptide is linked to the DNA binding domain via a peptide linker.

11. The polynucleotide of claim 1, wherein the nucleotide sequence is operably linked to a promoter.

12. The polynucleotide of claim 2, wherein the chromophore is a biliverdin chromophore.

13. The polynucleotide of claim 2, wherein the first state is a Pr state and the second state is a Pfr state.

14. The polynucleotide of claim 2, wherein at least a portion of the light-responsive polypeptide exists in a dimeric form in the first state.

15. The polynucleotide of claim 2, wherein at least a portion of the light-responsive polypeptide exists in a tetrameric form in the second state.

16. The polynucleotide of claim 15, wherein at least a pair of the DNA binding domains of the tetrameric form of the light-responsive polypeptide are capable of binding to a DNA recognition site.

17. The polynucleotide of claim 14, wherein a PHY-tongue of only one protomer of the dimeric form of the light-responsive polypeptide that is constituted by two anti-parallel β-sheets in the first state is restructured to an α-helix in the second state when exposed to illumination by the first wavelength.

18. The polynucleotide of claim 2, wherein the first wavelength and the second wavelength are in far-red or near-infrared spectrum.

19. The polynucleotide of claim 2, wherein the first wavelength is between about 600 nm and about 680 nm.

20. The polynucleotide of claim 19, wherein the first wavelength is about 660 nm.

21. The polynucleotide of claim 2, wherein the second wavelength is between about 740 nm and about 800 nm.

22. The polynucleotide of claim 21, wherein the second wavelength is about 780 nm.

23. The polynucleotide of claim 1, wherein the DNA binding domain comprises a DNA binding motif.

24. The polynucleotide of claim 23, wherein the DNA binding motif comprises a helix-turn-helix, a homeodomain, a leucine zipper, a helix-loop-helix, or a zinc finger.

25. The polynucleotide of claim 23, wherein the DNA binding domain comprises a Gal4 DNA binding domain, a Lex-A DNA binding domain, an NF-κB DNA binding domain, a cro repressor DNA binding domain, a Lac repressor DNA binding domain, a GCN4 DNA binding domain, an Opaque-2 DNA binding domain, or a TGAIa DNA binding domain.

26. The polynucleotide of claim 1, further comprising a second nucleotide sequence encoding a second light-responsive polypeptide.

27. The polynucleotide of claim 26, wherein the second light-responsive polypeptide comprises a rhodopsin.

28. A light-responsive polypeptide comprising an amino acid sequence having at least 90% sequence identity to SEQ ID NO: 2, 3 or 4 or comprising the amino acid sequence of SEQ ID NO: 2, 3 or 4.

29. The light-responsive polypeptide of claim 28, wherein the light-responsive polypeptide is associated with a chromophore and capable of switching from a first state to a second state when exposed to illumination by a first wavelength and switching from the second state to the first state when exposed to illumination by a second wavelength, or returning from the second state to the first state in darkness.

30. The light-responsive polypeptide of claim 28, wherein the light-responsive polypeptide is a variant of a PCM of an IsPadC.

31. The light-responsive polypeptide of claim 28, comprising at least one mutation at position I68, H80, A86, R90, S242, R274, R295, I360, or L464.

32. The light-responsive polypeptide of claim 31, wherein the at least one mutation comprises one or more substitutions selected from the group consisting of I68F, H80Q, A86T, R90S, S242C, R274K, R295H, I360V, L464V, and combinations thereof.

33. The light-responsive polypeptide of claim 32, wherein the at least one mutation comprises at least one of F68I, H295R, V464L substitutions.

34. The light-responsive polypeptide of claim 32, wherein the at least one mutation comprises the F68I, H295R, and V464L substitutions.

35. The light-responsive polypeptide of claim 28, further comprising a DNA binding domain linked to the amino acid sequence via a linker.

36. The light-responsive polypeptide of claim 29, wherein the chromophore is a biliverdin chromophore.

37. The light-responsive polypeptide of claim 29, wherein the first state is a Pr state and the second state is a Pfr state.

38. The light-responsive polypeptide of claim 29, wherein at least a portion of the light-responsive polypeptide exists in a dimeric form in the first state.

39. The light-responsive polypeptide of claim 29, wherein at least a portion of the light-responsive polypeptide exists in a tetrameric form in the second state.

40. The light-responsive polypeptide of claim 39, wherein at least a pair of the DNA binding domains of the tetrameric form of the light-responsive polypeptide are capable of binding to a DNA recognition site.

41. The light-responsive polypeptide of claim 29, wherein the PHY-tongue of only one protomer of the dimeric form of the light-responsive polypeptide that is constituted by two anti-parallel β-sheets in the first state is restructured to an α-helix in the second state when exposed to illumination by the first wavelength.

42. The light-responsive polypeptide of claim 29, wherein the first wavelength and the second wavelength are in far-red or near-infrared spectrum.

43. The light-responsive polypeptide of claim 29, wherein the first wavelength is between about 600 nm and about 680 nm.

44. The light-responsive polypeptide of claim 43, wherein the first wavelength is about 660 nm.

45. The light-responsive polypeptide of claim 29, wherein the second wavelength is between about 740 nm and about 800 nm.

46. The light-responsive polypeptide of claim 45, wherein the second wavelength is about 780 nm.

47. The light-responsive polypeptide of claim 35, wherein the DNA binding domain comprises a DNA binding motif.

48. The light-responsive polypeptide of claim 47, wherein the DNA binding motif comprises a helix-turn-helix, a homeodomain, a leucine zipper, a helix-loop-helix, or a zinc finger.

49. The light-responsive polypeptide of claim 48, wherein the DNA binding domain comprises a Gal4 DNA binding domain, a Lex-A DNA binding domain, an NF-κB DNA binding domain, a cro repressor DNA binding domain, a Lac repressor DNA binding domain, a GCN4 DNA binding domain, an Opaque-2 DNA binding domain, or a TGAIa DNA binding domain.

50. A vector comprising the polynucleotide of claim 1.

51. A host cell comprising the polynucleotide of claim 1.

52. A polypeptide encoded by the polynucleotide of claim 1.

53. A composition comprising the polynucleotide of claim 1.

54. A system for modulating an expression level of a gene, comprising the polynucleotide of any one of claim 1, wherein the DNA binding domain is capable of binding to a regulatory element of the gene.

55. The system of claim 54, wherein the regulatory element is a promoter or an operator.

56. A method for modulating a gene expression level, comprising:
introducing to a cell the polynucleotide of claim 1; and
exposing the cell to illumination by a first wavelength and optionally exposing the cell to illumination by a second wavelength,
wherein the DNA binding domain is capable of binding to a regulatory element of the gene.

57. A method for modulating a gene expression level, comprising providing the polypeptide of claim 1; and
exposing the polypeptide or the host cell to illumination by a first wavelength and optionally exposing the cell to illumination by a second wavelength,
wherein the DNA binding domain is capable of binding to a regulatory element of the gene.

58. The method of claim 56, wherein the regulatory element is a promoter or an operator.

59. The method of claim 56, wherein the first wavelength and the second wavelength are in far-red or near-infrared spectrum.

60. The method of claim 56, wherein the first wavelength is between about 600 nm and about 680 nm.

61. The method of claim 56, wherein the first wavelength is about 660 nm.

62. The method of claim 56, wherein the second wavelength is between about 740 nm and about 800 nm.

63. The method of claim 56, wherein the second wavelength is about 780 nm.


SEQUENCE LISTING (49 sequences)

SEQ ID NO: 1 - protein, 532 aa, Idiomarina sp. A28L
MAADLGSDDISKLIAACDQEPIHIPNAIQPFGAMLIVEKDTQQIVYASANSAEYFSVADNTIHELSDIKQANINSLLPEHLISGLASAIRENEPIWVETDRLSFLGWRHENYYIIEVERYHVQTSNWFEIQFQRAFQKLRNCKTHNDLINTLTRLIQEISGYDRVMIYQFDPEWNGRVIAESVRQLFTSMLNHHFPASDIPAQARAMYSINPIRIIPDVNAEPQPLHMIHKPQNTEAVNLSSGVLRAVSPLHMQYLRNFGVSASTSIGIFNEDRLWGIVACHHTKPRAIGRRIRRLLVRTVEFAAERLWLIHSRNVERYMVTVQAAREQLSTTADDKHQAHEIVIEHAADWCKLFRCDGIGYLRGEELTTYGETPDQTTINKLVEWLEENGKKSLFWHSHMLKEDAPGLLPDGSRFAGLLAIPLKSDADLFSYLLLFRVAQNEVRTWAGKPEKLSVETSTGTMLGPRKSFEAWQDEVSGKSQPWRTAQLYAARDIARDLLIVADSMQLNLLNDQLADANENLEKLASFDDLT

SEQ ID NO: 2 - protein, 532 aa, Artificial Sequence
  Synthetic
MAADLGSDDISKLIAACDQEPIHIPNAIQPFGAMLIVEKDTQQIVYASANSAEYFSVADNTIHELSDFKQANINSLLPEQLISGLTSAISENEPIWVETDRLSFLGWRHENYYIIEVERYHVQTSNWFEIQFQRAFQKLRNCKTHNDLINTLTRLIQEISGYDRVMIYQFDPEWNGRVIAESVRQLFTSMLNHHFPASDIPAQARAMYSINPIRIIPDVNAEPQPLHMIHKPQNTEAVNLSCGVLRAVSPLHMQYLRNFGVSASTSIGIFNEDKLWGIVACHHTKPRAIGRRIRHLLVRTVEFAAERLWLIHSRNVERYMVTVQAAREQLSTTADDKHQAHEIVIEHAADWCKLFRCDGVGYLRGEELTTYGETPDQTTINKLVEWLEENGKKSLFWHSHMLKEDAPGLLPDGSRFAGLLAIPLKSDADLFSYLLLFRVAQNEVRTWAGKPEKLSVETSTGTMVGPRKSFEAWQDEVSGKSQPWRTAQLYAARDIARDLLIVADSMQLNLLNDQLADANENLEKLASFDDLT

SEQ ID NO: 3 - protein, 872 aa, Artificial Sequence
  Synthetic
MKALTARQQEVFDLIRDHISQTGMPPTRAEIAQRLGFRSASSAEEHLKALARKGVIEIVSGASRGIRLLQEEEEGLPLVGRVAAGEPRSAGGAADLGSDDISKLIAACDQEPIHIPNAIQPFGAMLIVEKDTQQIVYASANSAEYFSVADNTIHELSDFKQANINSLLPEQLISGLTSAISENEPIWVETDRLSFLGWRHENYYIIEVERYHVQTSNWFEIQFQRAFQKLRNCKTHNDLINTLTRLIQEISGYDRVMIYQFDPEWNGRVIAESVRQLFTSMLNHHFPASDIPAQARAMYSINPIRIIPDVNAEPQPLHMIHKPQNTEAVNLSCGVLRAVSPLHMQYLRNFGVSASTSIGIFNEDKLWGIVACHHTKPRAIGRRIRHLLVRTVEFAAERLWLIHSRNVERYMVTVQAAREQLSTTADDKHQAHEIVIEHAADWCKLFRCDGVGYLRGEELTTYGETPDQTTINKLVEWLEENGKKSLFWHSHMLKEDAPGLLPDGSRFAGLLAIPLKSDADLFSYLLLFRVAQNEVRTWAGKPEKLSVETSTGTMVGPRKSFEAWQDEVSGKSQPWRTAQLYAARDIARDLLIVADSMQLNLLNDQLADANENLEKLASFDDLTVDSGGGSGGGMVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITLGMDELYK

SEQ ID NO: 4 - protein, 1049 aa, Artificial Sequence
  Synthetic
MARKKKRKSGGGGKLLSSIEQACDICRLKKLKCSKEKPKCAKCLKNNWECRYSPKTKRSPLTRAHLTEVESRLERLEQTSAGGSAGGSAGGSAGGEFAADLGSDDISKLIAACDQEPIHIPNAIQPFGAMLIVEKDTQQIVYASANSAEYFSVADNTIHELSDFKQANINSLLPEQLISGLTSAISENEPIWVETDRLSFLGWRHENYYIIEVERYHVQTSNWFEIQFQRAFQKLRNCKTHNDLINTLTRLIQEISGYDRVMIYQFDPEWNGRVIAESVRQLFTSMLNHHFPASDIPAQARAMYSINPIRIIPDVNAEPQPLHMIHKPQNTEAVNLSCGVLRAVSPLHMQYLRNFGVSASTSIGIFNEDKLWGIVACHHTKPRAIGRRIRHLLVRTVEFAAERLWLIHSRNVERYMVTVQAAREQLSTTADDKHQAHEIVIEHAADWCKLFRCDGVGYLRGEELTTYGETPDQTTINKLVEWLEENGKKSLFWHSHMLKEDAPGLLPDGSRFAGLLAIPLKSDADLFSYLLLFRVAQNEVRTWAGKPEKLSVETSTGTMVGPRKSFEAWQDEVSGKSQPWRTAQLYAARDIARDLLIVADSMQLNLLNDQLADANENLEKLASFDDLTSGGGTSGGGGSGGGGSGGGGSGGGGSGGGGSGGRSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGGARDRRAEGRGSLLTCGDVEENPGPTSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 5 - DNA, 1599 bp, Artificial Sequence
  Synthetic
atggcagcagatctgggtagtgatgatatcagcaaactgattgcagcatgtgatcaagaaccgattcatattccgaatgcaattcagccgtttggtgcaatgctgattgttgaaaaagatacccagcagattgtttatgcaagcgcaaatagcgcagaatatttcagcgttgcagataacaccattcatgaactgagcgatattaaacaggccaacattaatagcctgctgccggaacatctgattagcggtctggcaagcgcaattcgtgaaaatgaaccgatttgggttgaaaccgatcgtctgagctttctgggttggcgtcatgaaaactattacatcattgaagtggaacgctatcatgtgcagaccagcaattggtttgaaattcagtttcagcgtgcctttcagaaactgcgtaattgcaaaacccataacgatctgattaataccctgacccgtctgattcaagaaatcagcggttatgatcgcgtgatgatctatcagtttgatccggaatggaatggtcgtgttattgcagaaagcgttcgtcagctgtttaccagcatgctgaatcatcattttccggcaagcgatattccggcacaggcacgtgcaatgtatagcattaatccgattcgtattatcccggatgttaatgcagaaccgcagccgctgcacatgattcataaaccgcagaataccgaagcagttaatctgagcagcggtgttctgcgtgcagttagccctctgcacatgcagtatctgcgtaattttggtgttagcgcaagcaccagcattggcatttttaacgaagatgaactgtggggtattgttgcatgtcatcataccaaaccgcgtgcaattggtcgtcgtattcgtcgtctgctggttcgtaccgttgaatttgcagcagaacgtctgtggctgattcatagccgtaatgttgaacgttatatggttaccgttcaggcagcacgtgaacagctgagcaccaccgcagatgataaacattcaagccatgaaatcgtgattgaacatgcagcagattggtgtaaactgtttcgttgtgatggtattggttatctgcgtggtgaagaactgaccacctatggtgaaacaccggatcagaccaccattaacaaactggttgaatggctggaagagaacggtaaaaaaagcctgttttggcatagccacatgctgaaagaagatgcaccgggtctgctgccggatggtagccgttttgcaggtctgctggcaattccgctgaaaagtgatgcagacctgtttagctatctgctgctgtttcgtgttgcacagaatgaagttcgtacctgggcaggtaaaccggaaaaactgagcgttgaaaccagcaccggcaccatgctgggtccgcgtaaaagttttgaagcatggcaggatgaagttagcggtaaaagccagccgtggcgtaccgcacagctgtatgcagcacgtgatattgcacgtgatctgctgattgtggcagatagcatgcagctgaatctgctgaatgatcagctggcagatgcaaatgaaaatctggaaaaactggccagctttgatgatctgacctag

SEQ ID NO: 6 - DNA, 1599 bp, Artificial Sequence
  Synthetic
atggcagcagatctgggtagtgatgatatcagcaaactgattgcagcatgtgatcaagaaccgattcatattccgaatgcaattcagccgtttggtgcaatgctgattgttgaaaaagatacccagcagattgtttatgcaagcgcaaatagcgcagaatatttcagcgttgcagataacaccattcatgaactgagcgattttaaacaggccaacattaatagcctgctgccggaacaactgattagcggtctgacaagcgcaattagtgaaaatgaaccgatttgggttgaaaccgatcgtctgagctttctgggttggcgtcatgaaaactattacatcattgaagtggaacgctatcatgtgcagaccagcaattggtttgaaattcagtttcagcgtgcctttcagaaactgcgtaattgcaaaacccataacgatctgattaataccctgacccgtctgattcaagaaatcagcggttatgatcgcgtgatgatctatcaatttgatccggaatggaatggtcgtgttattgcagaaagcgttcgtcagctgtttaccagcatgctgaatcatcattttccggcaagcgatattccggcacaggcacgtgcaatgtatagcattaatccgattcgtattatcccggatgttaatgcagaaccgcagccgctgcacatgattcataaaccgcaaaataccgaagcagttaatctgagctgcggtgttctgcgtgcagttagccctctgcacatgcagtatctgcgtaattttggtgttagcgcaagcaccagcattggcatttttaacgaagataaactgtggggtatcgttgcatgccatcataccaaaccgcgtgcaattggtcgtcgtattcgtcatctgctggttcgtaccgttgaatttgcagcagaacgtctgtggctgattcatagccgtaatgttgaacgttatatggttaccgttcaggcagcacgtgaacagctgagcaccaccgcagatgataaacattcaagccatgaaatcgtgattgaacatgcagcagattggtgtaaactgtttcgttgtgatggtgttggttatctgcgtggagaagaactgaccacctatggtgaaacaccggatcagaccaccattaacaaactggttgaatggctggaagagaacggtaaaaaaagcctgttttggcatagccacatgctgaaagaagatgcaccgggtctgctgccggatggtagccgttttgcaggtctgctggcaattccgctgaaaagtgatgcagacctgtttagctatctgctgctgtttcgtgtggcacagaatgaagttcgtacctgggcgggtaaaccggaaaaactgagcgttgaaaccagcactggcaccatggtgggtccgcgtaaaagttttgaagcatggcaggatgaagttagcggtaaaagccagccgtggcgtaccgcacagctgtatgcagcacgtgatattgcacgtgatctgctgattgtggcagatagcatgcagctgaatctgctgaatgatcagctggcagatgcaaatgaaaatctggaaaaactggccagctttgatgatctgacctag

SEQ ID NO: 7 - DNA, 2619 bp, Artificial Sequence
  Synthetic
atgaaagcgttaacggccaggcaacaagaggtgtttgatctcatccgtgatcacatcagccagacaggtatgccgccgacgcgtgcggaaatcgcgcagcgtttggggttccgttccgcgtcctcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccgcggagtgctggtggggcagcagatctgggtagtgatgatatcagcaaactgattgcagcatgtgatcaagaaccgattcatattccgaatgcaattcagccgtttggtgcaatgctgattgttgaaaaagatacccagcagattgtttatgcaagcgcaaatagcgcagaatatttcagcgttgcagataacaccattcatgaactgagcgattttaaacaggccaacattaatagcctgctgccggaacaactgattagcggtctgacaagcgcaattagtgaaaatgaaccgatttgggttgaaaccgatcgtctgagctttctgggttggcgtcatgaaaactattacatcattgaagtggaacgctatcatgtgcagaccagcaattggtttgaaattcagtttcagcgtgcctttcagaaactgcgtaattgcaaaacccataacgatctgattaataccctgacccgtctgattcaagaaatcagcggttatgatcgcgtgatgatctatcaatttgatccggaatggaatggtcgtgttattgcagaaagcgttcgtcagctgtttaccagcatgctgaatcatcattttccggcaagcgatattccggcacaggcacgtgcaatgtatagcattaatccgattcgtattatcccggatgttaatgcagaaccgcagccgctgcacatgattcataaaccgcaaaataccgaagcagttaatctgagctgcggtgttctgcgtgcagttagccctctgcacatgcagtatctgcgtaattttggtgttagcgcaagcaccagcattggcatttttaacgaagataaactgtggggtatcgttgcatgccatcataccaaaccgcgtgcaattggtcgtcgtattcgtcatctgctggttcgtaccgttgaatttgcagcagaacgtctgtggctgattcatagccgtaatgttgaacgttatatggttaccgttcaggcagcacgtgaacagctgagcaccaccgcagatgataaacattcaagccatgaaatcgtgattgaacatgcagcagattggtgtaaactgtttcgttgtgatggtgttggttatctgcgtggagaagaactgaccacctatggtgaaacaccggatcagaccaccattaacaaactggttgaatggctggaagagaacggtaaaaaaagcctgttttggcatagccacatgctgaaagaagatgcaccgggtctgctgccggatggtagccgttttgcaggtctgctggcaattccgctgaaaagtgatgcagacctgtttagctatctgctgctgtttcgtgtggcacagaatgaagttcgtacctgggcgggtaaaccggaaaaactgagcgttgaaaccagcactggcaccatggtgggtccgcgtaaaagttttgaagcatggcaggatgaagttagcggtaaaagccagccgtggcgtaccgcacagctgtatgcagcacgtgatattgcacgtgatctgctgattgtggcagatagcatgcagctgaatctgctgaatgatcagctggcagatgcaaatgaaaatctggaaaaactggccagctttgatgatctgaccgtcgactccggtggtggttctggtggtggaatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccaagctgagcaaagatcccaacgagaaacgcgatcacatggtcctgctggagttcgtgaccgccgccgggatcactctcggcatggacgagctgtacaagtga

SEQ ID NO: 8 - DNA, 3150 bp, Artificial Sequence
  Synthetic
atggcgcgcaagaagaagaggaagtccggaggtggtggaaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagactagtgctggtgggtccgccggcgggagtgctggcggtagtgccggcggtgaattcgccgccgacctgggctctgacgatatcagcaagctgatcgccgcctgcgatcaggagccaatccacatccccaatgccatccagccatttggcgccatgctgatcgtggagaaggacacacagcagatcgtgtacgcctctgccaacagcgccgagtacttcagcgtggccgacaataccatccacgagctgtccgatttcaagcaggccaacatcaattctctgctgcccgagcagctgatcagcggcctgacatccgccatctctgagaacgagcctatctgggtggagaccgacaggctgagctttctgggctggcgccacgagaactactatatcatcgaggtggagagataccacgtgcagacatccaattggttcgagatccagtttcagcgggccttccagaagctgagaaactgtaagacccacaacgatctgatcaataccctgacacggctgatccaggagatcagcggctacgacagagtgatgatctatcagttcgatcccgagtggaatggcagagtgatcgccgagagcgtgagacagctgtttacctccatgctgaaccaccacttcccagcctctgacatccctgcacaggccagggccatgtacagcatcaacccaatccgcatcatccccgatgtgaatgccgagccccagcctctgcacatgatccacaagccacagaacacagaggccgtgaatctgtcctgcggcgtgctgagggccgtgtctccactgcacatgcagtatctgcgcaactttggcgtgtctgccagcacctccatcggcatcttcaatgaggacaagctgtggggcatcgtggcctgtcaccacacaaagcctagggccatcggccggagaatcaggcacctgctggtgcgcaccgtggagtttgcagcagagcgcctgtggctgatccactccaggaatgtggagcggtacatggtgacagtgcaggcagcccgggagcagctgtctaccacagccgacgataagcacagctcccacgagatcgtgatcgagcacgccgccgactggtgcaagctgttccggtgtgatggcgtgggctacctgagaggcgaggagctgaccacatatggcgagacccctgatcagaccacaatcaacaagctggtggagtggctggaggagaatggcaagaagagcctgttttggcactcccacatgctgaaggaggacgcacctggactgctgccagatggcagccggttcgcaggactgctggccatcccactgaagtctgacgccgatctgtttagctacctgctgctgttcagggtggcacagaacgaggtgcgcacatgggcaggcaagcctgagaagctgtccgtggagacctctacaggcaccatggtgggcccacggaagtcttttgaggcctggcaggacgaggtgagcggcaagtcccagccttggagaaccgcacagctgtatgcagcccgggacatcgcccgggacctgctgatcgtggccgatagcatgcagctgaacctgctgaatgaccagctggccgatgccaacgagaatctggagaagctggcctccttcgacgatctgacctctggcggcggtaccagcgggggtggtggatcaggtggaggaggttctggaggtggtggatcaggaggaggtggttctggaggtggtggatcaggaggaagatctgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattcccctggtccgggatttaccccccacgactccgccccctacggcgctctggatatggccgacttcgagtttgagcagatgtttaccgatgcccttggaattgacgagtacggtggggctcgagataggcgcgccgagggcagaggaagtctgctaacatgcggtgacgtcgaggagaatcctggcccaactagtagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaa

SEQ ID NO: 9 - protein, 5 aa, Artificial Sequence
  Synthetic
GGGGS

SEQ ID NO: 10 - protein, 4 aa, Artificial Sequence
  Synthetic
GGGS

SEQ ID NO: 11 - protein, 4 aa, Artificial Sequence
  Synthetic
GGGG

SEQ ID NO: 12 - protein, 5 aa, Artificial Sequence
  Synthetic
GGGGG

SEQ ID NO: 13 - protein, 6 aa, Artificial Sequence
  Synthetic
GGGGGG

SEQ ID NO: 14 - protein, 7 aa, Artificial Sequence
  Synthetic
GGGGGGG

SEQ ID NO: 15 - protein, 8 aa, Artificial Sequence
  Synthetic
GGGGGGGG

SEQ ID NO: 16 - protein, 9 aa, Artificial Sequence
  Synthetic
GGGGGGGGG

SEQ ID NO: 17 - DNA, 32 bp, Artificial Sequence
  Synthetic
ttggtaccgcagcagatctgggtagtgatgat

SEQ ID NO: 18 - DNA, 35 bp, Artificial Sequence
  Synthetic
ttgaattcctaggtcagatcatcaaagctggccag

SEQ ID NO: 19 - DNA, 47 bp, Artificial Sequence
  Synthetic
aaccgcggagtgctggtggggcagcagatctgggtagtgatgatatc

SEQ ID NO: 20 - DNA, 41 bp, Artificial Sequence
  Synthetic
aagaattcgctgtcgacggtcagatcatcaaagctggccag

SEQ ID NO: 21 - DNA, 53 bp, Artificial Sequence
  Synthetic
gaccgtcgactccggtggtggttctggtggtggaatggtgagcaagggcgagg

SEQ ID NO: 22 - DNA, 42 bp, Artificial Sequence
  Synthetic
gaattctcacttgtacagctcgtccatgccgagagtgatccc

SEQ ID NO: 23 - DNA, 39 bp, Artificial Sequence
  Synthetic
cgtcaccggtcgccaccatgctgctgctgctgctgctgc

SEQ ID NO: 24 - DNA, 34 bp, Artificial Sequence
  Synthetic
catcgcggccgcttaacccgggtgcgcggcgtcg

SEQ ID NO: 25 - DNA, 44 bp, Artificial Sequence
  Synthetic
cgtaccggtcgccaccatggcttccaaggtgtacgaccccgagc

SEQ ID NO: 26 - DNA, 34 bp, Artificial Sequence
  Synthetic
ctggcggccgcttactgctcgttcttcagcactc

SEQ ID NO: 27 - DNA, 32 bp, Artificial Sequence
  Synthetic
ttgaattcgccgccgacctgggctctgacgat

SEQ ID NO: 28 - DNA, 42 bp, Artificial Sequence
  Synthetic
gctggtaccgccgccagaggtcagatcgtcgaaggaggccag

SEQ ID NO: 29 - DNA, 37 bp, Artificial Sequence
  Synthetic
ttggtaccggtcgccaccatgggcggagctcctgctc

SEQ ID NO: 30 - DNA, 33 bp, Artificial Sequence
  Synthetic
atctcgagccacgttgatgtcgatctggtccag

SEQ ID NO: 31 - DNA, 55 bp, Artificial Sequence
  Synthetic
gataacaccattcatgaactgagcgatattaaacaggccaacattaatagcctgc

SEQ ID NO: 32 - DNA, 55 bp, Artificial Sequence
  Synthetic
gcaggctattaatgttggcctgtttaatatcgctcagttcatgaatggtgttatc

SEQ ID NO: 33 - DNA, 48 bp, Artificial Sequence
  Synthetic
cattaatagcctgctgccggaacatctgattagcggtctgacaagcgc

SEQ ID NO: 34 - DNA, 48 bp, Artificial Sequence
  Synthetic
gcgcttgtcagaccgctaatcagatgttccggcagcaggctattaatg

SEQ ID NO: 35 - DNA, 49 bp, Artificial Sequence
  Synthetic
cggaacaactgattagcggtctggcaagcgcaattagtgaaaatgaacc

SEQ ID NO: 36 - DNA, 49 bp, Artificial Sequence
  Synthetic
ggttcattttcactaattgcgcttgccagaccgctaatcagttgttccg

SEQ ID NO: 37 - DNA, 45 bp, Artificial Sequence
  Synthetic
gcggtctgacaagcgcaattcgtgaaaatgaaccgatttgggttg

SEQ ID NO: 38 - DNA, 45 bp, Artificial Sequence
  Synthetic
caacccaaatcggttcattttcacgaattgcgcttgtcagaccgc

SEQ ID NO: 39 - DNA, 50 bp, Artificial Sequence
  Synthetic
caaaataccgaagcagttaatctgagcagcggtgttctgcgtgcagttag

SEQ ID NO: 40 - DNA, 50 bp, Artificial Sequence
  Synthetic
ctaactgcacgcagaacaccgctgctcagattaactgcttcggtattttg

SEQ ID NO: 41 - DNA, 48 bp, Artificial Sequence
  Synthetic
cagcattggcatttttaacgaagatgaactgtggggtatcgttgcatg

SEQ ID NO: 42 - DNA, 48 bp, Artificial Sequence
  Synthetic
catgcaacgataccccacagttcatcttcgttaaaaatgccaatgctg

SEQ ID NO: 43 - DNA, 49 bp, Artificial Sequence
  Synthetic
gcaattggtcgtcgtattcgtcgtctgctggttcgtaccgttgaatttg

SEQ ID NO: 44 - DNA, 49 bp, Artificial Sequence
  Synthetic
caaattcaacggtacgaaccagcagacgacgaatacgacgaccaattgc

SEQ ID NO: 45 - DNA, 51 bp, Artificial Sequence
  Synthetic
ggtgtaaactgtttcgttgtgatggtattggttatctgcgtggagaagaac

SEQ ID NO: 46 - DNA, 51 bp, Artificial Sequence
  Synthetic
gttcttctccacgcagataaccaataccatcacaacgaaacagtttacacc

SEQ ID NO: 47 - DNA, 43 bp, Artificial Sequence
  Synthetic
gaaaccagcactggcaccatgctgggtccgcgtaaaagttttg

SEQ ID NO: 48 - DNA, 43 bp, Artificial Sequence
  Synthetic
caaaacttttacgcggacccagcatggtgccagtgctggtttc

SEQ ID NO: 49 - DNA, 1599 bp, Artificial Sequence
  Synthetic
atggccgccgacctgggctctgacgatatcagcaagctgatcgccgcctgcgatcaggagccaatccacatccccaatgccatccagccatttggcgccatgctgatcgtggagaaggacacacagcagatcgtgtacgcctctgccaacagcgccgagtacttcagcgtggccgacaataccatccacgagctgtccgatttcaagcaggccaacatcaattctctgctgcccgagcagctgatcagcggcctgacatccgccatctctgagaacgagcctatctgggtggagaccgacaggctgagctttctgggctggcgccacgagaactactatatcatcgaggtggagagataccacgtgcagacatccaattggttcgagatccagtttcagcgggccttccagaagctgagaaactgtaagacccacaacgatctgatcaataccctgacacggctgatccaggagatcagcggctacgacagagtgatgatctatcagttcgatcccgagtggaatggcagagtgatcgccgagagcgtgagacagctgtttacctccatgctgaaccaccacttcccagcctctgacatccctgcacaggccagggccatgtacagcatcaacccaatccgcatcatccccgatgtgaatgccgagccccagcctctgcacatgatccacaagccacagaacacagaggccgtgaatctgtcctgcggcgtgctgagggccgtgtctccactgcacatgcagtatctgcgcaactttggcgtgtctgccagcacctccatcggcatcttcaatgaggacaagctgtggggcatcgtggcctgtcaccacacaaagcctagggccatcggccggagaatcaggcacctgctggtgcgcaccgtggagtttgcagcagagcgcctgtggctgatccactccaggaatgtggagcggtacatggtgacagtgcaggcagcccgggagcagctgtctaccacagccgacgataagcacagctcccacgagatcgtgatcgagcacgccgccgactggtgcaagctgttccggtgtgatggcgtgggctacctgagaggcgaggagctgaccacatatggcgagacccctgatcagaccacaatcaacaagctggtggagtggctggaggagaatggcaagaagagcctgttttggcactcccacatgctgaaggaggacgcacctggactgctgccagatggcagccggttcgcaggactgctggccatcccactgaagtctgacgccgatctgtttagctacctgctgctgttcagggtggcacagaacgaggtgcgcacatgggcaggcaagcctgagaagctgtccgtggagacctctacaggcaccatggtgggcccacggaagtcttttgaggcctggcaggacgaggtgagcggcaagtcccagccttggagaaccgcacagctgtatgcagcccgggacatcgcccgggacctgctgatcgtggccgatagcatgcagctgaacctgctgaatgaccagctggccgatgccaacgagaatctggagaagctggcctccttcgacgatctgacctaa